(19) United States
(12) Patent Application Publication
(10) Pub. No.: US 2025/0064946 A1
(43) Pub. Date: Feb. 27, 2025

(54) PHARMACEUTICAL COMPOSITION COMPRISING A QUINAZOLINE COMPOUND

(71) Applicant: Astellas Pharma Inc., Tokyo (JP)

(72) Inventor: Takeyuki NAGASHIMA, Tokyo (JP)

(21) Appl. No.: 18/723,695

(22) PCT Filed: Dec. 23, 2022
(86) PCT No.: PCT/JP2022/048717

§ 371 Date: Jun. 24, 2024

(30) Foreign Application Priority Data
Dec. 24, 2021 (WO) PCT/JP2021/049036

(51) Int. Cl. A61K 47/55; A61P 35/00

(52) U.S. Cl. CPC A61K 47/55; A61P 35/00

(57) ABSTRACT

The present invention has an object to provide a pharmaceutical composition for treating a cancer of colorectal cancer and/or lung cancer.

The present inventors have studied about a compound that is useful as an active ingredient of a pharmaceutical composition for treating a cancer, and have found that a quinazoline compound has an excellent degradation-inducing action on a G12D mutant KRAS protein and a G12D mutant KRAS inhibition activity and that a pharmaceutical composition containing the compound as an active ingredient can be used as a therapeutic agent for a cancer, completing the present invention. The pharmaceutical composition of the present invention which contains a quinazoline compound or a salt thereof as an active ingredient can be used as a therapeutic agent for a cancer of colorectal cancer and/or lung cancer.

### TECHNICAL FIELD

The present invention relates to a pharmaceutical composition for treating colorectal cancer and/or lung cancer. The pharmaceutical composition according to the present invention contains a quinazoline compound which is excellent in a degradation-inducing action on a G12D mutant KRAS protein, and is useful as a G12D mutant KRAS inhibitor.

### BACKGROUND ART

Colorectal cancer is a cancer with a high morbidity and fatality in the world and about 1.4 million new cases are reported per year in the world (World Cancer Report 2014). The most effective means for treating colorectal cancer is a surgery, whereas chemotherapy, radiotherapy, and the like have recently been significantly advanced. Large scale clinical trials performed mainly in Europe and America have revealed that a combination chemotherapy in which several types of anticancer agents are combined is efficacious for colorectal cancer and contributes to regression of a tumor and prolongation of the prognosis (J. Clin. Oncol., 22, p. 229-237, 2004). In addition to the chemotherapy, a molecular target drug, such as an anti-VEGF (vascular endothelial growth factor) antibody or an anti-EGFR (epidermal growth factor receptor) antibody, is used as a first-line drug in combination with the chemotherapy. Regarding an anti-EGFR antibody drug, it has been apparent that mutation in a RAS gene is a negative predictive factor for the effect (Cancer Res., 66, p. 3992-3995, 2006), and in colorectal cancer, anti-EGFR antibody drugs are applicable only to patients with wild type RAS genes in the current situation.

In addition, the number of death due to lung cancer accounts for 19% of that due to all cancers which is the highest value, and about 1.8 million new patients are reported per year in the world (World Cancer Report 2014). In particular, patients of non-small cell lung cancer (NSCLC) are reported to account for 80 to 85% of those of lung cancer (American Cancer Society, Cancer Facts and Figures, 2016). Surgical therapy is considered until a certain stage, but surgery is rarely adopted after that stage and chemotherapy or radiotherapy then become a main therapy. Based on the cytomorphology, adenocarcinoma and squamous cell cancer are classified as the most typical type of NSCLC. These tumors follow a similar clinical course, but adenocarcinoma is characterized by localization in the lung periphery.

RAS proteins are low molecular weight guanosine triphosphate (GTP)-binding proteins of about 21 kDa constituted of 188-189 amino acids, and include four main types of proteins (KRAS (KRAS 4A and KRAS 4B), NRAS, and HRAS) produced by three genes of a KRAS gene, an NRAS gene, and an HRAS gene. RAS proteins exist in both an active GTP-binding type and an inactive GDP-binding type. A RAS protein is activated by replacement of guanosine diphosphate (GDP) with GTP due to, for example, ligand stimulation to a membrane receptor, such as EGFR. The active RAS binds to effector proteins as much as twenty, such as RAF, PI3K, and RALGDS, to activate the downstream signal cascade. On the other hand, the active RAS is converted to the inactive type by replacement of GTP with GDP due to the intrinsic GTP hydrolysis (GTPase) activity. The GTPase activity is enhanced by a GTPase-activating protein (GAP). As can be seen from the above statement, RAS bears an important function of “molecular switch” in an intracellular signal transduction pathway for EGFR or the like, and plays a critical role in the processes of cell growth, proliferation, angiogenesis, and the like (Nature Rev. Cancer, 2011, 11, p. 761-774, Nature Rev. Drug Discov., 2014, 13, p. 828-851, Nature Rev. Drug Discov., 2016, 15, p. 771-785).

Substitution of an amino acid by spontaneous mutation of the RAS gene results in a constant activated state due to hypofunction of RAS as GTPase or hyporeactivity to GAP, and then, signals are continuously sent downstream. The excessive signaling causes carcinogenesis or cancer growth acceleration. In 30 to 40% of the cases of colorectal cancer, a mutation is seen in a KRAS gene and in many cases, the mutation is a spontaneous point mutation particularly in the KRAS exon 2 (codon 12, codon 13) (Ann. Oncol., 27, p. 1746-1753, 2016). Effectiveness of existing anticancer agents has not been demonstrated on colorectal cancer with a KRAS mutation, and unmet medical needs for this segment are high. In addition, in lung cancer, a mutation of a RAS gene has been seen in 32% of the cases of lung adenocarcinoma. The breakdown of the frequency of the mutation is 96% in KRAS genes, 3% in NRAS genes, and 1% in HRAS genes, and it is reported that a majority of them are a spontaneous point mutation in the KRAS exon 2 (codon 12, codon 13) (Nature Rev. Drug Discov., 2014, 13, p. 828-851).

As a mutation of a KRAS gene, KRAS G12D mutation in which glycine at the codon 12 is substituted with aspartic acid and KRAS G12C mutation in which the glycine is substituted with cysteine are particularly known. In recent years, a number of G12C mutant-selective inhibitors have been developed, and among them, Sotorasib has been approved by FDA as a therapeutic agent for NSCLC (Drugs, 2021, 81, p. 1573-1579).

On the other hand, G12D mutant KRAS is seen in about 34% of the cases of pancreatic cancer, in 10% or more of the cases of colorectal cancer, and also in about 4% of the cases of lung adenocarcinoma (Nat. Rev. Cancer, 2018, 18, p. 767-777). Thus, a therapeutic agent for a KRAS mutation other than the KRAS G12C mutation is highly expected.

Patent Documents 1, 2, and 3 disclose KRAS inhibitors, and Patent Documents 2 and 3 disclose compounds represented by the following formulae (A) and (B), respectively (refer to the documents about the meanings of the signs in the formulae).

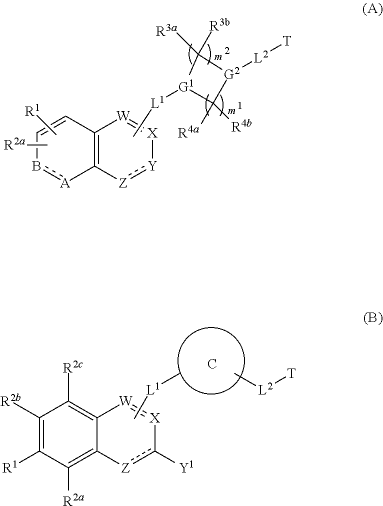

Patent Documents 1, 2, and 3 state that they are useful for a cancer with a mutation in the codon 12 of KRAS, and the G12D mutation is included as one of such mutations. However, the documents have no description about an action on a G12D mutant KRAS cancer.

In recent years, as a technique for inducing degradation of a target protein, bifunctional compounds collectively called as PROTAC (proteolysis-targeting chimera) or SNIPER (specific and nongenetic IAP-dependent protein eraser) are found and are expected as one novel technique of drug development modality (Drug. Discov. Today Technol., 2019, 31, p. 15-27). Such a bifunctional compound promotes formation of a composite of the target protein and an E3 ligase in a cell, and degradation of the target protein is induced by using the ubiquitin-proteasome system. The ubiquitin-proteasome system is one of intracellular protein degradation mechanisms. A protein called E3 ligase recognizes a protein to be degraded to convert the protein into ubiquitin, whereby degradation by proteasome is promoted.

Six hundreds or more E3 ligases are present in an organism, and are roughly divided into four types of HECT-domain E3s, U-box E3s, monomeric RING E3s, and multi-subunit E3s. E3 ligases used as a bifunctional degradation inducer which are called PROTAC, SNIPER, or the like are currently limited, and typical examples thereof include Von Hippel-Lindau (VHL), celebron (CRBN), inhibitor of apoptosis protein (IAP), and mouse double minute 2 homolog (MDM2). In particular, VHL is reported in Patent Document 4 and CRBN is reported in Patent Document 5.

The bifunctional compounds are compounds in which a ligand of a target protein and a ligand of an E3 ligase are bound via a linker, and some bifunctional compounds for degrading a KRAS protein have ever been reported (Non-patent Document 1, Non-patent Document 2, Patent Document 6, Patent Document 7, Patent Document 8, Patent Document 9).

### CITATION LIST

### Patent Document

[Patent Document 1] WO 2016/049565
[Patent Document 2] WO 2016/049568
[Patent Document 3] WO 2017/172979
[Patent Document 4] WO 2013/106643
[Patent Document 5] WO 2015/160845
[Patent Document 6] US Patent Application Publication No. 2018/0015087
[Patent Document 7] WO 2019/195609
[Patent Document 8] WO 2020/018788
[Patent Document 9] WO 2022/173032

### Non-Patent Document

[Non-patent Document 1] Cell. Chem. Biol., 2020, 27, p. 19-31
[Non-patent Document 2] ACS Cent. Sci., 2020, 6, p. 1367-1375

### SUMMARY OF INVENTION

### Technical Problem

An object of the present invention is to provide a pharmaceutical composition for treating colorectal cancer and/or lung cancer. In particular, an object of the present invention is to provide a technique for treating colorectal cancer and/or lung cancer by a compound that is excellent in a degradation-inducing action, for example, on a G12D mutant KRAS protein, and is useful as a G12D mutant KRAS inhibitor.

### Solution to Problem

As a result of intensive and extensive studies about a compound useful as an active ingredient of a pharmaceutical composition for treating a cancer for the purpose of providing a pharmaceutical composition for treating a cancer of colorectal cancer and/or lung cancer, in particular, a pharmaceutical composition for treating a G12D mutant KRAS-positive cancer, the present inventors have found that the quinazoline compound of the formula (I) has an excellent degradation-inducing action on a G12D mutant KRAS protein and a G12D mutant KRAS inhibition activity and that a pharmaceutical composition containing the compound as an active ingredient is useful as a pharmaceutical composition for treating a cancer of colorectal cancer and/or lung cancer. Furthermore, the present inventors have found that a bifunctional compound of the formula (I) characterized in that a substituent on the position 8 of quinazoline is bound to a ligand of an E3 ligase or in that a substituent on the position 8 of quinazoline is bound to a ligand of an E3 ligase via a linker has an excellent degradation-inducing action on a G12D mutant KRAS protein and a G12D mutant KRAS inhibition activity, and that a pharmaceutical composition containing the compound as an active ingredient is useful as a pharmaceutical composition for treating a cancer of colorectal cancer and/or lung cancer, thus completing the present invention.

Specifically, the present invention relates to a pharmaceutical composition for treating colorectal cancer and/or lung cancer, the composition comprising a compound of the following formula (I) or a salt thereof and one or more pharmaceutically acceptable excipients.

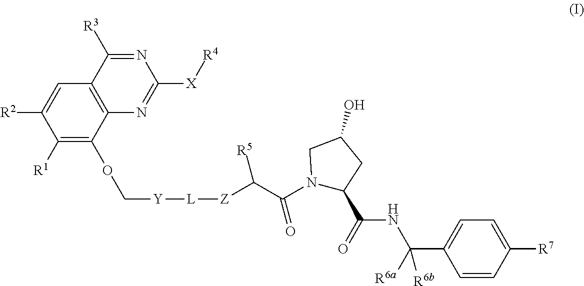

In the formula (I), R¹ is the following formula (II) or (III):

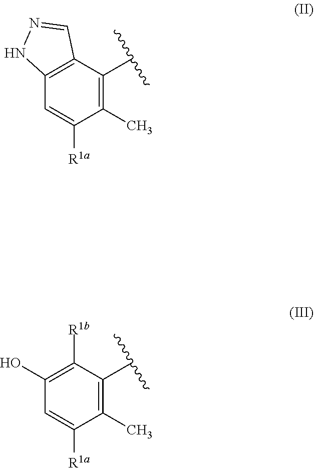

R^{1a} and R^{1b}, which are the same as or different from each other, are H or F,
R² is halogen, C₁₋₃ alkyl, cyclopropyl, or vinyl,
R³ is the following formula (IV):

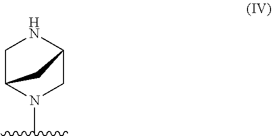 [SMILES: *N1C[C@@H]2C[C@H]1CN2]

R⁴ is C₁₋₃ alkyl optionally substituted with OCH₃, oxetanyl, tetrahydrofuranyl, tetrahydropyranyl, optionally substituted pyrazolyl, optionally substituted pyridyl, optionally substituted pyrimidinyl, optionally substituted pyrrolidinyl, or optionally substituted piperidinyl,
R⁵ is ethyl, isopropyl, tert-butyl, or C₃₋₆ cycloalkyl,
R^{6a} and R^{6b}, which are the same as or different from each other, are H or C₁₋₃ alkyl optionally substituted with a group selected from the group consisting of F, OH, and N(CH₃)₂, or
R^{6a} and R^{6b} form cyclopropyl together with the carbon to which they are attached,
R⁷ is H, halogen, or a group selected from the group consisting of the following formulae (VI), (VII), (VIII), and (IX):

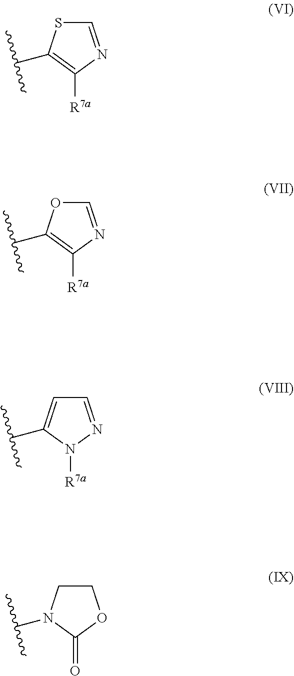

R^{7a} is H or C₁₋₃ alkyl optionally substituted with OH,
X is O,
Y is phenylene or pyridinediyl,
L is a bond, C₁₋₃ alkylene, or C=O,
Z is NH or a group selected from the group consisting of the following formulae (X), (XI), and (XII):

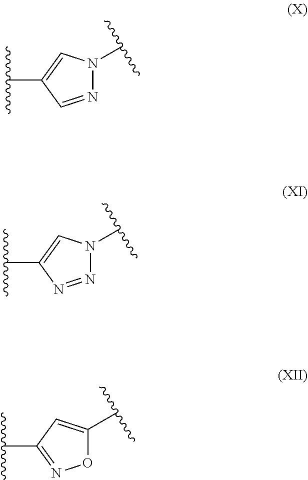 [SMILES: *c1cnn(*)c1 ; *c1cn(*)nn1 ; *c1cc(*)on1]

or Y-L-Z integrally forms the following formula (XIII):

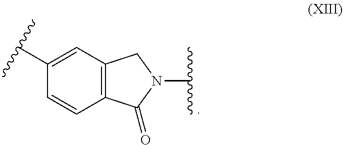 [SMILES: *c1ccc2c(c1)CN(*)C2=O]

Furthermore, the present invention relates to a pharmaceutical composition for treating colorectal cancer and/or lung cancer, the composition comprising a compound of the following formula (I) or a salt thereof.

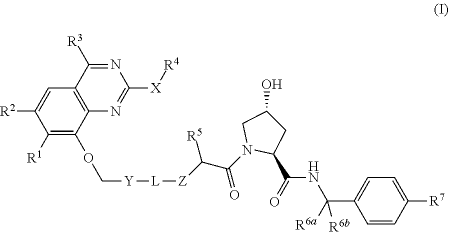

In the formula (I), R¹ is the following formula (II) or (III):

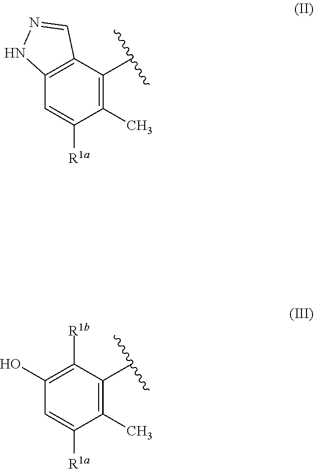

R^{1a} and R^{1b}, which are the same as or different from each other, are H or F,
R² is halogen, C₁₋₃ alkyl, cyclopropyl, or vinyl,
R³ is the following formula (IV):

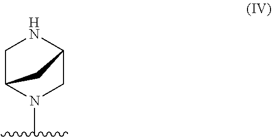 [SMILES: *N1C[C@@H]2C[C@H]1CN2]

R⁴ is C₁₋₃ alkyl, oxetanyl, tetrahydrofuranyl, tetrahydropyranyl, optionally substituted pyrazolyl, optionally substituted pyridyl, optionally substituted pyrimidinyl, optionally substituted pyrrolidinyl, or optionally substituted piperidinyl,
R⁵ is ethyl, isopropyl, tert-butyl, or C₃₋₆ cycloalkyl,
R^{6a} and R^{6b}, which are the same as or different from each other, are H or C₁₋₃ alkyl optionally substituted with a group selected from the group consisting of F, OH, and N(CH₃)₂, or R^{6a} and R^{6b} form cyclopropyl together with the carbon to which they are attached,
R⁷ is H, halogen, or a group selected from the group consisting of the following formulae (VI), (VII), (VIII), and (IX):

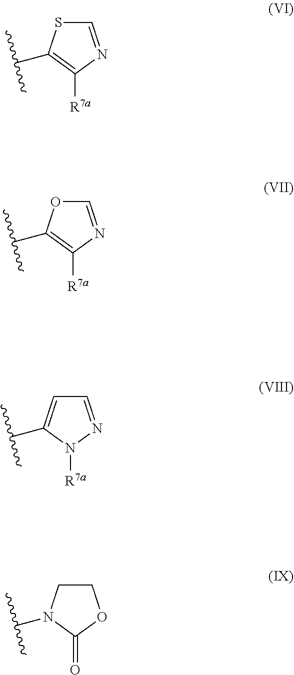

R^{7a} is H or C₁₋₃ alkyl optionally substituted with OH,
X is O,
Y is phenylene or pyridinediyl,
L is a bond, C₁₋₃ alkylene, or C=O,
Z is NH or a group selected from the group consisting of the following formulae (X), (XI), and (XII);

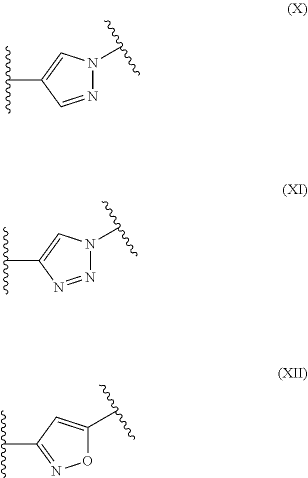 [SMILES: *c1cnn(*)c1 ; *c1cn(*)nn1 ; *c1cc(*)on1]

or Y-L-Z integrally forms the following formula (XIII):

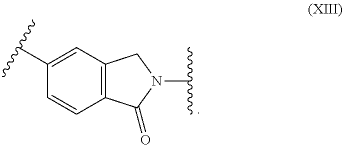 [SMILES: *c1ccc2c(c1)CN(*)C2=O]

Note that, when a sign in a chemical formula herein is used in another chemical formula, the same sign represents the same meaning unless otherwise specified.

The present invention also relates to a pharmaceutical composition, in particular, a pharmaceutical composition for treating a cancer of colorectal cancer and/or lung cancer, in particular, a pharmaceutical composition for treating a G12D mutant KRAS-positive cancer, the pharmaceutical composition comprising the compound of the formula (I) or a salt thereof and one or more pharmaceutically acceptable excipients. Note that the pharmaceutical composition includes a therapeutic agent for a cancer of colorectal cancer and/or lung cancer, in particular, a G12D mutant KRAS-positive cancer, the therapeutic agent comprising the compound of the formula (I) or a salt thereof.

In addition, the present invention also relates to use of the compound of the formula (I) or a salt thereof for the manufacture of a pharmaceutical composition for treating a Cancer of colorectal cancer and/or lung cancer, in particular, a G12D mutant KRAS-positive cancer, use of the compound of the formula (I) or a salt thereof for treating a cancer of colorectal cancer and/or lung cancer, in particular, a G12D mutant KRAS-positive cancer, the compound of the formula (I) or a salt thereof for use in the treatment of a cancer of colorectal cancer and/or lung cancer, in particular, a G12D mutant KRAS-positive cancer, and, a method for treating a cancer of colorectal cancer and/or lung cancer, in particular, a G12D mutant KRAS-positive cancer, the method including administering an effective amount of the compound of the formula (I) or a salt thereof to a subject.

The present invention also relates to a pharmaceutical composition comprising the compound of the formula (I) or a salt thereof that is a G12D mutant KRAS protein degradation inducer and/or a G12D mutant KRAS inhibitor, the pharmaceutical composition comprising the compound of the formula (I) or a salt thereof for use as a G12D mutant KRAS protein degradation inducer and/or a G12D mutant KRAS inhibitor, and the pharmaceutical composition comprising a G12D mutant KRAS protein degradation inducer and/or a G12D mutant KRAS inhibitor containing the compound of the formula (I) or a salt thereof.

Note that the “subject” is a human or another animal that needs the treatment, and in an embodiment, the “subject” is a human who needs the prevention or treatment.

The present invention also relates to use of the compound of the formula (I) or a salt thereof for the manufacture of a pharmaceutical composition for treating colorectal cancer and/or lung cancer, use of the compound of the formula (I) or a salt thereof for treating colorectal cancer and/or lung cancer, the compound of the formula (I) or a salt thereof for use in the treatment of colorectal cancer and/or lung cancer, the compound of the formula (I) or a salt thereof for treating colorectal cancer and/or lung cancer, and a method for treating colorectal cancer and/or lung cancer, the method including administering an effective amount of the compound of the formula (I) or a salt thereof to a subject.

### Advantageous Effects of Invention

The compound of the formula (I) or a salt thereof has a degradation-inducing action on a G12D mutant KRAS protein and a G12D mutant KRAS inhibition activity, and can be used as an active ingredient of a pharmaceutical composition for treating a cancer of colorectal cancer and/or lung cancer, in particular, a G12D mutant KRAS-positive cancer.

### DESCRIPTION OF EMBODIMENTS

The present invention will be described in detail below.

As used herein, “optionally substituted” means being unsubstituted or having one to three substituents. Note that when there are multiple substituents, the substituents may be the same as or different from each other.

“C₁₋₃ Alkyl” is linear or branched alkyl having 1 to 3 carbon atoms, and examples thereof include methyl, ethyl, n-propyl, and isopropyl. In an embodiment, “C₁₋₃ Alkyl” is methyl, ethyl, or isopropyl, in an embodiment, “C₁₋₃ Alkyl” is methyl or ethyl, in an embodiment, “C₁₋₃ Alkyl” is methyl or isopropyl, in an embodiment, “C₁₋₃ Alkyl” is ethyl or isopropyl, in an embodiment, “C₁₋₃ Alkyl” is ethyl or n-propyl, in an embodiment, “C₁₋₃ Alkyl” is methyl, in an embodiment, “C₁₋₃ Alkyl” is ethyl, in an embodiment, “C₁₋₃ Alkyl” is n-propyl, and in an embodiment, “C₁₋₃ Alkyl” is isopropyl.

“C₃₋₆ Cycloalkyl” is cycloalkyl having 3 to 6 carbon atoms, and examples thereof include cyclopropyl, cyclobutyl, cyclopentyl, and cyclohexyl. In an embodiment, “C₃₋₆ Cycloalkyl” is cyclobutyl, cyclopentyl, or cyclohexyl, in an embodiment, “C₃₋₆ Cycloalkyl” is cyclobutyl or cyclopentyl, in an embodiment, “C₃₋₆ Cycloalkyl” is cyclopentyl or cyclohexyl, in an embodiment, “C₃₋₆ Cycloalkyl” is cyclopropyl, in an embodiment, “C₃₋₆ Cycloalkyl” is cyclobutyl, in an embodiment, “C₃₋₆ Cycloalkyl” is cyclopentyl, and in an embodiment, “C₃₋₆ Cycloalkyl” is cyclohexyl.

“C₁₋₃ Alkylene” is linear or branched C₁₋₃ alkylene, and examples thereof include methylene, ethylene, trimethylene, propylene, methylmethylene, and 1,1-dimethylmethylene. In an embodiment, “C₁₋₃ Alkylene” is linear or branched C₁₋₃ alkylene, and in an embodiment, “C₁₋₃ Alkylene” is methylene, ethylene, trimethylene, or propylene. In addition, “C₁₋₃ alkylene” is linear or branched C₁₋₃ alkylene, and in an embodiment, “C₁₋₃ Alkylene” is methylene or ethylene, in an embodiment, “C₁₋₃ Alkylene” is methylene, and in an embodiment, “C₁₋₃ Alkylene” is ethylene.

“Halogen” means F, Cl, Br, and I. In an embodiment, “Halogen” is F, Cl, or Br, in an embodiment, “Halogen” is F, in an embodiment, “Halogen” is Cl, and in an embodiment, “Halogen” is Br.

In an embodiment, a substituent acceptable in “optionally substituted pyrazolyl”, “optionally substituted pyridyl”, and “optionally substituted pyrimidinyl” is C₁₋₃ alkyl. In an embodiment, the substituent is methyl or ethyl, in an embodiment, the substituent is methyl, and in an embodiment, the substituent is ethyl.

In an embodiment, a substituent acceptable in “optionally substituted pyrrolidinyl” and “optionally substituted piperidinyl” is C₁₋₃ alkyl optionally substituted with F or oxetanyl. In an embodiment, the substituent is C₁₋₃ alkyl optionally substituted with F, in an embodiment, the substituent is methyl, ethyl, difluoroethyl, or oxetanyl, in an embodiment, the substituent is difluoroethyl or oxetanyl, in an embodiment, the substituent is 2,2-difluoroethyl, and in an embodiment, the substituent is oxetanyl.

In an embodiment, “C₁₋₃ alkyl optionally substituted with F” is methyl optionally substituted with F or ethyl optionally substituted with F. Examples thereof include monofluoromethyl, difluoromethyl, trifluoromethyl, monofluoroethyl, difluoroethyl, and trifluoroethyl. In an embodiment, “C₁₋₃ alkyl optionally substituted with F” is methyl, ethyl, monofluoromethyl, difluoromethyl, or difluoroethyl, in an embodiment, “C₁₋₃ alkyl optionally substituted with F” is monofluoromethyl or difluoromethyl, in an embodiment, “C₁₋₃ alkyl optionally substituted with F” is monofluoromethyl or difluoroethyl, in an embodiment, “C₁₋₃ alkyl optionally substituted with F” is difluoromethyl or difluoroethyl, in an embodiment, “C₁₋₃ alkyl optionally substituted with F” is monofluoromethyl, in an embodiment, “C₁₋₃ alkyl optionally substituted with F” is difluoromethyl, in an embodiment, “C₁₋₃ alkyl optionally substituted with F” is difluoroethyl, and in an embodiment, “C₁₋₃ alkyl optionally substituted with F” is 2,2-difluoroethyl.

In an embodiment, “C₁₋₃ alkyl optionally substituted with OH” is methyl optionally substituted with one OH group or ethyl optionally substituted with one to two OH groups. Examples thereof include hydroxymethyl, 1-hydroxyethyl, 2-hydroxyethyl, and 1,2-hydroxyethyl. In an embodiment, “C₁₋₃ alkyl optionally substituted with OH” is hydroxymethyl or hydroxyethyl, in an embodiment, “C₁₋₃ alkyl optionally substituted with OH” is hydroxymethyl, and in an embodiment, “C₂₋₃ alkyl optionally substituted with OH” is hydroxyethyl.

In an embodiment, “C₁₋₃ Alkyl optionally substituted with OCH₃” is methyl optionally substituted with one OCH₃ group or ethyl optionally substituted with one or two OCH₃ groups or n-propyl optionally substituted with one or two OCH₃ groups. Examples thereof include methyl, ethyl, n-propyl, isopropyl, methoxymethyl, 1-methoxyethyl, 2-methoxyethyl, 1,2-dimethoxyethyl, 1-methoxypropyl, 2-methoxypropyl, 3-methoxypropyl, 1,2-dimethoxypropyl and 1,3-dimethoxypropyl. In an embodiment, “C₁₋₃ alkyl optionally substituted with OCH₃” is ethyl or 2-methoxypropyl, and in an embodiment “C₁₋₃ alkyl optionally substituted with OCH₃” is 2-methoxypropyl.

In an embodiment, “C₁₋₃ alkyl optionally substituted with N(CH₃)₂” is methyl optionally substituted with one N(CH₃)₂ group or ethyl optionally substituted with one N(CH₃)₂ group. In an embodiment, “C₁₋₃ alkyl optionally substituted with N(CH₃)₂” is dimethylaminomethyl, and in an embodiment, “C₁₋₃ alkyl optionally substituted with N(CH₃)₂” is dimethylaminoethyl.

“G12D Mutation” represents a mutation in which the amino acid residue corresponding to the codon 12 in a wild type protein is converted from glycine to aspartic acid.

“G12D Mutant KRAS” represents KRAS having the “G12D mutation”.

“Colorectal cancer” is a malignant tumor occurring in the large intestine, and “lung cancer” is a malignant tumor occurring in the lung. “Pancreatic cancer” is a malignant tumor occurring in the pancreas. Examples thereof include pancreatic ductal carcinoma and pancreatic ductal adenocarcinoma. In an embodiment, “Pancreatic cancer” is pancreatic ductal carcinoma, and in an embodiment, “Pancreatic Cancer” is pancreatic ductal adenocarcinoma.

In an embodiment, colorectal cancer and lung cancer is a metastatic, locally advanced, recurrent, and/or refractory cancer. In an embodiment, colorectal cancer and Lung cancer is a cancer of a patient who has been untreated or who has a medical history.

In an embodiment, colorectal cancer is colon cancer or rectal cancer. In an embodiment, lung cancer is small cell lung cancer or non-small cell lung cancer.

“G12D mutant KRAS-positive cancer” is a G12D mutant KRAS-positive cancer, and, for example, is a cancer in which KRAS G12D mutation occurs and a cancer which has a high positive rate for G12D mutant KRAS. In an embodiment, “G12D mutant KRAS-positive cancer” is G12D mutant KRAS-positive colorectal cancer and/or lung cancer.

Embodiments of the compound of the formula (I) or a salt thereof contained in the pharmaceutical composition of the present invention will be shown below.
(1) The compound or a salt thereof in which R¹ is the formulae (II) or (III) and R^{1a} and R^{1b}, which are the same as or different from each other, are H or F.

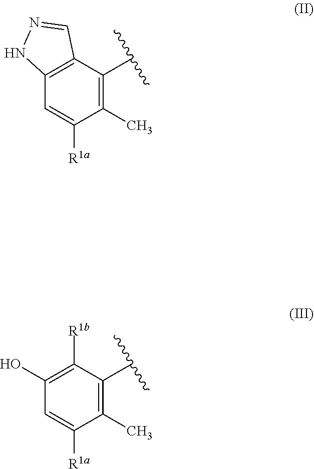

In an embodiment, the compound or a salt thereof in which R¹ is the formula (II) and R^{1a} is H or F. In an embodiment, the compound or a salt thereof in which R¹ is the formula (II) and R^{1a} is H. In an embodiment, the compound or a salt thereof in which R¹ is the formula (II) and R^{1a} is F. In an embodiment, the compound or a salt thereof in which R¹ is the formula (III) and R^{1a} and R^{1b}, which are the same as or different from each other, are H or F. In an embodiment, the compound or a salt thereof in which R¹ is the formula (III) and R^{1a} and R^{1b} are each H. In an embodiment, the compound or a salt thereof in which R¹ is the formula (III), R^{1a} is H, and R^{1b} is F. In an embodiment, the compound or a salt thereof in which R¹ is the formula (III) and R^{1a} and R^{1b} are each F. In an embodiment, the compound or a salt thereof in which R¹ is the formula (III), R^{1a} is F, and R^{1b} is H.
(2) The compound or a salt thereof in which R² is halogen, C₁₋₃ alkyl, cyclopropyl, or vinyl. In an embodiment, the compound or a salt thereof in which R² is halogen. In an embodiment, the compound or a salt thereof in which R² is C₁₋₃ alkyl. In an embodiment, the compound or a salt thereof in which R² is cyclopropyl. In an embodiment, the compound or a salt thereof in which R² is vinyl.
(3) The compound or a salt thereof in which R³ is the formula (IV).

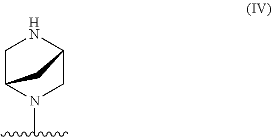 [SMILES: *N1C[C@@H]2C[C@H]1CN2]

(4) The compound or a salt thereof in which R⁴ is C₁₋₃ alkyl optionally substituted with OCH₃, oxetanyl, tetrahydrofuranyl, tetrahydropyranyl, optionally substituted pyrazolyl, optionally substituted pyridyl, optionally substituted pyrimidinyl, optionally substituted pyrrolidinyl, or optionally substituted piperidinyl. In an embodiment, the compound or a salt thereof in which R⁴ is C₁₋₃ alkyl, oxetanyl, tetrahydrofuranyl, tetrahydropyranyl, optionally substituted pyrazolyl, optionally substituted pyridyl, optionally substituted pyrimidinyl, optionally substituted pyrrolidinyl, or optionally substituted piperidinyl. In an embodiment, the compound or a salt thereof in which R⁴ is tetrahydrofuranyl, tetrahydropyranyl, optionally substituted pyrrolidinyl, or optionally substituted piperidinyl. In an embodiment, the compound or a salt thereof in which R⁴ is tetrahydrofuranyl, tetrahydropyranyl, or optionally substituted piperidinyl. In an embodiment, the compound or a salt thereof in which R⁴ is tetrahydropyranyl or optionally substituted piperidinyl. In an embodiment, the compound or a salt thereof in which R⁴ is C₁₋₃ alkyl optionally substituted with OCH₃ or tetrahydropyranyl. In an embodiment, the compound or a salt thereof in which R⁴ is tetrahydropyranyl or 2-methoxypropyl. In an embodiment, the compound or a salt thereof in which R⁴ is C₁₋₃ alkyl optionally substituted with OCH₃. In an embodiment, the compound or a salt thereof in which R⁴ is C₁₋₃ alkyl. In an embodiment, the compound or a salt thereof in which R⁴ is oxetanyl. In an embodiment, the compound or a salt thereof in which R⁴ is tetrahydrofuranyl. In an embodiment, the compound or a salt thereof in which R⁴ is tetrahydropyranyl. In an embodiment, the compound or a salt thereof in which R⁴ is optionally substituted pyrazolyl. In an embodiment, the compound or a salt thereof in which R⁴ is optionally substituted pyridyl. In an embodiment, the compound or a salt thereof in which R⁴ is optionally substituted pyrimidinyl. The compound or a salt thereof in which R⁴ is optionally substituted pyrrolidinyl. In an embodiment, the compound or a salt thereof in which R⁴ is optionally substituted piperidinyl. In an embodiment, the compound or a salt thereof in which R⁴ is 2-methoxypropyl.
(5) The compound or a salt thereof in which R⁵ is ethyl, isopropyl, tert-butyl, or C₃₋₆ cycloalkyl. In an embodiment, the compound or a salt thereof in which R⁵ is isopropyl, tert-butyl, or C₃₋₆ cycloalkyl. In an embodiment, the compound or a salt thereof in which R⁵ is isopropyl or tert-butyl. In an embodiment, the compound or a salt thereof in which R⁵ is isopropyl. In an embodiment, the compound or a salt thereof in which R⁵ is tert-butyl. In an embodiment, the compound or a salt thereof in which R⁵ is C₃₋₆ cycloalkyl.
(6) The compound or a salt thereof in which R^{6a} and R^{6b}, which are the same as or different from each other, are H or C₁₋₃ alkyl optionally substituted with a group selected from the group consisting of F, OH, and N(CH₃)₂, or R^{6a} and R^{6b} form a cyclopropyl together with the carbon to which they are attached. In an embodiment, the compound or a salt thereof in which R^{6a} and R^{6b}, which are the same as or different from each other, are H or C₁₋₃ alkyl optionally substituted with a group selected from the group consisting of F, OH, and N(CH₃)₂. In an embodiment, the compound or a salt thereof in which R^{6a} and R^{6b}, which are the same as or different from each other, are C₁₋₃ alkyl optionally substituted with a group selected from the group consisting of F, OH, and N(CH₃)₂. In an embodiment, the compound or a salt thereof in which R^{6a} and R^{6b} are each H. In an embodiment, the compound or a salt thereof in which R^{6a} is H and R^{6b} is C₁₋₃ alkyl optionally substituted with a group selected from the group consisting of F, OH, and N(CH₃)₂. In an embodiment, the compound or a salt thereof in which R^{6a} is H and R^{6b} is C₁₋₃ alkyl optionally substituted with F. In an embodiment, the compound or a salt thereof in which R^{6a} is H and RED is C₁₋₃ alkyl optionally substituted with OH. In an embodiment, the compound or a salt thereof in which R^{6a} is H and R^{6b} is C₁₋₃ alkyl optionally substituted with N(CH₃)₂. In an embodiment, the compound or a salt thereof in which R^{6a} and R^{6b} form a cyclopropyl together with the carbon to which they are attached.
(7) The compound or a salt thereof in which R⁷ is H, halogen, or a group selected from the group consisting of the formula (VI), the formula (VII), the formula (VIII), and the formula (IX).

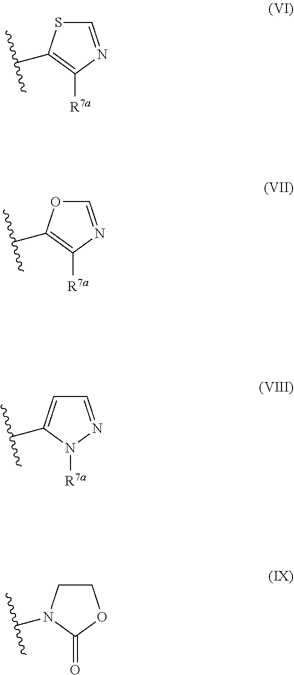

In an embodiment, the compound or a salt thereof in which R⁷ is H. In an embodiment, the compound or a salt thereof in which R⁷ is halogen. In an embodiment, the compound or a salt thereof in which R⁷ is a group selected from the group consisting of the formula (VI), the formula (VII), the formula (VIII), and the formula (IX). In an embodiment, the compound or a salt thereof in which R⁷ is the formula (VI), (VIII), or (IX). In an embodiment, the compound or a salt thereof in which R⁷ is the formula (VI) or (VIII). In an embodiment, the compound or a salt thereof in which R⁷ is the formula (VI) or (IX). In an embodiment, the compound or a salt thereof in which R⁷ is the formula (VI). In an embodiment, the compound or a salt thereof in which R⁷ is the formula (VII). In an embodiment, the compound or a salt thereof in which R⁷ is the formula (VIII). In an embodiment, the compound or a salt thereof in which R⁷ is the formula (IX).
(8) The compound or a salt thereof in which R^{1a} is H or C₁₋₃ alkyl optionally substituted with OH. In an embodiment, the Compound or a salt thereof in which R^{7a} is H. In an embodiment, the compound or a salt thereof in which R^{7a} is C₁₋₃ alkyl optionally substituted with OH. In an embodiment, the compound or a salt thereof in which R^{1a} is C₁₋₃ alkyl.
(9) The compound or a salt thereof in which X is O.
(10) The compound or a salt thereof in which Y is phenylene or pyridinediyl. In an embodiment, the compound or a salt thereof in which Y is 1,4-phenylene or 2,5-pyridinediyl. In an embodiment, the compound or a salt thereof in which Y is phenylene. In an embodiment, the compound or a salt thereof in which Y is 1,4-phenylene. In an embodiment, the compound or a salt thereof in which Y is pyridinediyl. In an embodiment, the compound or a salt thereof in which Y is 2,5-pyridinediyl.
(11) The compound or a salt thereof in which L is a bond, C₁₋₃ alkylene, or C═O. In an embodiment, the compound or a salt thereof in which L is a bond or C₁₋₃ alkylene. In an embodiment, the compound or a salt thereof in which L is a bond or C═O. In an embodiment, the compound or a salt thereof in which L is a bond. In an embodiment, the compound or a salt thereof in which L is C₁₋₃ alkylene. In an embodiment, the compound or a salt thereof in which L is C═O.
(12) The compound or a salt thereof in which z is NH or a group selected from the group consisting of the formula (X), the formula (XI), and the formula (XII).

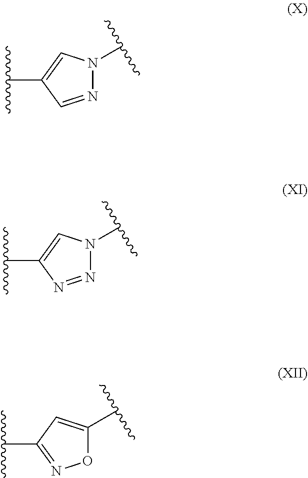 [SMILES: *c1cnn(*)c1 ; *c1cn(*)nn1 ; *c1cc(*)on1]

In an embodiment, the compound or a salt thereof in which z is NH or a group selected from the group consisting of the formula (X)-1, the formula (XI)-1, and the formula (XII)-1.

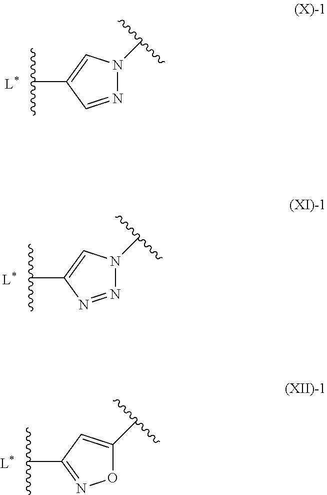 [SMILES: *c1cnn(*)c1 ; *c1cn(*)nn1 ; *c1cc(*)on1]

(In the formulae, L* represents linking to L.)

In an embodiment, the compound or a salt thereof in which Z is a group selected from the group consisting of the formula (X), the formula (XI), and the formula (XII). In an embodiment, the compound or a salt thereof in which Z is a group selected from the group consisting of the formula (X)-1, the formula (XI)-1, and the formula (XII)-1. In an embodiment, the compound or a salt thereof in which Z is the formula (X) or the formula (XI). In an embodiment, the compound or a salt thereof in which Z is the formula (X)-1 or the formula (XI)-1. In an embodiment, the compound or a salt thereof in which Z is the formula (XI) or the formula (XII). In an embodiment, the compound or a salt thereof in which Z is the formula (XI)-1 or the formula (XII)-1. In an embodiment, the compound or a salt thereof in which Z is NH. In an embodiment, the compound or a salt thereof in which Z is the formula (X). In an embodiment, the compound or a salt thereof in which Z is the formula (X)-1. In an embodiment, the compound or a salt thereof in which Z is the formula (XI). In an embodiment, the compound or a salt thereof in which Z is the formula (XI)-1. In an embodiment, the compound or a salt thereof in which Z is the formula (XII). In an embodiment, the compound or a salt thereof in which Z is the formula (XII)-1.
(13) The compound or a salt thereof in which Y-L-Z is the following formula (XIII).

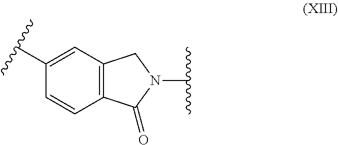 [SMILES: *c1ccc2c(c1)CN(*)C2=O]

In an embodiment, the compound or a salt thereof in which Y-L-Z is the following formula (XIII)-1.

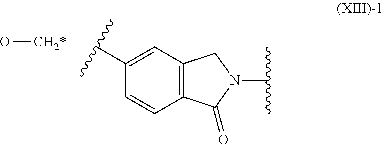

(In the formula, O—CH₂* represents linking to the carbon in O—CH₂.)
(14) The compound or a salt thereof which is a combination of any compatible two or more of the aspects of the above (1) to (13).

Specific examples of compounds included in the present invention in an embodiment, include the following compounds.
(15) The compound or a salt thereof in which R¹ is the formula (II), R^{1a} is F, R² is cyclopropyl, R³ is the formula (IV), R⁴ is tetrahydropyranyl or optionally substituted piperidinyl, R⁵ is isopropyl, R^{6a} is H, R^{6b} is C₁₋₃ alkyl optionally substituted with OH, R⁷ is the formula (VI), (VIII), or (IX), R^{7a} is C₁₋₃ alkyl, Y is phenylene, L is a bond, and 2 is the formula (XI).
(16) The compound or a salt thereof in which R¹ is the formula (II), R^{1a} is F, R² is cyclopropyl, R³ is the formula (IV), R⁴ is tetrahydropyranyl, R⁵ is isopropyl, R^{6a} is H, R^{6b} is C₁₋₃ alkyl optionally substituted with OH, R⁷ is the formula (VI) or (VIII), R^{7a} is C₁₋₃ alkyl, Y is 1,4-phenylene, L is a bond, and Z is the formula (XI)-1.

Specific examples of compounds included in the present invention in an embodiment, include the following compounds.

A compound or a salt thereof selected from the group consisting of
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide (hereinafter sometimes referred to as “compound A”),
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-[(1R)-2-hydroxy-1-{4-[4-(hydroxymethyl)-1,3-thiazol-5-yl]phenyl}ethyl]-L-prolinamide (hereinafter sometimes referred to as “compound B”),
(4R)-1-[(2S)-2-(4-{4-[({6-cyclopropyl-4-{(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(2-oxo-1,3-oxazolidin-3-yl)phenyl]ethyl}-L-prolinamide (hereinafter sometimes referred to as “compound C”),
(4R)-1-[(2S)-2-(4-{4-[({6-cyclopropyl-4-[(1S,4S)-2,5-diazabicyclo[2.2.1]heptan-2-yl]-2-{[1-(2,2-difluoroethyl) piperidin-4-yl]oxy}-7-(6-fluoro-5-methyl-1H-indazol-4-yl)quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide (hereinafter sometimes referred to as “compound D”),
(4R)-1-[(2)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(1-methyl-1H-pyrazol-5-yl)phenyl]ethyl}-L-prolinamide (hereinafter sometimes referred to as “compound E”),
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-N-{(1R)-1-[4-(1-ethyl-1H-pyrazol-5-yl)phenyl]-2-hydroxyethyl}-4-hydroxy-L-prolinamide (hereinafter sometimes referred to as “compound F”),
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(2S)-2-methoxypropoxy]quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide (hereinafter sometimes referred to as “compound G”), and
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(2S)-2-methoxypropoxy]quinazolin-8-yl}oxy)methyl]phenyl}-1H-1,2,3-triazol-1-yl)-3-methylbutanoyl]-N-{(1R)-1-[4-(1-ethyl-1H-pyrazol-5-yl)phenyl]-2-hydroxyethyl}-4-hydroxy-L-prolinamide (hereinafter sometimes referred to as “compound H”).

Specific examples of compounds included in the present invention in an embodiment, include the following compounds.

A compound or a salt thereof selected from the group consisting of
(4R)-1-[(2S)-2-(4-{4-[{(7M)-6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide (hereinafter sometimes referred to as “compound A-1”),
(4R)-1-[(2S)-2-(4-{4-[({(7M)-6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl)-1H-1,2,3-triazol-1-yl)-3-methylbutanoyl]-4-hydroxy-N-[(1R)-2-hydroxy-1-{4-[4-(hydroxymethyl)-1,3-thiazol-5-yl]phenyl}ethyl]-L-prolinamide (hereinafter sometimes referred to as “compound B-1”),
(4R)-1-[(2S)-2-(4-{4-[({(7M)-6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(2-oxo-1,3-oxazolidin-3-yl)phenyl]ethyl}-L-prolinamide (hereinafter sometimes referred to as “compound C-1”),
(4R)-1-[(2S)-2-(4-{4-[({(7M)-6-cyclopropyl-4-[(1S,4S)-2,5-diazabicyclo[2.2.1]heptan-2-yl]-2-{[1-(2,2-difluoroethyl) piperidin-4-yl]oxy}-7-(6-fluoro-5-methyl-1H-indazol-4-yl)quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide (hereinafter sometimes referred to as “compound D-1”),
(4R)-1-[(2S)-2-(4-{4-[({(7M)-6-cyclopropyl-4-[{1S, 4S)-2,5-diazabicyclo[2.2.1]heptan-2-yl]-7-(6-fluoro-5-methyl-1H-indazol-4-yl)-2-[(oxan-4-yl)oxy]quinazolin-8-yl)oxy)methyl]phenyl}-1H-1,2,3-triazol-1-yl)-3-methylbutanoyl]-4-hydroxy-N-{(1R)-2-hydroxy-1-[4-(1-methyl-1H-pyrazol-5-yl)phenyl]ethyl}-L-prolinamide (hereinafter sometimes referred to as “compound E-1”),
(4R)-1-[(2S)-2-(4-{4-[({(7M)-6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-N-{(1R)-1-[4-(1-ethyl-1H-pyrazol-5-yl)phenyl]-2-hydroxyethyl}-4-hydroxy-L-prolinamide (hereinafter sometimes referred to as “compound F-1”),
(4R)-1-[(2S)-2-(4-{4-[({(7M)-6-cyclopropyl-4-[{1S, 4S)-2,5-diazabicyclo[2.2.1]heptan-2-yl]-7-(6-fluoro-5-methyl-1H-indazol-4-yl)-2-[(2S)-2-methoxypropoxy]quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl)-L-prolinamide (hereinafter sometimes referred to as “compound G-1”), and
(4R)-1-[(2S)-2-(4-{4-[({(7M)-6-cyclopropyl-4-[(1S,4S)-2,5-diazabicyclo[2.2.1]heptan-2-yl]-7-(6-fluoro-5-methyl-1H-indazol-4-yl)-2-[(2S)-2-methoxypropoxy]quinazolin-8-yl}oxy)methyl]phenyl}-1H-1,2,3-triazol-1-yl)-3-methylbutanoyl]-N-{(1R)-1-[4-(1-ethyl-1H-pyrazol-5-yl)phenyl]-2-hydroxyethyl}-4-hydroxy-L-prolinamide (hereinafter sometimes referred to as “compound H-1”).

Specific examples of compounds included in the present invention in an embodiment, include the following compounds.

A compound or a salt thereof selected from the group consisting of
(4R)-1-[(2S)-2-(4-{4-[({(7P)-6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide (hereinafter sometimes referred to as “compound A-2”),
(4R)-1-[(2S)-2-(4-{4-[({(7P)-6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-[(1R)-2-hydroxy-1-{4-[4-(hydroxymethyl)-1,3-thiazol-5-yl]phenyl}ethyl]-L-prolinamide (hereinafter sometimes referred to as “compound B-2”),
(4R)-1-[(2S)-2-(4-{4-[({(7P)-6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(2-oxo-1,3-oxazolidin-3-yl)phenyl]ethyl}-L-prolinamide (hereinafter sometimes referred to as “compound C-2”),
(4R)-1-[(2S)-2-(4-{4-[({(7P)-6-cyclopropyl-4-[(1S,4S)-2,5-diazabicyclo[2.2.1]heptan-2-yl]-2-{[1-(2,2-difluoroethyl) piperidin-4-yl]oxy}-7-(6-fluoro-5-methyl-1H-indazol-4-yl)quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide (hereinafter sometimes referred to as “compound D-2”),
(4R)-1-[(2S)-2-(4-{4-[{(7P)-6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(1-methyl-1H-pyrazol-5-yl)phenyl]ethyl}-L-prolinamide (hereinafter sometimes referred to as “compound E-2”),
(4R)-1-[(2S)-2-(4-{4-[({(7P)-6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-N-{(1R)-1-[4-(1-ethyl-1H-pyrazol-5-yl)phenyl]-2-hydroxyethyl}-4-hydroxy-L-prolinamide (hereinafter sometimes referred to as “compound F-2”),
(4R)-1-[(2S)-2-(4-{4-[({(7P)-6-cyclopropyl-4-[(1S,4S)-2,5-diazabicyclo[2.2.1]heptan-2-yl]-7-(6-fluoro-5-methyl-1H-indazol-4-yl)-2-[(2S)-2-methoxypropoxy]quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide (hereinafter sometimes referred to as “compound G-2”), and
(4R)-1-[(2S)-2-(4-{4-[({(7P)-6-cyclopropyl-4-[(1S,4S)-2,5-diazabicyclo[2.2.1]heptan-2-yl]-7-(6-fluoro-5-methyl-1H-indazol-4-yl)-2-[(2S)-2-methoxypropoxy]quinazolin-8-yl}oxy)methyl]phenyl}-1H-1,2,3-triazol-1-yl)-3-methylbutanoyl]-N-{(1R)-1-[4-(1-ethyl-1H-pyrazol-5-yl)phenyl]-2-hydroxyethyl}-4-hydroxy-L-prolinamide (hereinafter sometimes referred to as “compound H-2”).

Embodiments of the present invention will be shown below.
(17-1) A pharmaceutical composition for treating a cancer of colorectal cancer and/or lung cancer, the pharmaceutical composition comprising a compound selected from the group consisting of the compound A, the compound B, the compound C, the compound D, the compound E, the compound F, the compound G, and the compound H or a salt thereof. In another embodiment, a pharmaceutical composition for treating a cancer of colorectal cancer and/or lung cancer, the pharmaceutical composition comprising a compound selected from the group consisting of the compound A, the compound B, the compound C, the compound D, the compound E, the compound F, the compound G, and the compound H or a salt thereof and one or more pharmaceutically acceptable excipients.
(17-2) A pharmaceutical composition for treating G12D mutant KRAS-positive colorectal cancer and/or lung cancer, the pharmaceutical composition comprising a compound selected from the group consisting of the compound A, the compound B, the compound C, the compound D, the compound E, the compound F, the compound G, and the compound H or a salt thereof. In another embodiment, a pharmaceutical composition for treating G12D mutant KRAS-positive colorectal cancer and/or lung cancer, the pharmaceutical composition comprising a compound selected from the group consisting of the compound A, the compound B, the compound C, the compound D, the compound E, the compound F, the compound G, and the compound H or a salt thereof and one or more pharmaceutically acceptable excipients.
(17-3) A pharmaceutical composition for treating a cancer of colorectal cancer and/or lung cancer, the pharmaceutical composition comprising a compound selected from the group consisting of the compound A-1, the compound B-1, the compound C-1, the compound D-1, the compound E-1, the compound F-1, the compound G-1, and the compound H-1 or a salt thereof. In another embodiment, a pharmaceutical composition for treating a cancer of colorectal cancer and/or lung cancer, the pharmaceutical composition comprising a compound selected from the group consisting of the compound A-1, the compound B-1, the compound C-1, the compound D-1, the compound E-1, the compound F-1, the compound G-1, and the compound H-1 or a salt thereof and one or more pharmaceutically acceptable excipients.
(17-4) A pharmaceutical composition for treating G12D mutant KRAS-positive colorectal cancer and/or lung cancer, the pharmaceutical composition comprising a compound selected from the group consisting of the compound A-1, the compound B-1, the compound C-1, the compound D-1, the compound E-1, the compound F-1, the compound G-1, and the compound H-1 or a salt thereof. In another embodiment, a pharmaceutical composition for treating G12D mutant KRAS-positive colorectal cancer and/or lung cancer, the pharmaceutical composition comprising a compound selected from the group consisting of the compound A-1, the compound B-1, the compound C-1, the compound D-1, the compound E-1, the compound F-1, the compound G-1, and the compound H-1 or a salt thereof and one or more pharmaceutically acceptable excipients.
(17-5) A pharmaceutical composition for treating a cancer of colorectal cancer and/or lung cancer, the pharmaceutical composition comprising a compound selected from the group consisting of the compound A-2, the compound B-2, the compound C-2, the compound D-2, the compound E-2, the compound F-2, the compound G-2, and the compound H-2 or a salt thereof. In another embodiment, a pharmaceutical composition for treating a cancer of colorectal cancer and/or lung cancer, the pharmaceutical composition comprising a compound selected from the group consisting of the compound A-2, the compound B-2, the compound C-2, the compound D-2, the compound E-2, the compound F-2, the compound G-2, and the compound H-2 or a salt thereof and one or more pharmaceutically acceptable excipients.
(17-6) A pharmaceutical composition for treating G12D mutant KRAS-positive colorectal cancer and/or lung cancer, the pharmaceutical composition comprising a compound selected from the group consisting of the compound A-2, the compound B-2, the compound C-2, the compound D-2, the compound E-2, the compound F-2, the compound G-2, and the compound H-2 or a salt thereof. In another embodiment, a pharmaceutical composition for treating G12D mutant KRAS-positive colorectal cancer and/or lung cancer, the pharmaceutical composition comprising a compound selected from the group consisting of the compound A-2, the compound B-2, the compound C-2, the compound D-2, the compound E-2, the compound F-2, the compound G-2, and the compound H-2 or a salt thereof and one or more pharmaceutically acceptable excipients.
(17-7) A pharmaceutical composition for treating a cancer of colorectal cancer and/or lung cancer, the pharmaceutical composition comprising a compound selected from the group consisting of the compound A, the compound B, the compound C, the compound D, the compound E, and the compound F, or a salt thereof. In another embodiment, a pharmaceutical composition for treating a cancer of colorectal cancer and/or lung cancer, the pharmaceutical composition comprising a compound selected from the group consisting of the compound A, the compound B, the compound C, the compound D, the compound E, and the compound F or a salt thereof and one or more pharmaceutically acceptable excipients.
(17-8) A pharmaceutical composition for treating G12D mutant KRAS-positive colorectal cancer and/or lung cancer, the pharmaceutical composition comprising a compound selected from the group consisting of the compound A, the compound B, the compound C, the compound D, the compound E, and the compound F or a salt thereof. In another embodiment, a pharmaceutical composition for treating G12D mutant KRAS-positive colorectal cancer and/or lung cancer, the pharmaceutical composition comprising a compound selected from the group consisting of the compound A, the compound B, the compound C, the compound D, the compound E, and the compound F or a salt thereof and one or more pharmaceutically acceptable excipients.
(18-1) Use of a compound selected from the group consisting of the compound A, the compound B, the compound C, the compound D, the compound E, the compound F, the compound G, and the compound H or a salt thereof for the manufacture of a pharmaceutical composition for treating a cancer of colorectal cancer and/or lung cancer.
(18-2) Use of a compound selected from the group consisting of the compound A, the compound B, the compound C, the compound D, the compound E, the compound F, the compound G, and the compound H or a salt thereof for the manufacture of a pharmaceutical composition for treating G12D mutant KRAS-positive colorectal cancer and/or lung cancer.
(18-3) Use of a compound selected from the group consisting of the compound A-1, the compound B-1, the compound C-1, the compound D-1, the compound E-1, the compound F-1, the compound G-1, and the compound H-1 or a salt thereof for the manufacture of a pharmaceutical composition for treating a cancer of colorectal cancer and/or lung cancer.
(18-4) Use of a compound selected from the group consisting of the compound A-1, the compound B-1, the compound C-1, the compound D-1, the compound E-1, the compound F-1, the compound G-1, and the compound H-1 or a salt thereof for the manufacture of a pharmaceutical composition for treating G12D mutant KRAS-positive colorectal cancer and/or lung cancer.
(18-5) Use of a compound selected from the group consisting of the compound A-2, the compound B-2, the compound C-2, the compound D-2, the compound E-2, the compound F-2, the compound G-2, and the compound H-2 or a salt thereof for the manufacture of a pharmaceutical composition for treating a cancer of colorectal cancer and/or lung cancer.
(18-6) Use of a compound selected from the group consisting of the compound A-2, the compound B-2, the compound C-2, the compound D-2, the compound E-2, the compound F-2, the compound G-2, and the compound H-2 or a salt thereof for the manufacture of a pharmaceutical composition for treating G12D mutant KRAS-positive colorectal cancer and/or lung cancer.
(18-7) Use of a compound selected from the group consisting of the compound A, the compound B, the compound C, the compound D, the compound E, and the compound F or a salt thereof for the manufacture of a pharmaceutical composition for treating a cancer of colorectal cancer and/or lung cancer.
(18-8) Use of a compound selected from the group consisting of the compound A, the compound B, the compound C, the compound D, the compound E, and the compound F or a salt thereof for the manufacture of a pharmaceutical composition for treating G12D mutant KRAS-positive colorectal cancer and/or lung cancer.
(19-1) A compound selected from the group consisting of the compound A, the compound B, the compound C, the compound D, the compound E, the compound F, the compound G, and the compound H or a salt thereof for treating a cancer of colorectal cancer and/or lung cancer.
(19-2) A compound selected from the group consisting of the compound A, the compound B, the compound C, the compound D, the compound E, the compound F, the compound G, and the compound H or a salt thereof for treating G12D mutant KRAS-positive colorectal cancer and/or lung cancer.
(19-3) A compound selected from the group consisting of the compound A-1, the compound B-1, the compound C-1, the compound D-1, the compound E-1, the compound F-1, the compound G-1, and the compound H-1 or a salt thereof for treating a cancer of colorectal cancer and/or lung cancer.
(19-4) A compound selected from the group consisting of the compound A-1, the compound B-1, the compound C-1, the compound D-1, the compound E-1, the compound F-1, the compound G-1, and the compound H-1 or a salt thereof for treating G12D mutant KRAS-positive colorectal cancer and/or lung cancer.
(19-5) A compound selected from the group consisting of the compound A-2, the compound B-2, the compound C-2, the compound D-2, the compound E-2, the compound F-2, the compound G-2, and the compound H-2 or a salt thereof for treating a cancer of colorectal cancer and/or lung cancer.
(19-6) A compound selected from the group consisting of the compound A-2, the compound B-2, the compound C-2, the compound D-2, the compound E-2, the compound F-2, the compound G-2, and the compound H-2 or a salt thereof for treating G12D mutant KRAS-positive colorectal cancer and/or lung cancer.
(19-7) A compound selected from the group consisting of the compound A, the compound B, the compound C, the compound D, the compound E, and the compound F or a salt thereof for treating a cancer of colorectal cancer and/or lung cancer.
(19-8) A compound selected from the group consisting of the compound A, the compound B, the compound C, the compound D, the compound E, and the compound F or a salt thereof for treating G12D mutant KRAS-positive colorectal cancer and/or lung cancer.
(20-1) A compound selected from the group consisting of the compound A, the compound B, the compound C, the compound D, the compound E, the compound F, the compound G, and the compound H or a salt thereof for use in the treatment of a cancer of colorectal cancer and/or lung cancer.
(20-2) A compound selected from the group consisting of the compound A, the compound B, the compound C, the compound D, the compound E, the compound F, the compound G, and the compound H or a salt thereof for use in the treatment of G12D mutant KRAS-positive colorectal cancer and/or lung cancer.
(20-3) A compound selected from the group consisting of the compound A-1, the compound B-1, the compound C-1, the compound D-1, the compound E-1, the compound F-1, the compound G-1, and the compound H-1 or a salt thereof for use in the treatment of a cancer of colorectal cancer and/or lung cancer.
(20-4) A compound selected from the group consisting of the compound A-1, the compound B-1, the compound C-1, the compound D-1, the compound E-1, the compound F-1, the compound G-1, and the compound H-1 or a salt thereof for use in the treatment of G12D mutant KRAS-positive colorectal cancer and/or lung cancer.
(20-5) A compound selected from the group consisting of the compound A-2, the compound B-2, the compound C-2, the compound D-2, the compound E-2, the compound F-2, the compound G-2, and the compound H-2 or a salt thereof for use in the treatment of a cancer of colorectal cancer and/or lung cancer.
(20-6) A compound selected from the group consisting of the compound A-2, the compound B-2, the compound C-2, the compound D-2, the compound E-2, the compound F-2, the compound G-2, and the compound H-2 or a salt thereof for use in the treatment of G12D mutant KRAS-positive colorectal cancer and/or lung cancer.
(20-7) A compound selected from the group consisting of the compound A, the compound B, the compound C, the compound D, the compound E, and the compound F or a salt thereof for use in the treatment of a cancer of colorectal cancer and/or lung cancer.
(20-8) A compound selected from the group consisting of the compound A, the compound B, the compound C, the compound D, the compound E, and the compound F or a salt thereof for use in the treatment of G12D mutant KRAS-positive colorectal Cancer and/or lung cancer.
(21-1) Use of a compound selected from the group consisting of the compound A, the compound B, the compound C, the compound D, the compound E, the compound F, the compound G, and the compound H or a salt thereof for treating a cancer of colorectal cancer and/or lung cancer.
(21-2) Use of a compound selected from the group consisting of the compound A, the compound B, the compound C, the compound D, the compound E, the compound F, the compound G, and the compound H or a salt thereof for treating G12D mutant KRAS-positive colorectal cancer and/or lung cancer.
(21-3) Use of a compound selected from the group consisting of the compound A-1, the compound B-1, the compound C-1, the compound D-1, the compound E-1, the compound F-1, the compound G-1, and the compound H-1 or a salt thereof for treating a cancer of colorectal cancer and/or lung cancer.
(21-4) Use of a compound selected from the group consisting of the compound A-1, the compound B-1, the compound C-1, the compound D-1, the compound E-1, the compound F-1, the compound G-1, and the compound H-1 or a salt thereof for treating G12D mutant KRAS-positive colorectal cancer and/or lung cancer.
(21-5) Use of a compound selected from the group consisting of the compound A-2, the compound B-2, the compound C-2, the compound D-2, the compound E-2, the compound F-2, the compound G-2, and the compound H-2 or a salt thereof for treating a cancer of colorectal cancer and/or lung cancer.
(21-6) Use of a compound selected from the group consisting of the compound A-2, the compound B-2, the compound C-2, the compound D-2, the compound E-2, the compound F-2, the compound G-2, and the compound H-2 or a salt thereof for treating G12D mutant KRAS-positive colorectal cancer and/or lung cancer.
(21-7) Use of a compound selected from the group consisting of the compound A, the compound B, the compound C, the compound D, the compound E, and the compound F or a salt thereof for treating a cancer of colorectal cancer and/or lung cancer.
(21-8) Use of a compound selected from the group consisting of the compound A, the compound B, the compound C, the compound D, the compound E, and the compound F or a salt thereof for treating G12D mutant KRAS-positive colorectal cancer and/or lung cancer.
(22-1) A method for treating a cancer of colorectal cancer and/or lung cancer, the method including administering an effective amount of a compound selected from the group consisting of the compound A, the compound B, the compound C, the compound D, the compound E, the compound F, the compound G, and the compound H or a salt thereof to a subject.
(22-2) A method for treating G12D mutant KRAS-positive colorectal cancer and/or lung cancer, the method including administering an effective amount of a compound selected from the group consisting of the compound A, the compound B, the compound C, the compound D, the compound E, the compound F, the compound G, and the compound H or a salt thereof to a subject.
(22-3) A method for treating a cancer of colorectal cancer and/or lung cancer, the method including administering an effective amount of a compound selected from the group consisting of the compound A-1, the compound B-1, the compound C-1, the compound D-1, the compound E-1, the compound F-1, the compound G-1, and the compound H-1 or a salt thereof to a subject.
(22-4) A method for treating G12D mutant KRAS-positive colorectal cancer and/or lung cancer, the method including administering an effective amount of a compound selected from the group consisting of the compound A-1, the compound B-1, the compound C-1, the compound D-1, the compound E-1, the compound F-1, the compound G-1, and the compound H-1 or a salt thereof to a subject.
(22-5) A method for treating a cancer of colorectal cancer and/or lung cancer, the method including administering an effective amount of a compound selected from the group consisting of the compound A-2, the compound B-2, the compound C-2, the compound D-2, the compound E-2, the compound F-2, the compound G-2, and the compound H-2 or a salt thereof to a subject.
(22-6) A method for treating G12D mutant KRAS-positive colorectal cancer and/or lung cancer, the method including administering an effective amount of a compound selected from the group consisting of the compound A-2, the compound B-2, the compound C-2, the compound D-2, the compound E-2, the compound F-2, the compound G-2, and the compound H-2 or a salt thereof to a subject.
(22-7) A method for treating a cancer of colorectal cancer and/or lung cancer, the method including administering an effective amount of a compound selected from the group consisting of the compound A, the compound B, the compound C, the compound D, the compound E, and the compound F or a salt thereof to a subject.
(22-8) A method for treating G12D mutant KRAS-positive colorectal cancer and/or lung cancer, the method including administering an effective amount of a compound selected from the group consisting of the compound A, the compound B, the compound C, the compound D, the compound E, and the compound F or a salt thereof to a subject.

The compound of the formula (I) may have tautomers or geometrical isomers depending on the type of the substituent. In this specification, the compound of the formula (I) is sometimes described only as one of isomers, but the present invention includes isomers other than the above one, and includes separated isomers or mixtures thereof.

In addition, the compound of the formula (I) may have an asymmetric carbon atom or an axial chirality and may have diastereomers based on them. The present invention includes separated diastereomers of the compound of the formula (I) or mixtures thereof.

Furthermore, the present invention includes pharmaceutically acceptable prodrugs of the compound represented by the formula (I). A pharmaceutically acceptable prodrug is a compound having a group that can be converted into an amino group, a hydroxy group, a carboxy group, or the like by solvolysis or under physiological conditions. Examples of groups to form a prodrug include groups described in Prog. Med., 1985, 5, p. 2157-2161 or in “Iyakuhin no Kaihatsu (development of pharmaceuticals)”, Vol. 7, Bunshisekkei (molecular design), Hirokawa Shoten, 1990, p. 163-198.

In addition, the salt of the compound of the formula (I) is a pharmaceutically acceptable salt of the compound of the formula (I), and may be an acid addition salt or a salt formed with a base depending on the type of the substituent. Examples thereof include salts shown in P. Heinrich Stahl, Handbook of Pharmaceutical Salts Properties, Selection, and Use, Wiley-VCH, 2008. Specific examples include an acid addition salt with an inorganic acid, such as hydrochloric acid, hydrobromic acid, hydroiodic acid, sulfuric acid, nitric acid, or phosphoric acid, or with an organic acid, such as formic acid, acetic acid, propionic acid, oxalic acid, malonic acid, succinic acid, fumaric acid, maleic acid, lactic acid, malic acid, mandelic acid, tartaric acid, dibenzoiltartaric acid, ditoluoyltartaric acid, citric acid, methanesulfonic acid, ethanesulfonic acid, benzenesulfonic acid, p-toluenesulfonic acid, aspartic acid, or glutamic acid, a salt with an inorganic metal, such as sodium, potassium, magnesium, calcium, or aluminum, a salt with an organic base, such as methylamine, ethylamine, or ethanolamine, a salt with various amino acids and amino acid derivatives, such as acetylleucine, lysine, and ornithine, and an ammonium salt.

Furthermore, the present invention also includes various hydrates, solvates, and crystal polymorphism substances of the compound of the formula (I) and a salt thereof. In addition, the present invention also includes compounds labeled with various radioactive or non-radioactive isotopes.

### (Production Method)

The compound of the formula (I) and a salt thereof can be produced by applying various known synthetic methods using characteristics based on the basic structure or the type of substituent thereof. Here, depending on the type of functional group, it is sometimes effective as a production technique to substitute the functional group with an appropriate protective group (a group that can be easily converted to the functional group) in the process from a raw material to an intermediate. Examples of the protective group include protective groups described in P. G. M. Wuts and T. W. Greene, “Greene's Protective Groups in Organic Synthesis”, 5th edition, John Wiley & Sons Inc., 2014, and a group appropriately selected from the protective groups is used depending on the reaction conditions. In such a method, a reaction is carried out with the protective group introduced, and then the protective group is removed, as required, whereby a desired compound can be obtained.

In addition, a prodrug of the compound of the formula (I) can be produced by introducing a special group in a process from a raw material to an intermediate as for the above protective group, or by further carrying out a reaction using the compound of the formula (I) obtained. This reaction can be carried out by applying a method known to a parson skilled in the art, such as common esterification, amidation, or dehydration.

A typical method for producing the compound of the formula (I) will be described below. The production method can also be carried out with reference to a reference attached to the description. Note that the production method of the present invention is not limited to the examples described below.

In this specification, the following abbreviations are sometimes used.

DMF: N, N-dimethylformamide, DMAc: N, N-dimethylacetamide, THF: tetrahydrofuran, MeCN: acetonitrile, MeOH: methanol, EtOH: ethanol, DOX: 1,4-dioxane, DMSO: dimethyl sulfoxide, TEA: triethylamine, DIPEA: N, N-diisopropylethylamine, tBuOK: potassium tert-butoxide, PdCl₂ (dppf)·CH₂Cl₂: [1,1′-bis(diphenylphosphino)ferrocene]palladium(II) dichloridedichloromethane adduct, Pd/C: palladium on carbon.

### (Production Method 1)

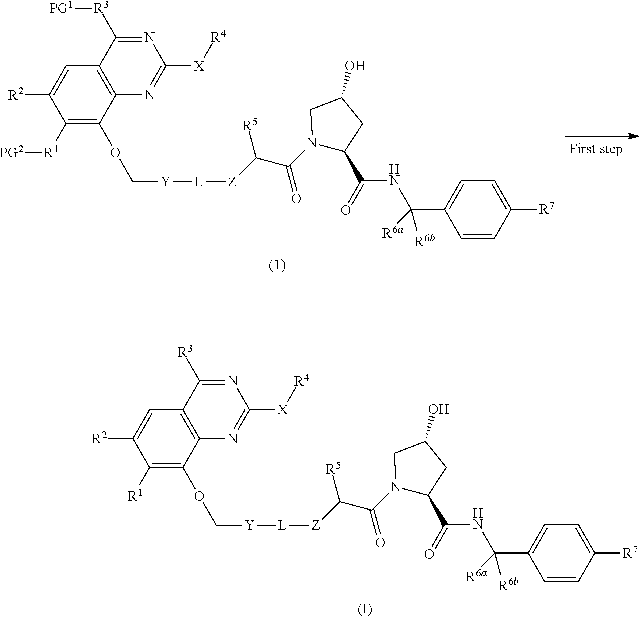

(In the formula, PG¹ represents a protective group and PG² represents a protective group or a hydrogen atom.)

The compound of the formula (I) can be obtained by subjecting a compound (1) to a deprotection reaction. Here, examples of the protective group include a tert-butoxycarbonyl group, a benzyl group, a triphenylmethyl group, a benzyloxycarbonyl group, a tert-butyl(dimethyl)silyl group, a (trimethylsilyl)ethoxymethyl group, a trifluoroacetyl group, an allyl group, a tetrahydro-2H-pyran-2-yl group, and a methoxymethyl group.

This reaction is performed by stirring, from under cooling to reflux with heat, generally for 0.1 hours to 5 days. A solvent used here is not particularly limited, and examples thereof include an alcohol, such as MeOH or EtOH, an aromatic hydrocarbon, such as benzene, toluene, or xylene, a halogenated hydrocarbon, such as dichloromethane, 1,2-dichloroethane, or chloroform, an ether, such as diethyl ether, THF, DOX, or dimethoxyethane, DMF, DMSO, ethyl acetate, MeCN, or water, and a mixture thereof. A deprotection reagent is selected depending on the type of the protective group, and examples thereof include, but are not particularly limited to, a base, such as an aqueous sodium hydroxide solution, an aqueous potassium hydroxide solution, or an aqueous lithium hydroxide solution, an acid, such as hydrochloric acid or trifluoroacetic acid, and tetra-n-butylammonium fluoride. Performing the reaction in the presence of triisopropylsilane is sometimes advantageous for smoothly promoting the reaction.

For example, the following can be referred as a reference about this reaction.

P. G. M. Wuts and T. W. Greene, “Greene's Protective Groups in Organic Synthesis”, 5th edition, John Wiley & Sons Inc., 2014

Note that when the compound (1) as a raw material has an axial chirality, a stereoisomer which is obtained by once separating the compound (1) may be used for this reaction.

In addition, the compound of the formula (I) or an intermediate thereof sometimes has an axial chirality and is obtained as a mixture of diastereomers, and each diastereomer can be isolated by separation using a common separation operation, for example, ODS column chromatography or silica gel column chromatography.

By subjecting the compound of the formula (I) to the following operation, the hydrochloride salt of the compound of the formula (I) can be obtained.

The compound of the formula (I) is dissolved in CH₂Cl₂ and MeOH, hydrogen chloride (4M DOX solution, 10 equivalents) is added under ice-bath cooling, and the mixture is stirred under ice-bath cooling for 30 minutes. This reaction mixture is concentrated under reduced pressure, diethyl ether is added to the resulting residue, and the produced solid is filtered and dried under reduced pressure to give the hydrochloride salt of the compound of the formula (I).

By subjecting the hydrochloride salt of the compound of the formula (I) to the following operation, free form of the compound of the formula (I) can be obtained.

The hydrochloride salt of the compound of the formula (I) is purified by ODS column chromatography (MeCN/0.1% aqueous formic acid solution), fractions containing the target compound are collected and basified with saturated aqueous sodium hydrogen carbonate solution, and then the solution is extracted with CHCl₃/MeOH (5/1). The combined organic layer is dried over anhydrous sodium sulfate, and the solution is concentrated under reduced pressure. The resulting solid is washed with diethyl ether and dried under reduced pressure to give the compound of the formula (I).

### (Raw Material Synthesis 1)

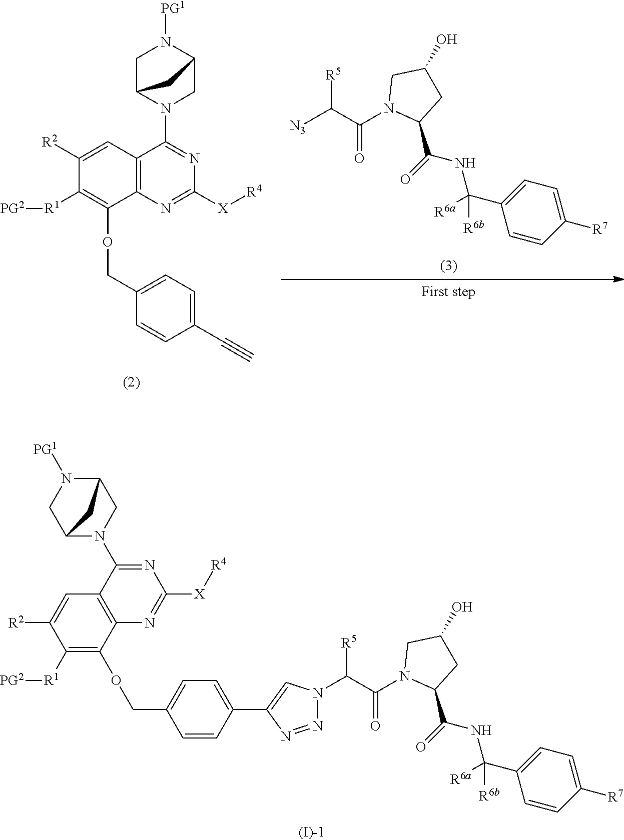

The production method is a first method for producing a compound (1)-1 included in the raw material compound (1).

### (First Step)

This step is a method for producing the compound (1)-1 by a cycloaddition reaction of a compound (2) and a compound (3).

In this reaction, the compound (2) and the compound (3) are used in an equal amount or with one compound thereof in a larger amount, and the mixture of the compounds is stirred preferably in the presence of a copper salt, further preferably in the presence of a copper salt and a reductant, in a solvent inactive for the reaction or with no solvent, from under cooling to under reflux with heat, preferably at 0° C. to 100° C., generally for 0.1 hours to 5 days. Examples of the solvent used here include, but are not particularly limited to, a halogenated hydrocarbon, such as dichloromethane, 1,2-dichloroethane, or chloroform, an aromatic hydrocarbon, such as benzene, toluene, or xylene, an ether, such as diethyl ether, THF, DOX, or 1,2-dimethoxyethane, DMF, DMSO, ethyl acetate, MeCN, tBuOH, water, and a mixture thereof. Examples of the copper salt include CuI, CuSO₄, and CuOTf. An example of the reductant is sodium ascorbate. Performing the reaction in the presence of TEA, DIPEA, N-methylmorpholine (NMM), 2,6-lutidine, tris[(1-benzyl-1H-1,2,3-triazol-4-yl)methyl]amine (TBTA), or the like is sometimes advantageous for smoothly promoting the reaction.

### REFERENCE

Angew. Chem. Int. Ed. 2002, 41, p. 2596-2599.

### (Raw Material Synthesis 2)

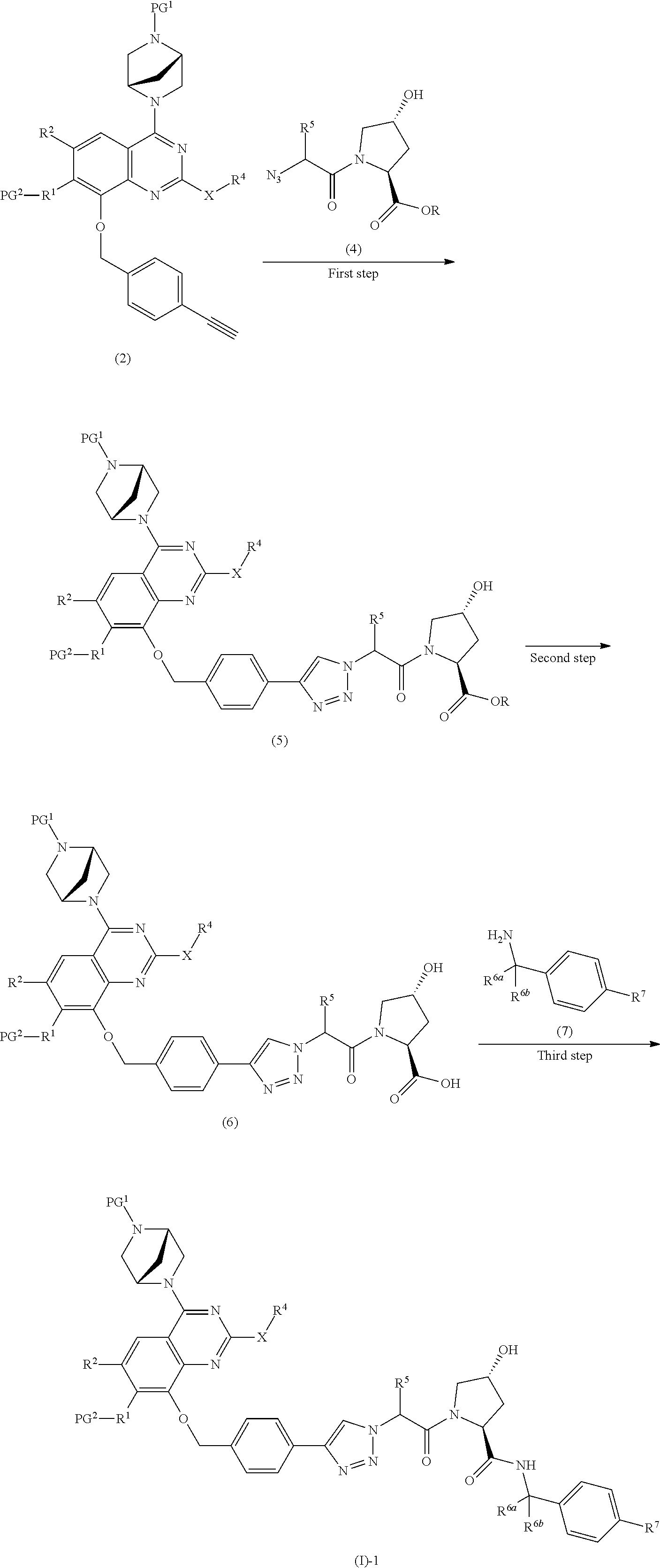

(In the formulae, R represents a C₁₋₃ alkyl group.)

This production method is a second method for producing the compound (1)-1 included in the raw material compound (1).

### (First Step)

This step is a method for producing a compound (5) by a cycloaddition reaction of the compound (2) and a compound (4).

The reaction conditions are the same as in the first step of the Raw Material Synthesis 1.

### (Second Step)

This step is a method for producing a compound (6) by hydrolysis of the compound (5).

This reaction is performed by stirring the compound (5) from under cooling to under reflux with heat generally for 0.1 hours to 5 days. Examples of the solvent used here include, but are not particularly limited to, an alcohol, acetone, N, N-dimethylformamide, and tetrahydrofuran. In addition, a mixed solvent of the above solvent and water is sometimes suitable for the reaction. Examples of the hydrolysis reagent include, but are not particularly limited to, an aqueous sodium hydroxide solution, an aqueous potassium hydroxide solution, and trimethyltin hydroxide.

For example, the following can be referred as a reference about this reaction.
The Chemical Society of Japan, “Jikken Kagaku Koza (lectures on experimental chemistry) (5th edition)”, Vol. 16 (2005) (Maruzen)
Angew. Chem. Int. Ed. 2005, 44, p. 1378-1382.

### (Third Step)

This step is a method for producing the compound (1)-1 by an amidation reaction of the compound (6) and a compound (7).

In this reaction, the compound (6) and the compound (7) are used in an equal amount or with one compound thereof in a larger amount, the mixture of the compounds is stirred in the presence of a condensing agent, in a solvent inactive for the reaction, from under cooling to under heating, preferably at −20° C. to 60° C., generally for 0.1 hours to 5 days. Examples of the solvent include, but are not particularly limited to, an aromatic hydrocarbon, such as toluene, an ether, such as THF or DOX, a halogenated hydrocarbon, such as dichloromethane, an alcohol, N, N-dimethylformamide, DMSO, ethyl acetate, MeCN, and a mixture thereof. Examples of the condensing agent include (benzotriazol-1-yloxy)tripyrrolidinophosphonium hexafluorophosphate (PyBOP), O-(7-azabenzotriazol-1-yl)-N,N,N′,N′-tetramethyluronium hexafluorophosphate (HATU), 1-(3-dimethylaminopropyl)-3-ethylcarbodiimide or the hydrochloride thereof, N,N′-dicyclohexylcarbodiimide (DCC), 1,1′-carbonyldiimidazole (CDI), and diphenylphosphoryl azide (DPPA). Use of an additive (for example, 1-hydroxybenzotriazole) is sometimes preferred for the reaction. Performing the reaction in the presence of an organic base, such as TEA, DIPEA, or NMM, or an inorganic base, such as potassium carbonate, sodium carbonate, or potassium hydroxide, is sometimes advantageous for smoothly promoting the reaction.

Alternatively, a method in which the compound (6) is converted into a reactive derivative, which is then subjected to an acylation reaction, can be used. Examples of the reactive derivative of a carboxylic acid include an acid halogenation product obtained by a reaction with a halogenating agent, such as phosphorus oxychloride or thionyl chloride, a mixed acid anhydride obtained by a reaction with isobutyl chloroformate, and an active ester obtained by condensation with 1-hydroxybenzotriazole or the like. The reaction of such a reactive derivative and the compound (7) can be performed in a solvent inactive for the reaction, such as a halogenated hydrocarbon, an aromatic hydrocarbon, or an ether, from under cooling to under heating, preferably at −20° C. to 120° C.

### REFERENCE

S. R. Sandler and W. Karo, “Organic Functional Group Preparations”, 2nd edition, Vol. 1, Academic Press Inc., 1991 The Chemical Society of Japan, “Jikken Kagaku Koza (lectures on experimental chemistry (5th edition)”, Vol. 16 (2005) (Maruzen)

### (Raw Material Synthesis 3)

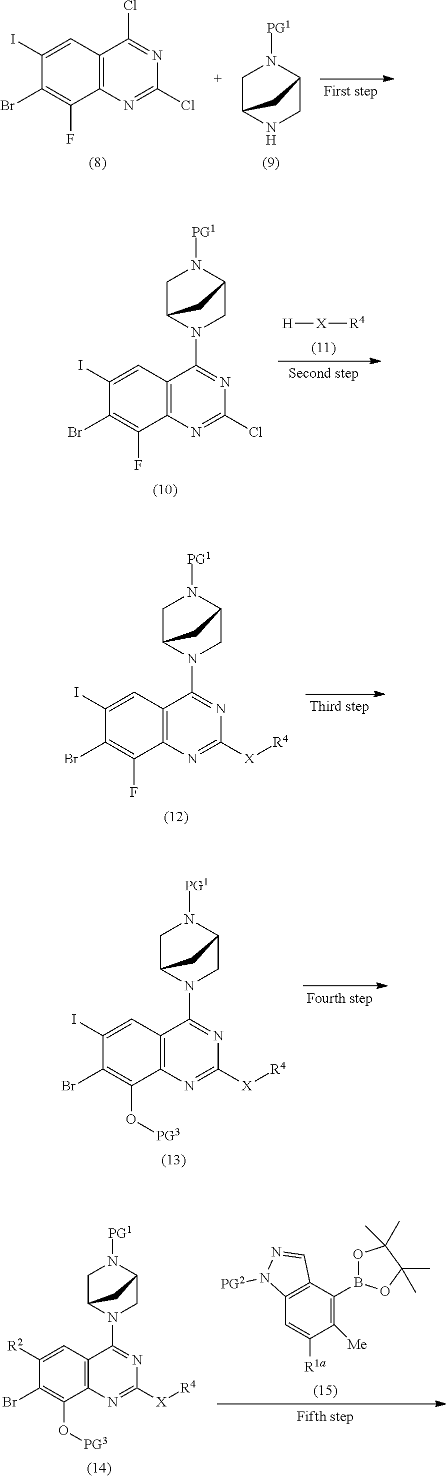

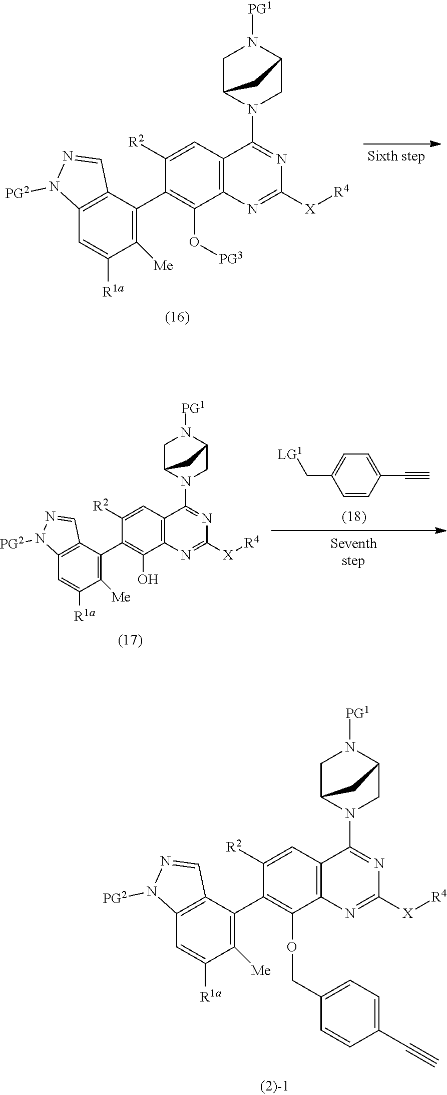

(In the formula, PG³ represents a protective group and LG¹ represents a leaving group.)

This production method is a first method for producing a raw material compound (2)-1.

### (First Step)

This step is a method for producing a compound (10) by an ipso substitution reaction of a compound (8) and a compound (9).

In this reaction, the compound (8) and the compound (9) are used in an equal amount or with one compound thereof in a larger amount, and the mixture of the compounds is stirred in a solvent inactive for the reaction or with no solvent, from under cooling to under reflux with heat, preferably at 0° C. to 80° C., generally for 0.1 hours to 5 days. Examples of the solvent used here include, but are not particularly limited to, a halogenated hydrocarbon, such as dichloromethane, 1,2-dichloroethane, or chloroform, an aromatic hydrocarbon, such as benzene, toluene, or xylene, an ether, such as diethyl ether, THF, DOX, or 1,2-dimethoxyethane, DMF, DMAC, DMSO, ethyl acetate, MeCN, and a mixture thereof. Performing the reaction in the presence of an organic base, such as TEA, DIPEA, N-methylmorpholine (NMM), 1,4-diazabicyclo[2.2.2]octane (DABCO), or tBuOK, or an inorganic base, such as sodium hydroxide, potassium carbonate, sodium carbonate, or cesium carbonate, is sometimes advantageous for smoothly promoting the reaction.

### (Second Step)

This step is a method for producing a compound (12) by an ipso substitution reaction of the compound (10) and a compound (11).

The reaction conditions are the same as in the first step of the Raw Material Synthesis 3.

### (Third Step)

This step is a method for producing a compound (13) by an ipso substitution reaction of the compound (12) and PG³-OH.

Examples of the PG³-OH used here include benzyl alcohol and p-methoxybenzyl alcohol.

The reaction conditions are the same as in the first step of the Raw Material Synthesis 3.

### (Fourth Step)

This step is a method for producing a compound (14) by a Suzuki coupling reaction of the compound (13) and a boronic acid derivative containing an R² group. Examples of the boronic acid derivative used here include, but are not particularly limited to, boronic acid, a boronic acid ester, boronic acid pinacol ester, a triol borate salt, and a trifluoroboric acid salt.

In this reaction, the compound (13) and the boronic acid derivative containing an R² group are used in an equal amount or with one compound thereof in a larger amount, and the mixture of the compounds is stirred in a solvent inactive for the reaction, in the presence of a base and a palladium catalyst, from at room temperature to under reflux with heat, preferably at 20° C. to 140° C., generally for 0.1 hours to 5 days. Examples of the solvent used here include, but are not particularly limited to, a halogenated hydrocarbon, such as dichloromethane, 1,2-dichloroethane, or chloroform, an aromatic hydrocarbon, such as benzene, toluene, or xylene, an ether, such as diethyl ether, THF, DOX, or 1,2-dimethoxyethane, an alcohol, such as MeOH, EtOH, isopropyl alcohol, butanol, or amyl alcohol, DMF, DMSO, MeCN, 1,3-dimethylimidazolidin-2-one, water, and a mixture thereof. Examples of the base include inorganic bases, such as tripotassium phosphate, sodium carbonate, potassium carbonate, and sodium hydroxide. Examples of the palladium catalyst include tetrakis(triphenylphosphine)palladium, bis(triphenylphosphine)palladium(II) dichloride, [1,1′-bis(diphenylphosphino)ferrocene]palladium(II) dichloride dichloromethane additive, (1E,4E)-1,5-diphenylpenta-1,4-dien-3-one/palladium (3:2), and (2-dicyclohexylphosphino-2′,6′-diisopropoxy-1,1′-biphenyl) [2-(2′-amino-1,1′-biphenyl)]palladium(II) methanesulfonate. Performing the reaction in the presence of a ligand, such as dicyclohexyl(2′,6′-dimethoxybiphenyl-2-yl)phosphine or dicyclohexyl(2′,6′-diisopropoxy-[1,1′-biphenyl]-2-yl)phosphine, is sometimes advantageous for smoothly promoting the reaction. In addition, heating the mixture by microwave irradiation is sometimes advantageous for smoothly promoting the reaction.

### REFERENCE

J. Am. Chem. Soc., 2005, 127, p. 4685-4696

### (Fifth Step)

This step is a method for producing a compound (16) by a Suzuki coupling reaction of the compound (14) and a compound (15).

The reaction conditions are the same as in the fourth step of the Raw Material Synthesis 3.

### (Sixth Step)

This step is a method for producing a compound (17) by deprotection by a catalytic hydrogenation reaction of the compound (16).

This reaction can be performed by stirring the compound (16) under hydrogen atmosphere, from under normal pressure to under increased pressure, in a solvent inactive for the reaction, such as MeOH, EtOH, or ethyl acetate, in the presence of a metal catalyst, from under cooling to under heating, preferably at room temperature, for 1 hour to 5 days. As the metal catalyst, a palladium catalyst, such as Pd/C or palladium black, a platinum catalyst, such as a platinum plate or platinum oxide, or a nickel catalyst, such as reduced nickel or Raney nickel, is used.

### (Seventh Step)

This step is a method for producing the compound (2)-1 by a reaction of the compound (17) and a compound (18).

This reaction is performed by reacting a mixture of the compound (17) and the compound (18) in an equal amount or with one compound thereof in a larger amount in the presence of a base, in a solvent inactive for the reaction, from under cooling to under reflux with heat, preferably at 0° C. to 80° C., generally for 0.1 hours to 5 days. The solvent used here is not particularly limited, and examples thereof include an aromatic hydrocarbon, such as benzene, toluene, or xylene, an alcohol, such as MeOH or EtOH, an ether, such as diethyl ether, THF, DOX, or 1,2-dimethoxyethane, a halogenated hydrocarbon, such as dichloromethane, 1,2-dichloroethane, or chloroform, DMF, DMSO, ethyl acetate, MeCN, and a mixture thereof. Examples of the base include, but are not particularly limited to, an organic base, such as TEA, DIPEA, 1,8-diazabicyclo[5.4.0]-7-undecene, n-butyllithium, or tBuOK, and an inorganic base, such as sodium hydroxide, sodium carbonate, potassium carbonate, cesium carbonate, or sodium hydroxide. Performing the reaction in the presence of a phase transfer catalyst, such as tetra-n-butylammonium chloride, is sometimes advantageous.

For example, the following can be referred as a reference about this reaction.
The Chemical Society of Japan, “Jikken Kagaku Koza (lectures on experimental chemistry)”, 5th edition, Vol. 14, Maruzen, 2005

### (Raw Material Synthesis 4)

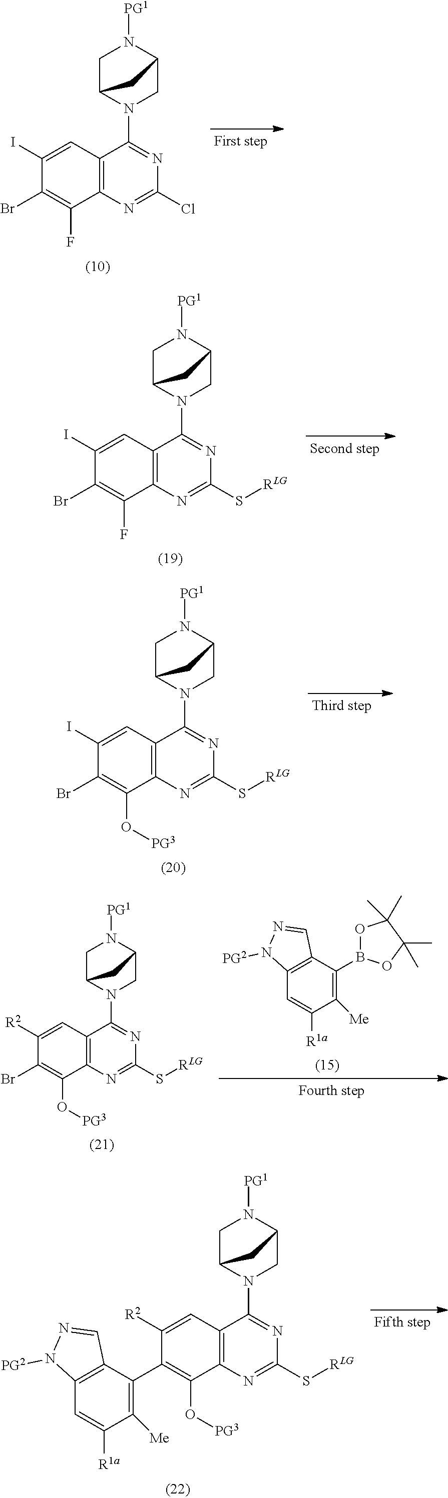

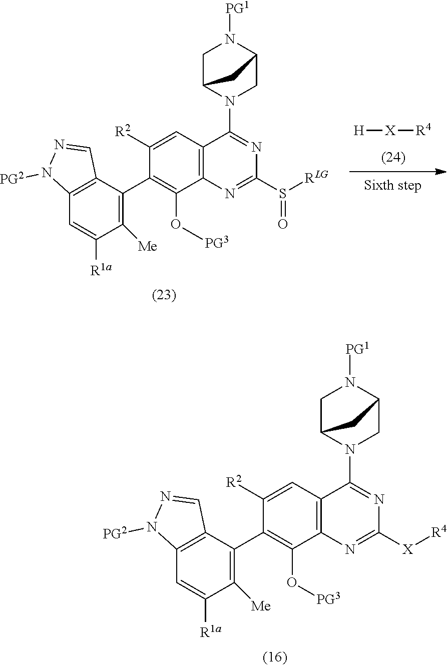

(In the formula, R^{LG} represents a C₁₋₁₂ alkyl group.)

This production method is a second method for producing the raw material compound (16).

### (First Step)

This step is a method for producing a compound (19) by an ipso substitution reaction of the compound (10) and R^{LG}—SH. Examples of the R^{LG}—SH used here include C₁₋₁₂ alkylthiols, for example, ethanethiol and dodecanethiol.

The reaction conditions are the same as in the first step of the Raw Material Synthesis 3.

### (Second Step)

This step is a method for producing a compound (20) by an ipso substitution reaction of the compound (19) and PG³-OH. Examples of the PG³-OH used here include benzyl alcohol and p-methoxybenzyl alcohol.

The reaction conditions are the same as in the first step of the Raw Material Synthesis 3.

### (Third Step)

This step is a method for producing a compound (21) by a Suzuki coupling reaction of the compound (20) and a boronic acid derivative containing an R² group.

The reaction conditions are the same as in the fourth step of the Raw Material Synthesis 3.

### (Fourth Step)

This step is a method for producing a compound (22) by a Suzuki coupling reaction of the compound (21) and the compound (15).

The reaction conditions are the same as in the fourth step of the Raw Material Synthesis 3.

### (Fifth Step)

This step is a method for producing a compound (23) by an oxidation reaction of the compound (22).

In this reaction, the compound (22) is treated with an oxidant in an equal amount or an excess amount in a solvent inactive for the reaction, from under cooling to under heating, preferably at −20° C. to 80° C., generally for 0.1 hours to 3 days. In this reaction, oxidation with m-chloroperbenzoic acid, perbenzoic acid, peracetic acid, sodium hypochlorite, or hydrogen peroxide is suitably used. Examples of the solvent include an aromatic hydrocarbon, an ether, a halogenated hydrocarbon such as dichloromethane, DMF, DMSO, ethyl acetate, MeCN, and a mixture thereof. Other examples of the oxidant include cumene hydroperoxide, Oxone, active manganese dioxide, chromic acid, potassium permanganate, and sodium periodate.

### REFERENCE

The Chemical Society of Japan, “Jikken Kagaku Koza (lectures on experimental chemistry)”, 5th edition, Vol. 17, Maruzen, 2005

### (Sixth Step))

This step is a method for producing the compound (16) by an ipso substitution reaction of the compound (23) and a compound (24).

The reaction conditions are the same as in the first step of the Raw Material Synthesis 3.

### (Raw Material Synthesis 5)

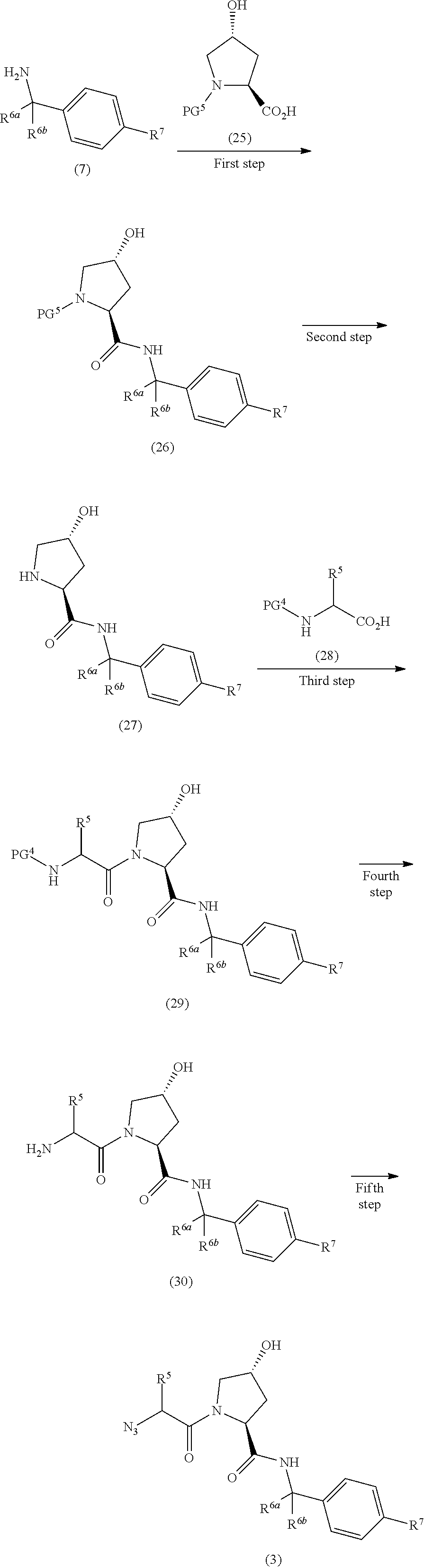

(In the formula, PG⁴ and PG⁵ represent a protective group.)

This production method is a method for producing the raw material compound (3).

### (First Step)

This step is a method for producing a compound (26) by an amidation reaction of the compound (7) and a compound (25).

The reaction conditions are the same as in the third step of the Raw Material Synthesis 2.

### (Second Step)

This step is a method for producing of a compound (27) by a deprotection reaction of the compound (26).

The reaction conditions are the same as in the step described in the Production Method 1.

### (Third Step)

This step is a method for producing a compound (29) by an amidation reaction of the compound (27) and a compound (28).

The reaction conditions are the same as in the third step of the Raw Material Synthesis 2.

### (Fourth Step)

This step is a method for producing of a compound (30) by a deprotection reaction of the compound (29).

The reaction conditions are the same as in the step described in the Production Method 1.

### (Fifth Step)

This step is a method for producing the compound (3) by a reaction of the compound (30) and a diazo-transfer reagent.

In this reaction, the compound (30) is treated with the diazo-transfer reagent in an equal amount or an excess amount in a solvent inactive for the reaction, from under cooling to under heating, preferably at 0° C. to 50° C., generally for 0.1 hours to 3 days. Examples of the diazo-transfer reagent include, but are not particularly limited to, trifluoromethanesulfonyl azide, imidazole-1-sulfonyl azide, or a salt thereof, and 2-azido-1,3-dimethylimidazolinium hexafluorophosphate (ADMP). Performing the reaction in the presence of an organic base, such as TEA, 4-dimethylaminopyridine (DMAP), or 2,6-lutidine, and a catalytic amount of a copper salt, such as CuSO₄, is sometimes advantageous. Examples of the solvent include a halogenated hydrocarbon, such as THF or dichloromethane, MeCN, an alcohol, water, and a mixture thereof.

### REFERENCE

J. Org. Chem. 2012, 77, p. 1760-1764 Nature 2019, 574, p. 86-89 Org. Biomol. Chem. 2014, 12, p. 4397-4406

The compound of the formula (I) is isolated and purified as a free compound, or a salt, hydrate, solvate, or crystal polymorphous substance thereof. A salt of the compound of the formula (I) can also be produced by subjecting the compound to a salt formation reaction which is an ordinary method.

The isolation and purification are performed by applying a common chemical operation, such as extraction, fractional crystallization, or various types of fraction chromatography.

Various types of isomers can be produced by selecting an appropriate raw material compound, or can be separated by using a difference in physiochemical properties between the isomers. For example, an optical isomer can be obtained by a general optical resolution method of a racemate (for example, fractional crystallization for inducing a racemate to a diastereomer salt with an optically active base or acid, or chromatography using a chiral column), and can also be produced from an appropriate optically active raw material compound.

The compound of the formula (I) according to the present invention can be used for treatment of a cancer of colorectal cancer and/or lung cancer, in particular, a G12D mutant KRAS-positive cancer.

A pharmaceutical composition that contains one or two or more compounds of the formula (I) or salts thereof as active ingredients can be prepared by a usually used method using an excipient usually used in the art, that is, a pharmaceutical excipient or a pharmaceutical carrier.

The administration may be either oral administration with a tablet, pill, capsule, granule, powder, liquid, or other agent or parenteral administration with an intraarticular, intravenous, intramuscular, or other injection, a transmucosal agent, or an inhalant.

As a solid composition for oral administration, a tablet, powder, granular, or other agent is used. In such a solid composition, one or two or more active ingredients are mixed with at least one inactive excipient. The composition may contain an inactive additive, for example, a lubricant, a disintegrator, a stabilizer, a dissolution aid according to an ordinary method. A tablet or pill may be coated with a sugar coating or a film soluble in the stomach or intestine, as needed.

Liquid compositions for oral administration include a pharmaceutically acceptable emulsion, solution, suspension, syrup, or elixir agent, and contain a generally used inactive diluent, for example, purified water or EtOH (ethanol). The liquid composition may contain, in addition to the inactive diluent, an adjuvant, such as a solubilizer, a wetting agent, or a suspending agent, a sweetening agent, a flavor, a fragrant, or a preservative.

The injection agents for parenteral administration include a sterile aqueous or nonaqueous solution, suspension, or emulsion agent. Examples of the aqueous solvent include distilled water for injection or physiological saline. An example of the nonaqueous solvent is an alcohol, such as EtOH. Such a composition may further contain an isotonizing agent, a preservative, a wetting agent, an emulsifier, a dispersant, a stabilizer, or a dissolution aid. These are sterilized, for example, by filtration through a bacteria keeping filter, incorporation of a microbicide, or irradiation. In addition, such a composition can be produced as a sterile solid composition, which is dissolved or suspended in sterile water or a sterile solvent for injection before use.

The transmucosal agent, such as an inhalant or a transnasal agent, is used in a solid, liquid, or semi-solid form, and can be produced according to a conventionally known method. For example, a known excipient, and in addition, a pH modifier, a preservative, a surfactant, a lubricant, a stabilizer, a thickener, or the like may be appropriately added. The administration can be performed by using an appropriate device for inhalation or insufflation. For example, the agent can be administered using a known device, such as a metering and administering inhalation device, or an atomizer, as a compound alone or a powder of a mixture formulated, or as a solution or a suspension in combination with a pharmaceutically acceptable carrier. A dry powder inhaler or the like may be for a single administration or multiple administrations, and dry powder or powder-containing capsule can be used. Alternatively, the agent may be used in a form of a pressurized aerosol spray or the like using an appropriate ejection agent, for example, a suitable gas, such as a chlorofluoroalkane or carbon dioxide.

In the case of a common oral administration, the daily dose is appropriately about 0.001 to 100 mg/kg body weight, preferably 0.1 to 30 mg/kg body weight, further preferably 0.1 to 10 mg/kg body weight, and the dose is given at once or is divided into two to four times in a day. In the case of intravenous administration, the daily dose is appropriately about 0.0001 to 10 mg/kg body weight, and is given at once or is divided into multiple times in a day. In addition, the daily dose of a transmucosal agent is about 0.001 to 100 mg/kg body weight, and is given at once or is divided into multiple times in a day. The dose is appropriately decided depending on the individual case taking the symptom, age, sex, and the like into account.

Depending on the route of administration, dosage form, site of administration, and types of excipient and additive, the pharmaceutical composition of the present invention contains 0.01 to 100% by weight, in an embodiment, 0.01 to 50% by weight, of one or more compounds of the formula (I) or salts thereof which are active ingredients.

The compound of the formula (I) can be used in combination with various therapeutic agents or preventive agents for a disease to which the compound of the formula (I) is considered to have an effectiveness. The combination use may be simultaneous administration, or separate administration either sequential or with a desired interval. A simultaneous administration preparation may be a formulated agent or may be separately formulated.

### EXAMPLES

The production method of the compound of the formula (I) will be described in further detail below based on examples. Note that the present invention is not to be limited to the compounds described in the following examples. The production methods of raw material compounds are also shown in the production examples. The production method of the compound of the formula (I) is not limited only to the production methods of specific examples described below, and the compound of the formula (I) can also be produced by a combination of the production methods or a method that is obvious to a person skilled in the art.

Note that, in this specification, a compound is sometimes named by using a naming soft, such as ACD/Name (registered tradename, Advanced Chemistry Development, Inc.).

For the purpose of convenience, the concentration mol/L is shown as M. For example, 1 M aqueous sodium hydroxide solution means an aqueous sodium hydroxide solution of 1 mol/L.

### Production Example 1

A mixture of 7-bromo-2,4-dichloro-8-fluoro-6-iodoquinazoline (100 g), DOX (1000 mL), and THF (500 mL) was cooled with ice bath, then DIPEA (240 mL) and tert-butyl (1S,4S)-2,5-diazabicyclo[2.2.1]heptane-2-carboxylate (48 g) were added, and the mixture was stirred at room temperature overnight. Water was added to the reaction mixture, and the mixture was extracted with ethyl acetate. The organic layer was washed with aqueous sodium chloride solution, dried over anhydrous magnesium sulfate, and then concentrated under reduced pressure until the total volume of the solution became about 400 mL. A mixed solvent (hexane/ethyl acetate=4/1, 1000 mL) was added to the resulting solution, and the mixture was stirred at room temperature. The precipitated solid was filtered to give tert-butyl (1S,4S)-5-(7-bromo-2-chloro-8-fluoro-6-iodoquinazolin-4-yl)-2,5-diazabicyclo[2.2.1]heptane-2-carboxylate (123 g) as a solid.

### Production Example 2

To a mixture of tert-butyl (1S,4S)-5-(7-bromo-2-chloro-8-fluoro-6-iodoquinazolin-4-yl)-2,5-diazabicyclo[2.2.1]heptane-2-carboxylate (30 g), tetrahydro-2H-pyran-4-ol (15.0 mL), DMF (150 mL), THF (100 mL), and DABCO (1.15 g) was added cesium carbonate (50.3 g) with stirring at room temperature, under an argon atmosphere, the mixture was stirred at room temperature overnight. About 1 kg of ice water was added to the reaction mixture, and the mixture was stirred at room temperature for 6 hours. The precipitated solid was filtered, washed with water, and dried under reduced pressure overnight to give tert-butyl (1S,4S)-5-{7-bromo-8-fluoro-6-iodo-2-[(oxan-4-yl)oxy]quinazolin-4-yl}-2,5-diazabicyclo[2.2.1]heptane-2-carboxylate (32.8 g) as a solid.

### Production Example 5

Under argon flow, to a mixture of tert-butyl (1S,4S)-5-{7-bromo-8-fluoro-6-iodo-2-[(oxan-4-yl)oxy]quinazolin-4-yl}-2,5-diazabicyclo[2.2.1]heptane-2-carboxylate (11.9 g), benzyl alcohol (2.37 g), and THF (40 mL) was added tBuOK (2.54 g) under ice-bath cooling, and the mixture was stirred at the same temperature for 1.5 hours. Ice water and saturated aqueous ammonium chloride solution were added to the reaction mixture, the mixture was extracted with ethyl acetate, and the organic layer was dried over anhydrous magnesium sulfate. The solution was concentrated under reduced pressure, and a mixed solvent of hexane/ethyl acetate (6/1) was added to the resulting residue. The mixture was stirred for a while, and the precipitated solid was filtered, and dried to give tert-butyl (1S,4S)-5-{8-(benzyloxy)-7-bromo-6-iodo-2-[(oxan-4-yl)oxy]quinazolin-4-yl}-2,5-diazabicyclo[2.2.1]heptane-2-carboxylate (11.8 g) as a solid.

### Production Example 8

Under an argon atmosphere, a mixture of tert-butyl (1S,4S)-5-{8-(benzyloxy)-7-bromo-6-iodo-2-[(oxan-4-yl)oxy]quinazolin-4-yl}-2,5-diazabicyclo[2.2.1]heptane-2-carboxylate (5.47 g), MeCN (88 mL), DOX (10 mL), water (22 mL), cyclopropylboronic acid (1.27 g), tripotassium phosphate (5.67 g), and PdCl₂ (dppf)·CH₂Cl₂ (600 mg) was stirred at 100° C. for 3 hours. After the reaction mixture was allowed to cool to room temperature, the solution was concentrated under reduced pressure. Saturated aqueous sodium chloride solution was added to the resulting residue, and the mixture was extracted with CHCl₃. The organic layer was dried over anhydrous magnesium sulfate, and the solution was concentrated under reduced pressure. The resulting residue was purified by silica gel column chromatography (hexane/ethyl acetate) to give tert-butyl (1S,4S)-5-(8-(benzyloxy)-7-bromo-6-cyclopropyl-2-[(oxan-4-yl)oxy]quinazolin-4-yl}-2,5-diazabicyclo[2.2.1]heptane-2-carboxylate (3.8 g) as a foam-like solid.

### Production Example 11

A mixture of tert-butyl (1S,4S)-5-(8-(benzyloxy)-7-bromo-6-cyclopropyl-2-[(oxan-4-yl)oxy]quinazolin-4-yl}-2,5-diazabicyclo[2.2.1]heptane-2-carboxylate (3.15 g), 6-fluoro-5-methyl-1-(tetrahydro-2H-pyran-2-yl)-4-(4,4,5,5-tetramethyl-1,3,2-dioxaborolan-2-yl)-1H-indazol (1.92 g), tripotassium phosphate (4.1 g), dicyclohexyl(2′,6′-diisopropoxy-[1, l′-biphenyl]-2-yl)phosphine (0.12 g), (2-dicyclohexylphosphino-2′,6′-diisopropoxy-1,1′-biphenyl) [2-(2′-amino-1,1′-biphenyl)]palladium(II) methanesulfonate (0.2 g), DOX (40 mL), and water (8 mL) was degassed and substituted with argon with stirring at room temperature, and then, the mixture was stirred at 100° C. for 2.5 hours under an argon atmosphere. Water (about 150 mL) was added to the reaction mixture which was cooled to room temperature, and the mixture was extracted with ethyl acetate. After the organic layer was dried over anhydrous magnesium sulfate, the insoluble materials were removed by filtration, and the filtrate was concentrated under reduced pressure. The resulting residue was purified by silica gel column chromatography (basic silica gel, hexane/ethyl acetate), and fractions respectively containing (1) low-polar diastereomeric mixture (peak-1 and peak-2; peak-1 and peak-2 had the same axial chirality) and (2) high-polar diastereomeric mixture (peak-3 and peak-4; peak-3 and peak-4 had the same axial chirality) were obtained. Of these fractions, the fractions containing the low-polar diastereomeric mixture (peak-1 and peak-2, the same axial chirality) were collected to give tert-butyl (1S,4S)-5-{8-(benzyloxy)-6-cyclopropyl-7-[6-fluoro-5-methyl-1-(oxan-2-yl)-1H-indazol-4-yl]-2-[(oxan-4-yl)oxy]quinazolin-4-yl}-2,5-diazabicyclo[2.2.1]heptane-2-carboxylate (1.42 g) as a foam-like solid. In addition, the fractions containing the high-polar diastereomeric mixture (peak-3 and peak-4, the same axial chirality) were collected to give tert-butyl (1S,4S)-5-{8-(benzyloxy)-6-cyclopropyl-7-[6-fluoro-5-methyl-1-(oxan-2-yl)-1H-indazol-4-yl]-2-[(oxan-4-yl)oxy]quinazolin-4-yl}-2,5-diazabicyclo[2.2.1]heptane-2-carboxylate (1.37 g) as a foam-like solid.

### Production Example 14

To a MeOH (200 mL) solution of tert-butyl (1S,4S)-5-{8-(benzyloxy)-6-cyclopropyl-7-[6-fluoro-5-methyl-1-(oxan-2-yl)-1H-indazol-4-yl]-2-[(oxan-4-yl)oxy]quinazolin-4-yl}-2,5-diazabicyclo[2.2.1]heptane-2-carboxylate (10 g) which was the low-polar diastereomeric mixture obtained in Production Example 11 was added 10% Pd/C (50% wet, 2 g), and the reaction mixture was stirred under hydrogen atmosphere at room temperature for 2 hours. The resulting reaction mixture was filtered through celite pad and washed with MeOH. The filtrate was concentrated under reduced pressure, and the resulting residue was purified by silica gel column chromatography (hexane/ethyl acetate) to give tert-butyl (1S,4S)-5-{6-cyclopropyl-7-[6-fluoro-5-methyl-1-(oxan-2-yl)-1H-indazol-4-yl]-8-hydroxy-2-[(oxan-4-yl)oxy]quinazolin-4-yl}-2,5-diazabicyclo[2.2.1]heptane-2-carboxylate (8.11 g) as a foam-like solid.

### Production Example 22

To a mixture of tert-butyl (1S,4S)-5-{6-cyclopropyl-7-[6-fluoro-5-methyl-1-(oxan-2-yl)-1H-indazol-4-yl]-8-hydroxy-2-[(oxan-4-yl)oxy]quinazolin-4-yl}-2,5-diazabicyclo[2.2.1]heptane-2-carboxylate (7.48 g), DMF (70 mL), and 1-(chloromethyl)-4-ethynylbenzene (1.9 g), was added cesium carbonate (6.2 g) with stirring at room temperature, and the mixture was stirred under an argon atmosphere at 60° C. for 2 hours. To the reaction mixture which was allowed to cool to room temperature, ice water and saturated aqueous ammonium chloride solution were added, and the mixture was extracted with ethyl acetate. The organic layer was washed with saturated aqueous sodium chloride solution, and dried Over anhydrous magnesium sulfate. Then, the insoluble materials were removed by filtration, and the filtrate was concentrated under reduced pressure. The resulting residue was purified by silica gel column chromatography (basic silica gel, hexane/ethyl acetate), and the resulting solid was filtered to give tert-butyl (1S,4S)-5-{6-cyclopropyl-8-[(4-ethynylphenyl)methoxy]-7-[6-fluoro-5-methyl-1-(oxan-2-yl)-1H-indazol-4-yl]-2-[(oxan-4-yl)oxy]quinazolin-4-yl}-2,5-diazabicyclo[2.2.1]heptane-2-carboxylate (8.12 g) as a foam-like solid.

### Production Example 32

To a CH₂Cl₂ (13 mL) solution of tert-butyl (1S,4S)-5-{8-(benzyloxy)-6-cyclopropyl-2-(dodecylsulfanyl)-7-[6-fluoro-5-methyl-1-(oxan-2-yl)-1H-indazol-4-yl]quinazolin-4-yl}-2,5-diazabicyclo[2.2.1]heptane-2-carboxylate (1.3 g) was added m-chloroperbenzoic acid (about 30% water content, 358 mg) under ice-bath cooling, and the mixture was stirred at the same temperature for 2 hours. Under ice-bath cooling, 10% aqueous sodium thiosulfate solution and saturated aqueous sodium hydrogen carbonate solution were added to the reaction mixture. The aqueous layer and the organic layer were separated, and the resulting aqueous layer was extracted with ethyl acetate. The combined organic layers were dried over anhydrous magnesium sulfate. The resulting solution was concentrated under reduced pressure to give tert-butyl (1S,4S)-5-{8-(benzyloxy)-6-cyclopropyl-2-(dodecane-1-sulfinyl)-7-[6-fluoro-5-methyl-1-(oxan-2-yl)-1H-indazol-4-yl]quinazolin-4-yl}-2,5-diazabicyclo[2.2.1]heptane-2-carboxylate (1.32 g) as an oil.

### Production Example 34

To a mixture of tert-butyl (1S,4S)-5-{8-(benzyloxy)-6-cyclopropyl-2-(dodecane-1-sulfinyl)-7-[6-fluoro-5-methyl-1-(oxan-2-yl)-1H-indazol-4-yl]quinazolin-4-yl}-2,5-diazabicyclo[2.2.1]heptane-2-carboxylate (1.32 g), DMAc (15 mL), and 4-ethyl-3-hydroxypyridine (525 mg) were added cesium carbonate (1.9 g) and DABCO (160 mg) at room temperature, and under nitrogen atmosphere, the mixture was stirred at 80° C. for 2 hours and at 100° C. for 2 hours. After the mixture was allowed to cool to room temperature, water was added to the reaction mixture, and the mixture was extracted with ethyl acetate. The organic layer was washed with water and saturated aqueous sodium chloride solution, and was then dried over anhydrous sodium sulfate, and the solution was concentrated under reduced pressure. The resulting residue was purified by silica gel column chromatography (hexane/ethyl acetate) to give tert-butyl (1S,4S)-5-{8-(benzyloxy)-6-cyclopropyl-2-[(4-ethylpyridin-3-yl)oxy]-7-[6-fluoro-5-methyl-1-(oxan-2-yl)-1H-indazol-4-yl]quinazolin-4-yl}-2,5-diazabicyclo[2.2.1]heptane-2-carboxylate (774 mg) as an oil.

### Production Example 40

To a mixture of tert-butyl (1S,4S)-5-{6-cyclopropyl-8-[(4-ethynylphenyl)methoxy]-7-[6-fluoro-5-methyl-1-(oxan-2-yl)-1H-indazol-4-yl]-2-[(oxan-4-yl)oxy]quinazolin-4-yl}-2,5-diazabicyclo[2.2.1]heptane-2-carboxylate (4.24 g), (4R)-1-[(2S)-2-azido-3-methylbutanoyl]-4-hydroxy-N-{(1R)-2-hydroxy-1-[4-(4-methyl-1,3-thiazol-5-yl)phenyl]ethyl}-L-prolinamide (2.30 g), sodium ascorbate (1.45 g), tert-butyl alcohol (35 mL), THF (35 mL), and water (35 mL), was added anhydrous copper (II) sulfate (389 mg) at room temperature, and the mixture was stirred at room temperature for 2.5 hours. Ethyl acetate and water were added, and the aqueous layer was separated. The aqueous layer was extracted with ethyl acetate, and the combined organic layer was washed with saturated aqueous sodium chloride solution, and dried over anhydrous sodium sulfate. The insoluble materials were removed by filtration, and the filtrate was concentrated under reduced pressure. The residue was purified by silica gel column chromatography (CHCl₃/MeOH) to give tert-butyl (1S,4S)-5-{6-cyclopropyl-7-[6-fluoro-5-methyl-1-(oxan-2-yl)-1H-indazol-4-yl]-8-{[4-(1-{(2)-1-[(2S,4R)-4-hydroxy-2-{(1R)-2-hydroxy-1-[4-(4-methyl-1,3-thiazol-5-yl)phenyl]ethyl}carbamoyl) pyrrolidin-1-yl]-3-methyl-1-oxobutan-2-yl}-1H-1,2,3-triazol-4-yl)phenyl]methoxy}-2-[(oxan-4-yl)oxy]quinazolin-4-yl}-2,5-diazabicyclo[2.2.1]heptane-2-carboxylate (5.62 g) as a solid.

### Production Example 51

Under an argon atmosphere, to a mixture of tert-butyl (1S,4S)-5-{6-cyclopropyl-7-[6-fluoro-5-methyl-1-(oxan-2-yl)-1H-indazol-4-yl]-8-{[4-(1-{(2S)-1-[(2S,4R)-4-hydroxy-2-({(1R)-2-hydroxy-1-[4-(4-methyl-1,3-thiazol-5-yl)phenyl]ethyl}carbamoyl) pyrrolidin-1-yl]-3-methyl-1-oxobutan-2-yl}-1H-1,2,3-triazol-4-yl)phenyl]methoxy}-2-[(1-{[2-(trimethylsilyl)ethoxy]carbonyl}piperidin-4-yl)oxy]quinazolin-4-yl}-2,5-diazabicyclo[2.2.1]heptane-2-carboxylate (1.1 g), THF (22 mL), and tetra-n-butylammonium fluoride (1M THF solution, 2.57 mL) was added acetic acid (90 μL) at room temperature, and the mixture was stirred at 60° C. for 15 hours. After the mixture was allowed to cool to room temperature, ethyl acetate and saturated aqueous ammonium chloride solution were added, and the mixture was separated into layers. The aqueous layer was extracted with ethyl acetate/methanol (10/1), and the combined organic layer was washed with saturated aqueous sodium chloride solution, and dried over anhydrous sodium sulfate. The insoluble materials were removed by filtration, and the filtrate was concentrated under reduced pressure. The residue was purified by silica gel column chromatography (basic silica gel, CHCl₃/MeOH) to give tert-butyl (1S,4S)-5-{6-cyclopropyl-7-[6-fluoro-5-methyl-1-(oxan-2-yl)-1H-indazol-4-yl]-8-{[4-(1-{(2S)-1-[(2S,4R)-4-hydroxy-2-{(1R)-2-hydroxy-1-[4-(4-methyl-1,3-thiazol-5-yl)phenyl]ethyl}carbamoyl) pyrrolidin-1-yl]-3-methyl-1-oxobutan-2-yl}-1H-1,2,3-triazol-4-yl)phenyl]methoxy}-2-[(piperidin-4-yl)oxy]quinazolin-4-yl}-2,5-diazabicyclo[2.2.1]heptane-2-carboxylate (951 mg) as a solid.

### Production Example 52

To a mixture of tert-butyl (1S,4S)-5-{6-cyclopropyl-7-[6-fluoro-5-methyl-1-(oxan-2-yl)-1H-indazol-4-yl]-8-{[4-(1-{(2S)-1-[(2S,4R)-4-hydroxy-2-{(1R)-2-hydroxy-1-[4-(4-methy]-1,3-thiazol-5-yl)phenyl]ethyl}carbamoyl) pyrrolidin-1-yl]-3-methyl-1-oxobutan-2-yl}-1H-1,2,3-triazol-4-yl)phenyl]methoxy}-2-[(piperidin-4-yl)oxy]quinazolin-4-yl}-2,5-diazabicyclo[2.2.1]heptane-2-carboxylate (250 mg), oxetan-3-One (43 mg), and CH₂Cl₂ (3 mL) was added sodium triacetoxyborohydride (122 mg) at room temperature, and the mixture was stirred at room temperature for 16 hours. Saturated aqueous sodium hydrogen carbonate solution was added, and the mixture was stirred at room temperature for 10 minutes. The mixture was extracted with CHCl₃/MeOH (5/1), and the combined organic layer was dried over anhydrous sodium sulfate. The insoluble materials were removed by filtration, and the filtrate was concentrated under reduced pressure. The residue was purified by silica gel column chromatography (CHCl₃/MeOH) to give tert-butyl (1S,4S)-5-(6-cyclopropyl-7-[6-fluoro-5-methyl-1-(oxan-2-yl)-1H-indazol-4-yl]-8-{[4-(1-{(2S)-1-{(2S,4R)-4-hydroxy-2-({(1R)-2-hydroxy-1-[4-(4-methyl-1,3-thiazol-5-yl)phenyl]ethyl}carbamoyl) pyrrolidin-1-yl]-3-methyl-1-oxobutan-2-yl}-1H-1,2,3-triazol-4-yl)phenyl]methoxy}-2-{[1-(oxetan-3-yl) piperidin-4-yl]oxy}quinazolin-4-yl)-2,5-diazabicyclo[2.2.1]heptane-2-carboxylate (220 mg) as a solid.

### Production Example 53

To a mixture of tert-butyl (1S,4S)-5-{6-cyclopropyl-7-[6-fluoro-5-methyl-1-(oxan-2-yl)-1H-indazol-4-yl]-8-{[4-(1-{(2S)-1-[(2S,4R)-4-hydroxy-2-({(1R)-2-hydroxy-1-[4-(4-methyl-1,3-thiazol-5-yl)phenyl]ethyl}carbamoyl) pyrrolidin-1-yl]-3-methyl-1-oxobutan-2-yl}-1H-1,2,3-triazol-4-yl)phenyl]methoxy}-2-[(piperidin-4-yl)oxy]quinazolin-4-yl}-2,5-diazabicyclo[2.2.1]heptane-2-carboxylate (250 mg), 2,2-difluoroethyl trifluoromethanesulfonate (124 mg), and MeCN (3 mL) was added DIPEA (99 μL) at room temperature, and the mixture was stirred at room temperature for 16 hours. The mixture was concentrated under reduced pressure, and the residue was purified by silica gel column chromatography (CHCl₃/MeOH) to give tert-butyl (1S,4S)-5-(6-cyclopropyl-2-{[1-(2,2-difluoroethyl) piperidin-4-yl]oxy}-7-[6-fluoro-5-methyl-1-(oxan-2-yl)-1H-indazol-4-yl]-8-{[4-(1-{(2S)-1-[(2S,4R)-4-hydroxy-2-({(1R)-2-hydroxy-1-[4-(4-methyl-1,3-thiazol-5-yl)phenyl]ethyl}carbamoyl) pyrrolidin-1-yl]-3-methyl-1-oxobutan-2-yl}-1H-1,2,3-triazol-4-yl)phenyl]methoxy}quinazolin-4-yl)-2,5-diazabicyclo[2.2.1]heptane-2-carboxylate (196 mg) as a solid.

### Production Example 54

To a CH₂Cl₂ (12 mL) solution of methyl (2S)-2-[5-(hydroxymethyl)-1-oxo-1,3-dihydro-2H-isoindol-2-yl]-3-methylbutanoate (190 mg) was added thionyl chloride (500 μL) under an argon atmosphere under ice-bath cooling, and the mixture was stirred at the same temperature for 2 hours. The reaction mixture was concentrated under reduced pressure, and to the resulting residue, DMF (7 mL), tert-butyl (1S,4S)-5-{6-cyclopropyl-7-[6-fluoro-5-methyl-1-(oxan-2-yl)-1H-indazol-4-yl]8-hydroxy-2-[(oxan-4-yl)oxy]quinazolin-4-yl}-2,5-diazabicyclo[2.2.1]heptane-2-carboxylate (450 mg), and cesium carbonate (260 mg) were added under ice-bath cooling. The mixture was stirred at 60° C. overnight under an argon atmosphere. The reaction solution was filtered through celite pad, and the residue on the celite was washed with ethyl acetate. The filtrate was concentrated under reduced pressure, and the resulting residue was purified by silica gel column chromatography (basic silica gel, hexane/ethyl acetate) to give tert-butyl (1S,4S)-5-{6-cyclopropyl-7-[6-fluoro-5-methyl-1-(oxan-2-yl)-1H-indazol-4-yl]-8-{2-[(2)-1-methoxy-3-methyl-1-oxobutan-2-yl]-1-oxo-2,3-dihydro-1H-isoindol-5-yl}methoxy)-2-[(oxan-4-yl)oxy]quinazolin-4-yl}-2,5-diazabicyclo[2.2.1]heptane-2-carboxylate (670 mg) as a foam-like solid.

### Production Example 56

To a MeOH (7 mL) solution of tert-butyl (1S,4S)-5-{6-cyclopropyl-7-[6-fluoro-5-methyl-1-(oxan-2-yl)-1H-indazol-4-yl]8-({2-[(2S)-1-methoxy-3-methyl-1-oxobutan-2-yl]-1-oxo-2,3-dihydro-1H-isoindol-5-yl}methoxy)-2-[(oxan-4-yl)oxy]quinazolin-4-yl}-2,5-diazabicyclo[2.2.1]heptane-2-carboxylate (670 mg) was added an aqueous sodium hydroxide solution (1M, 2.5 mL) under ice-bath cooling, and the mixture was stirred for 3 days at room temperature. Under ice-bath cooling, hydrochloric acid (1M, 2.5 mL) was added to neutralize the mixture. Then, CHCl₃ and water were added to divide the mixture into layers, and the aqueous layer was extracted with CHCl₃. The combined organic layer was dried over anhydrous sodium sulfate, then the insoluble materials were removed by filtration, and the filtrate was concentrated under reduced pressure. The resulting residue was purified by silica gel column chromatography (CHCl₃/MeOH), and then was purified again by silica gel column chromatography (basic silica gel, CHCl₃/MeOH) to give (2S)-2-{5-[({4-{(1S,4S)-5-(tert-butoxycarbonyl)-2,5-diazabicyclo[2.2.1]heptan-2-yl]-6-cyclopropyl-7-[6-fluoro-5-methyl-1-(oxan-2-yl)-1H-indazol-4-yl]2-[(oxan-4-yl)oxy]quinazolin-8-yl}oxy)methyl]-1-oxo-1,3-dihydro-2H-isoindol-2-yl}-3-methylbutanoic acid (372 mg) as a foam-like solid.

### Production Example 61

To a 1,2-dichloroethane (60 mL) solution of tert-butyl (1S,4S)-5-{6-cyclopropyl-7-[6-fluoro-5-methyl-1-(oxan-2-yl)-1H-indazol-4-yl]-8-{[4-(1-{(2S)-1-[(2S,4R)-4-hydroxy-2-(methoxycarbonyl) pyrrolidin-1-yl]-3-methyl-1-oxobutan-2-yl}-1H-1,2,3-triazol-4-yl)phenyl]methoxy}-2-[(oxan-4-yl)oxy]quinazolin-4-yl}-2,5-diazabicyclo[2.2.1]heptane-2-carboxylate (3.97 g) was added trimethyltin (IV) hydroxide (3.35 g) at room temperature, and the mixture was stirred at 80° C. for 18 hours. After the mixture was allowed to cool to room temperature, hydrochloric acid (1M, 60 mL) was added, and the mixture was extracted with CHCl₃/MeOH (9/1). The organic layer was washed with 1M hydrochloric acid, and dried over anhydrous sodium sulfate. The insoluble materials were removed by filtration, and the filtrate was concentrated under reduced pressure. The residue was purified by silica gel column chromatography (CHCl₃/MeOH) to give (4R)-1-[(2S)-2-(4-{4-[({4-[(1S,4S)-5-(tert-butoxycarbonyl)-2,5-diazabicyclo[2.2.1]heptan-2-yl]-6-cyclopropyl-7-[6-fluoro-5-methyl-1-(oxan-2-yl)-1H-indazol-4-yl]-2-{(oxan-4-yl)oxy]quinazolin-8-yl}oxy)methyl]phenyl}-1H-1,2,3-triazol-1-yl)-3-methylbutanoyl]-4-hydroxy-L-proline (3.26 g) as a solid.

### Production Example 63

To a mixture of (4R)-1-[(2S)-2-(4-{4-[({4-[(1S,4S)-5-(tert-butoxycarbonyl)-2,5-diazabicyclo[2.2.1]heptan-2-yl]-6-cyclopropyl-7-[6-fluoro-5-methyl-1-(oxan-2-yl)-1H-indazol-4-yl]-2-[(oxan-4-yl)oxy]quinazolin-8-yl}oxy)methyl]phenyl}-1H-1,2,3-triazol-1-yl)-3-methylbutanoyl]-4-hydroxy-L-proline (150 mg), 3-{4-[(1R)-1-amino-2-hydroxyethyl]phenyl}-1,3-oxazolidin-2-one n-hydrochloride (60 mg), DIPEA (70 μL), and DMF (3 mL) was added HATU (70 mg) under ice-bath cooling, and the mixture was stirred under ice-bath cooling for 1 hour. Water, saturated aqueous sodium chloride solution, and ethyl acetate were added to the mixture, and the aqueous layer was separated. The aqueous layer was extracted with ethyl acetate, and the combined organic layer was washed with water and saturated aqueous sodium chloride solution, and dried over anhydrous magnesium sulfate. The insoluble materials were removed by filtration, and the filtrate was concentrated under reduced pressure. The residue was purified by silica gel column chromatography (CHCl₃/MeOH) to give tert-butyl (1S,4S)-5-{6-cyclopropyl-7-[6-fluoro-5-methyl-1-(oxan-2-yl)-1H-indazol-4-yl]-8-{[4-(1-{(2S)-1-[(2S,4R)-4-hydroxy-2-({(1R)-2-hydroxy-1-[4-(2-oxo-1,3-oxazolidin-3-yl)phenyl]ethyl}carbamoyl) pyrrolidin-1-yl]-3-methyl-1-oxobutan-2-yl}-1H-1,2,3-triazol-4-yl)phenyl]methoxy}-2-[(oxan-4-yl)oxy]quinazolin-4-yl}-2,5-diazabicyclo[2.2.1]heptane-2-carboxylate (173 mg) as a solid.

### Production Example 66

A mixture of tert-butyl (1S,4S)-5-{8-(benzyloxy)-7-bromo-6-cyclopropyl-2-[(oxan-4-yl)oxy]quinazolin-4-yl}-2,5-diazabicyclo[2.2.1]heptane-2-carboxylate (6.5 g), 4,4,4′,4′,5,5,5′,5′-octamethyl-2,2′-bi-1,3,2-dioxaborolane (7.6 g), triphenylphosphine (0.53 g), potassium acetate (4.9 g), DOX (120 mL), and palladium acetate (0.23 g) was degassed and substituted with argon gas, and was stirred at 115° C. overnight. The reaction solution which was allowed to cool to room temperature was filtered through celite pad while washing the celite pad with a small amount of dioxane, and the filtrate was concentrated under reduced pressure. The residue was purified by silica gel column chromatography (hexane/ethyl acetate) to give tert-butyl (1S,4S)-5-[8-(benzyloxy)-6-cyclopropyl-2-[(oxan-4-yl)oxy]-7-(4,4,5,5-tetramethyl-1,3,2-dioxaborolan-2-yl)quinazolin-4-yl]-2,5-diazabicyclo[2.2.1]heptane-2-carboxylate (5.47 g) as a foam-like solid.

### Production Example 67

Tert-butyl (1S,4S)-5-[8-(benzyloxy)-6-cyclopropyl-2-[(oxan-4-yl)oxy]-7-(4,4,5,5-tetramethyl-1,3,2-dioxaborolan-2-yl)quinazolin-4-yl]-2,5-diazabicyclo[2.2.1]heptane-2-carboxylate (1.52 g), (3-bromo-5-fluoro-4-methylphenoxy) (tert-butyl)di(methyl) silane (0.84 g), tripotassium phosphate (1.85 g), dicyclohexyl(2′,6′-diisopropoxy-[1,1′-biphenyl]-2-yl)phosphine (0.15 g), (2-dicyclohexylphosphino-2′,6′-diisopropoxy-1,1′-biphenyl) [2-(2′-amino-1,1′-biphenyl)]palladium(II) methanesulfonate (0.27 g), DOX (20 mL), and water (4 mL) were sequentially added to flask, and while stirring the mixture at room temperature, degassing/argon gas substitution operation was performed. Then, the mixture was stirred under an argon atmosphere at 90° C. for 5 hours. The mixture was further stirred at 100° C. for 7 hours. The reaction solution which was allowed to cool to room temperature was diluted with water and extracted with ethyl acetate. The organic layer was dried over anhydrous magnesium sulfate, and the solution was concentrated under reduced pressure. The resulting residue was purified by silica gel column chromatography (basic silica gel, hexane/ethyl acetate) to give fractions respectively containing (1) a low-polar diastereomer (peak-1) and (2) a high-polar diastereomer (peak-2). Of these fractions, the fractions containing the low-polar diastereomer (peak-1) were collected to give tert-butyl (1S,4S)-5-{8-(benzyloxy)-6-cyclopropyl-7-(3-fluoro-5-hydroxy-2-methylphenyl)-2-[(oxan-4-yl)oxy]quinazolin-4-yl}-2,5-diazabicyclo[2.2.1]heptane-2-carboxylate (580 mg) as a foam-like solid. In addition, the fractions containing the high-polar diastereomer (peak-2) were collected to give tert-butyl (1S,4S)-5-{8-(benzyloxy)-6-cyclopropyl-7-(3-fluoro-5-hydroxy-2-methylphenyl)-2-[(oxan-4-yl)oxy]quinazolin-4-yl}-2,5-diazabicyclo[2.2.1]heptane-2-carboxylate (360 mg) as a foam-like solid.

### Production Example 68

Under nitrogen atmosphere, to a DMF (5 mL) solution of tert-butyl (1S,4S)-5-{8-(benzyloxy)-6-cyclopropyl-7-(3-fluoro-5-hydroxy-2-methylphenyl)-2-[(oxan-4-yl)oxy]quinazolin-4-yl}-2,5-diazabicyclo[2.2.1]heptane-2-carboxylate (250 mg) were added cesium carbonate (180 mg) and chloro(methoxy)methane (35 μL) under ice-bath cooling, and the mixture was stirred at room temperature for 15 hours. Under ice-bath cooling, cesium carbonate (260 mg) and chloro(methoxy)methane (50 μL) were added, and the mixture was further stirred at room temperature for 2 hours. The reaction mixture was diluted with ethyl acetate, and washed with water and saturated aqueous sodium chloride solution. After the organic layer was dried over anhydrous magnesium sulfate, the insoluble materials were removed by filtration, and the filtrate was concentrated under reduced pressure.

The resulting residue was purified by silica gel column chromatography (hexane/ethyl acetate) to give tert-butyl (1S,4S)-5-{8-(benzyloxy)-6-cyclopropyl-7-[3-fluoro-5-(methoxymethoxy)-2-methylphenyl]-2-[(oxan-4-yl)oxy]quinazolin-4-yl}-2,5-diazabicyclo[2.2.1]heptane-2-carboxylate (186 mg) as an oil.

### Production Example 69

Under an argon atmosphere, to a mixture of tert-butyl N-[(1R)-1-(4-bromophenyl)-2-hydroxyethyl]carbamate (4.43 g), 1-ethyl-5-(4,4,5,5-tetramethyl-1,3,2-dioxaborolan-2-yl)-1H-pyrazole (4.67 g), potassium carbonate (3.87 g), DOX (80 mL), and water (8 mL) was added PdCl₂ (dppf)·CH₂Cl₂ (1.14 g), and the mixture was stirred at 100° C. for 16 hours. After the mixture was allowed to cool to room temperature, ethyl acetate was added, and the mixture was filtered through celite pad and concentrated under reduced pressure. The residue was purified by silica gel column chromatography (basic silica gel, hexane/ethyl acetate) to give tert-butyl {(1R)-1-[4-(1-ethyl-1H-pyrazol-5-yl)phenyl]-2-hydroxyethyl}carbamate (3.74 g) as a solid.

### Production Example 71

To a solution in CH₂Cl₂ (25 mL) and MeOH (25 mL) of tert-butyl {(1R)-1-[4-(1-ethyl-1H-pyrazol-5-yl)phenyl]-2-hydroxyethyl}carbamate (3.34 g), hydrogen chloride (4M DOX solution, 25.6 mL) was added at −20 to −10° C., and the mixture was stirred at room temperature for 5 hours. The reaction mixture was concentrated under reduced pressure to give (2R)-2-amino-2-[4-(1-ethyl-1H-pyrazol-5-yl)phenyl]ethan-1-ol n-hydrochloride (3.06 g) as a solid.

### Production Example 73

To a mixture of (2R)-2-amino-2-[4-(1-ethyl-1H-pyrazol-5-yl)phenyl]ethan-1-ol n-hydrochloride (3.43 g), (4R)-1-(tert-butoxycarbonyl)-4-hydroxy-L-proline (2.81 g), and DMF (40 mL) was added DIPEA (7.8 mL) under ice-bath cooling, and then HATU (4.5 g) was added portionwise under ice-bath cooling. The mixture was stirred for 1 hour under ice-bath cooling and 1 hour at room temperature. Under ice-bath cooling, water, saturated aqueous sodium chloride solution, and ethyl acetate were added, and the aqueous layer was extracted with ethyl acetate, and then extracted with ethyl acetate/isopropyl alcohol (9/1). The combined organic layer was washed with saturated aqueous sodium chloride solution, and dried over anhydrous magnesium sulfate. The insoluble materials were removed by filtration, and the filtrate was concentrated under reduced pressure. The residue was purified by silica gel column chromatography (CHCl₃/MeOH) to give tert-butyl (2S,4R)-2-({(1R)-1-[4-(1-ethyl-1H-pyrazol-5-yl)phenyl]-2-hydroxyethyl}carbamoyl)-4-hydroxypyrrolidine-1-carboxylate (5.01 g) as an oil.

### Production Example 76

To a solution in CH₂Cl₂ (35 mL) and MeOH (30 mL) of tert-butyl (2S,4R)-2-({(1R)-1-[4-(1-ethyl-1H-pyrazol-5-yl)phenyl]-2-hydroxyethyl}carbamoyl)-4-hydroxypyrrolidine-1-carboxylate (5.01 g) was added hydrogen chloride (4M DOX solution, 28 mL) at −20 to −10° C., and the mixture was stirred at room temperature for 5 hours. The reaction mixture was concentrated under reduced pressure to give (4R)—N-{(1R)-1-[4-(1-ethyl-1H-pyrazol-5-yl)phenyl]-2-hydroxyethyl}-4-hydroxy-L-prolinamide n-hydrochloride (4.71 g) as a solid.

### Production Example 79

To a mixture of (4R)-4-hydroxy-N-{(1R)-2-hydroxy-1-[4-(4-methyl-1,3-thiazol-5-yl)phenyl]ethyl}-L-prolinamide n-hydrochloride (3.81 g), N-(tert-butoxycarbonyl)-L-valine (2.16 g), and DMF (45 mL) was added DIPEA (6.2 mL), then HATU (3.61 g) was added portionwise under ice-bath cooling. The mixture was stirred for 1 hour under ice-bath cooling and for 1 hour at room temperature. Under ice-bath cooling, water, saturated aqueous sodium chloride solution, and ethyl acetate were added, and the aqueous layer was extracted with ethyl acetate, then extracted with ethyl acetate/isopropyl alcohol (9/1). The combined organic layer was washed with saturated aqueous sodium chloride solution, and dried over anhydrous sodium sulfate. The insoluble materials were removed by filtration, and the filtrate was concentrated under reduced pressure. The residue was purified by silica gel column chromatography (CHCl₃/MeOH) to give N-(tert-butoxycarbonyl)-L-valyl-(4R)-4-hydroxy-N-{(1R)-2-hydroxy-1-[4-(4-methyl-1,3-thiazol-5-yl)phenyl]ethyl}-L-prolinamide (4.43 g) as a solid.

### Production Example 83

To a solution in CH₂Cl₂ (35 mL) and MeOH (35 mL) of N-(tert-butoxycarbonyl)-L-valyl-(4R)-4-hydroxy-N-{(1R)-2-hydroxy-1-[4-(4-methyl-1,3-thiazol-5-yl)phenyl]ethyl}-L-prolinamide (4.43 g) was added hydrogen chloride (4M DOX solution, 20 mL) at −20 to −15° C., and the mixture was stirred at room temperature for 6 hours. The reaction mixture was concentrated under reduced pressure to give L-valyl-(4R)-4-hydroxy-N-{(1R)-2-hydroxy-1-[4-(4-methyl-1,3-thiazol-5-yl)phenyl]ethyl}-L-prolinamide n-hydrochloride (4.21 g) as a solid.

### Production Example 88

To a mixture of L-valyl-(4R)-4-hydroxy-N-{(1R)-2-hydroxy-1-[4-(4-methyl-1,3-thiazol-5-yl)phenyl]ethyl}-L-prolinamide n-hydrochloride (1.71 g), TEA (3.2 mL), THF (20 mL), and MeCN (20 mL) was added a MeCN (5 mL) solution of 2-azido-1,3-dimethylimidazolinium hexafluorophosphate (1.06 g) dropwise over 10 minutes or more under ice-bath cooling, and the mixture was stirred under ice-bath cooling for 5 hours. Water, saturated aqueous sodium chloride solution, and ethyl acetate were added, and the aqueous layer was separated. The aqueous layer was extracted with ethyl acetate, and the combined organic layer was dried over anhydrous sodium sulfate. The insoluble materials were removed by filtration, and the filtrate was concentrated under reduced pressure. The residue was purified by silica gel column chromatography (CHCl₃/MeOH) to give (4R)-1-[(2S)-2-azido-3-methylbutanoyl]-4-hydroxy-N-{(1R)-2-hydroxy-1-[4-(4-methyl-1,3-thiazol-5-yl)phenyl]ethyl}-L-prolinamide (1.07 g) as a solid.

### Production Example 93

To a mixture of L-valine methyl ester hydrochloride (1.96 g), MeCN (45 mL), and DIPEA (5 mL) was added methyl 4-bromo-2-(bromomethyl)benzoate (3.00 g) under water-bath cooling, and the mixture was slowly warmed up to 80° C., and stirred at 80° C. for 2 days. After the mixture was allowed to cool to room temperature, ethyl acetate and water were added, and the mixture was extracted with ethyl acetate. The combined organic layer was washed with saturated aqueous sodium chloride solution, and was dried over anhydrous sodium sulfate. The insoluble materials were removed by filtration, and the filtrate was concentrated under reduced pressure. The residue was purified by silica gel column chromatography (CHCl₃/ethyl acetate) to give methyl (2S)-2-(5-bromo-1-oxo-1,3-dihydro-2H-isoindol-2-yl)-3-methylbutanoate (2.86 g) as a solid.

### Production Example 94

Under an argon atmosphere, to a mixture of methyl (2S)-2-(5-bromo-1-oxo-1,3-dihydro-2H-isoindol-2-yl)-3-methylbutanoate (600 mg), potassium (2-trimethylsilyl)-ethoxymethyltrifluoroborate (876 mg), dicyclohexyl(2′,6′-dimethoxybiphenyl-2-yl)phosphine (151 mg), sodium carbonate (390 mg), DOX (9 mL), and water (1.8 mL) was added palladium (II) acetate (41 mg) at room temperature, and the mixture was stirred at 130° C. under microwave irradiation for 4 hours. After the mixture was allowed to cool to room temperature, ethyl acetate was added, and then the mixture was filtered through celite pad and washed with ethyl acetate. Water was added to the obtained filtrate to divide the mixture into layers, and the organic layer was washed with saturated aqueous sodium chloride solution. After the organic layer was dried over anhydrous sodium sulfate, the insoluble materials were removed by filtration, and the filtrate was concentrated under reduced pressure. The resulting residue was purified by silica gel column chromatography (basic silica gel, hexane/ethyl acetate) to give a coupling reaction product (600 mg). To a CH₂Cl₂ (4.2 mL) solution of the obtained coupling reaction product was added trifluoroacetic acid (2.1 mL) under ice-bath cooling, and the mixture was stirred at room temperature for 2 hours. The mixture was concentrated under reduced pressure, and the resulting residue was purified by silica gel column chromatography (basic silica gel, hexane/ethyl acetate) to give methyl (2S)-2-[5-(hydroxymethyl)-1-oxo-1,3-dihydro-2H-isoindol-2-yl]-3-methylbutanoate (190 mg) as a solid.

### Production Example 95

Under an argon atmosphere, to a mixture of ethyl 3-methyl-2-[4-(4,4,5,5-tetramethyl-1,3,2-dioxaborolan-2-yl)-1H-pyrazol-1-yl]butanoate (190 mg), 4-bromobenzyl alcohol (100 mg), PdCl₂ (dppf) CH₂Cl₂ (45 mg), and tripotassium phosphate (227 mg) were added DOX (2 mL) and water (0.4 mL), and the mixture was stirred at 100° C. for 12 hours. The reaction mixture was cooled to room temperature and was then filtered through celite pad, and the celite pad was washed with ethyl acetate. The filtrate was diluted with ethyl acetate, washed with water and saturated aqueous sodium chloride solution, and dried over anhydrous magnesium sulfate. The insoluble materials were removed by filtration, and the filtrate was concentrated under reduced pressure. The resulting residue was purified by silica gel column chromatography (hexane/ethyl acetate) to give ethyl 2-{4-[4-(hydroxymethyl)phenyl]-1H-pyrazol-1-yl}-3-methylbutanoate (157 mg) as an oily substance.

### Production Example 96

Under an argon atmosphere, to a THF (80 mL) solution of 2,2,6,6-tetramethylpyperidine (4.4 mL) was added n-butyllithium (1.57 M hexane solution, 15.2 mL) dropwise under cooling with a dry ice-MeOH bath (−78° C.), and the mixture was stirred under ice-bath cooling for 1 hour (light yellow solution). While cooling the reaction mixture with a dry ice-MeOH bath, a THF (20 mL) solution of (3-bromo-5-fluorophenoxy) (tert-butyl)di(methyl) silane (5.21 g) was added, and the mixture was stirred at the same temperature for 1 hour. Methyl iodide (2.2 mL) was added dropwise to the reaction mixture, and the mixture was stirred at the same temperature for 1 hour. To the reaction mixture, saturated aqueous ammonium chloride solution was added, and the mixture was stirred while increasing the temperature to room temperature. Ethyl acetate was added, and the reaction mixture was extracted with ethyl acetate, and the organic layer was washed with saturated aqueous sodium chloride solution, and dried over anhydrous sodium sulfate. The insoluble materials were removed by filtration, and the filtrate was concentrated under reduced pressure. The resulting residue was purified by silica gel column chromatography (hexane/ethyl acetate) to give (3-bromo-5-fluoro-4-methylphenoxy) (tert-butyl)di(methyl) silane (5.13 g) as an oil.

### Production Example 97

To a mixture of tert-butyl N-[(1R)-1-(4-bromophenyl)-2-hydroxyethyl]carbamate (500 mg), N-methyl-2-nitrobenzene sulfonamide (376 mg), tri-n-butylphosphine (0.51 mL), and THF (7 mL) was added 1,1′-azobis(N, N-dimethylformamide) (353 mg) portionwise under ice-bath cooling, and the mixture was stirred at room temperature for 8 hours. The mixture was diluted with ethyl acetate, washed with saturated aqueous sodium hydrogen carbonate solution, water, and saturated aqueous sodium chloride solution, and then dried over anhydrous magnesium sulfate. The insoluble materials were removed by filtration, and the filtrate was concentrated under reduced pressure. The residue was purified by silica gel chromatography (hexane/ethyl acetate) to give tert-butyl {(1R)-1-(4-bromophenyl)-2-[methyl (2-nitrobenzene-1-sulfonyl)amino]ethyl}carbamate (667 mg) as a solid.

### Production Example 98

Under an argon atmosphere, to a mixture of tert-butyl {(1R)-1-(4-bromophenyl)-2-[methyl (2-nitrobenzene-1-sulfonyl)amino]ethyl}carbamate (665 mg), 4-methyl-1,3-thiazole (235 μL), potassium acetate (253 mg), and DMAc (13 mL) was added palladium (II) acetate (29 mg), and the mixture was stirred at 100° C. for 16 hours. After the mixture was allowed to cool to room temperature, ethyl acetate and water were added, and the insoluble materials were filtered through celite pad. The filtrate was divided into layers, the aqueous layer was extracted with ethyl acetate, and the combined organic layer was washed with water and saturated aqueous sodium chloride solution, and dried over anhydrous magnesium sulfate. The insoluble materials were removed by filtration, and the filtrate was concentrated under reduced pressure. The residue was purified by silica gel column chromatography (hexane/ethyl acetate) to give tert-butyl {(1R)-2-[methyl (2-nitrobenzene-1-sulfonyl)amino]-1-[4-(4-methyl-1,3-thiazol-5-yl)phenyl]ethyl}carbamate (268 mg) as a solid.

### Production Example 99

Under an argon atmosphere, to a mixture of tert-butyl {(1R)-2-[methyl (2-nitrobenzene-1-sulfonyl)amino]-1-[4-(4-methyl-1,3-thiazol-5-yl)phenyl]ethyl}carbamate (130 mg), potassium carbonate (84 mg), and DMF (1.3 mL) was added 4-tert-butylbenzenethiol (82 μL) at room temperature, and the mixture was stirred at room temperature for 3 hours. Ethyl acetate and water were added, and the aqueous layer was extracted with ethyl acetate, and the combined organic layer was washed with water and saturated aqueous sodium chloride solution, and dried over anhydrous sodium sulfate. The insoluble materials were removed by filtration, and the filtrate was concentrated under reduced pressure. The residue was purified by silica gel column chromatography (CHCl₃/MeOH) to give tert-butyl {(1R)-2-(methylamino)-1-[4-(4-methyl-1,3-thiazol-5-yl)phenyl]ethyl}carbamate (60 mg) as an oil.

### Production Example 100

To a mixture of tert-butyl {(1R)-2-(methylamino)-1-[4-(4-methyl-1,3-thiazol-5-yl)phenyl]ethyl}carbamate (55 mg) and THF (1 mL) was added formaldehyde (37% aqueous solution, 26 μL) under ice-bath cooling, and the mixture was stirred under the same conditions for 10 minutes. Sodium triacetoxyborohydride (67 mg) was added thereto, and the mixture was stirred at room temperature for 1 hour. CHCl₃ was added to dilute the mixture, then saturated aqueous sodium hydrogen carbonate solution was added. The mixture was stirred for a while, and the aqueous layer was extracted with CHCl₃/MeOH (5/1), and the combined organic layer was dried over anhydrous sodium sulfate. The insoluble materials were removed by filtration, and the filtrate was concentrated under reduced pressure to give tert-butyl {(1R)-2-(dimethylamino)-1-[4-(4-methyl-1,3-thiazol-5-yl)phenyl]ethyl}carbamate (75 mg) as an oil.

### Production Example 102

A mixture of tert-butyl N-[(1R)-1-(4-bromophenyl)-2-hydroxyethyl]carbamate (2.04 g), 4,4,4′,4′,5,5,5′,5′-octamethyl-2,2′-bi-1,3,2-dioxaborolane (2.05 g), potassium acetate (1.91 g), DOX (40 mL), and bis(triphenylphosphine)palladium(II) dichloride (460 mg) was stirred under an argon atmosphere at 100° C. overnight. The reaction solution which was allowed to cool to room temperature was diluted with ethyl acetate, and the mixture was filtered through celite pad. The filtrate was washed with water and saturated aqueous sodium chloride solution, and dried over anhydrous magnesium sulfate. The insoluble materials were removed by filtration, and the filtrate was concentrated under reduced pressure. The residue was purified by silica gel column chromatography (hexane/ethyl acetate) to give tert-butyl {(1R)-2-hydroxy-1-[4-(4,4,5,5-tetramethyl-1,3,2-dioxaborolan-2-yl)phenyl]ethyl}carbamate (3.21 g) as an oil.

### Production Example 103

Under an argon atmosphere, to a mixture of tert-butyl {(1R)-2-hydroxy-1-[4-(4,4,5,5-tetramethyl-1,3,2-dioxaborolan-2-yl)phenyl]ethyl}carbamate (3.21 g), 5-bromo-1,3-thiazol-4-carboxylic acid methyl ester (2.6 g), tripotassium phosphate (3.8 g), dicyclohexyl(2′,6′-dimethoxybiphenyl-2-yl)phosphine (730 mg), DOX (30 mL), and water (6 mL) was added palladium (II) acetate (200 mg) at room temperature, and the mixture was stirred at 100° C. for 3 hours. After the mixture was allowed to cool to room temperature, ethyl acetate was added, and the mixture was washed with water and saturated aqueous sodium chloride solution. After the organic layer was dried over anhydrous magnesium sulfate, the insoluble materials were removed by filtration, and the filtrate was concentrated under reduced pressure. The resulting residue was purified by silica gel column chromatography (hexane/ethyl acetate) to give methyl 5-(4-{(1R)-1-[(tert-butoxycarbonyl)amino]-2-hydroxyethyl}phenyl)-1,3-thiazol-4-Carboxylate (1.48 g) as a solid.

### Production Example 104

Under nitrogen atmosphere, to a CH₂Cl₂ (20 mL) solution of methyl 5-(4-{(1R)-1-[(tert-butoxycarbonyl)amino]-2-hydroxyethyl}phenyl)-1,3-thiazol-4-carboxylate (1.01 g) was added diisobutylaluminum hydride (1M toluene solution, 11 mL) dropwise under ice-bath cooling, and the mixture was stirred under ice-bath cooling for 1 hour. Under ice-bath cooling, the reaction was quenched with MeOH, and 10% aqueous sodium potassium tartrate solution (60 mL) and CHCl₃ were added, and the mixture was stirred overnight. The mixture was divided into layers, the aqueous layer was extracted with CHCl₃, and the organic layer was dried over anhydrous sodium sulfate. The insoluble materials were removed by filtration, and the filtrate was concentrated under reduced pressure. The residue was dissolved in MeOH (10 mL), and sodium borohydride (350 mg) was added under ice-bath cooling. The mixture was stirred under ice-bath cooling for 1 hour. Water was added and the mixture was extracted with CHCl₃. The organic layer was dried over anhydrous sodium sulfate. The insoluble materials were removed by filtration, and the filtrate was concentrated under reduced pressure. The residue was purified by silica gel column chromatography (CHCl₃/MeOH) to give tert-butyl [(1R)-2-hydroxy-1-{4-[4-(hydroxymethyl)-1,3-thiazol-5-yl]phenyl}ethyl]carbamate (588 mg) as a solid.

### Production Example 106

To a mixture of tert-butyl N-[(1R)-1-(4-bromophenyl)-2-hydroxyethyl]carbamate (1 g), 2,2-dimethoxypropane (3.3 mL), and acetone (15 mL) was added a boron trifluoride-diethyl ether complex (26 μL), and the mixture was stirred at room temperature for 1 hour. TEA (66 μL) was added to the mixture, and the mixture was stirred at room temperature for 10 minutes. The mixture was concentrated under reduced pressure, and the residue was purified by silica gel column chromatography (hexane/ethyl acetate) to give tert-butyl (4R)-4-(4-bromophenyl)-2,2-dimethyl-1,3-oxazolidine-3-carboxylate (1.09 g) as a solid.

### Production Example 107

To a DOX (1.69 mL) solution of tert-butyl (4R)-4-(4-bromophenyl)-2,2-dimethyl-1,3-oxazolidine-3-carboxylate (300 mg) and 1,3-oxazolidin-2-one (183 mg) were added copper (I) iodide (32 mg), racemic-(1R,2R)-cyclohexane-1,2-diamine (20 μL), and potassium carbonate (290 mg) at room temperature. Under microwave irradiation, the mixture was stirred for 2 hours at 140° C. and for 1 hour at 150° C. Ethyl acetate and water were added, and the mixture was filtered through celite pad. The filtrate was concentrated under reduced pressure, and the residue was purified by silica gel column chromatography (hexane/ethyl acetate) to give tert-butyl (4R)-2,2-dimethyl-4-[4-(2-oxo-1,3-oxazolidin-3-yl)phenyl]-1,3-oxazolidine-3-carboxylate (120 mg) as a solid.

### Production Example 109

To a THF (27 mL) solution of 1-(4-bromophenyl)-2-fluoroethanone (2.7 g) and(S)-2-methylpropnane-2-sulfinamide (3.03 g) was added tetraisopropyl orthotitanate (11.1 g), and the mixture was stirred at 40° C. for 12 hours. Under ice-bath cooling (0-5° C.), a BH₃-THF complex (1 M THF solution, 18.4 mL) was added, and the mixture was stirred for 2 hours. After the reaction was quenched with water, the mixture was filtered through celite pad, and the filtrate was extracted with ethyl acetate. The organic layer was washed with saturated aqueous sodium chloride solution, and dried over anhydrous sodium sulfate. Then, the insoluble materials were removed by filtration, and the solution was concentrated under reduced pressure. The resulting residue was purified by silica gel column chromatography (petroleum ether/ethyl acetate) to give (S)—N-[(1R)-1-(4-bromophenyl)-2-fluoroethyl]-2-methylpropane-2-sulfinamide (3.2 g) as an oil.

In the same manner as in the production methods of the Production Examples described above, compounds shown in the tables presented later were produced. In addition, the production method, structure, and physiochemical data of the compound of each Production Example are shown in the tables presented later.

### Example 8

To a mixture of tert-butyl (1S,4S)-5-{6-cyclopropyl-7-[6-fluoro-5-methyl-1-(oxan-2-yl)-1H-indazol-4-yl]-8-{[4-(1-{(2S)-1-[(2S,4R)-4-hydroxy-2-({(1R)-2-hydroxy-1-[4-(4-methyl-1,3-thiazol-5-yl)phenyl]ethyl}carbamoyl) pyrrolidin-1-yl]-3-methyl-1-oxobutan-2-yl}-1H-1,2,3-triazol-4-yl)phenyl]methoxy}-2-[(oxan-4-yl)oxy]quinazolin-4-yl}-2,5-diazabicyclo[2.2.1]heptane-2-carboxylate (5.61 g) and CH₂Cl₂ (60 mL) was added trifluoroacetic acid (27 mL) under cooling (internal temperature: −5° C. or lower), and then the mixture was stirred at room temperature for 2 hours. The resulting reaction mixture was concentrated under reduced pressure, and saturated aqueous sodium hydrogen carbonate solution was added to the residue. The mixture was extracted three times with CHCl₃/MeOH (5/1), and then, the combined organic layer was dried over anhydrous sodium sulfate. The solution was concentrated under reduced pressure, and the resulting crude product was purified by ODS column chromatography (MeCN/0.1% aqueous formic acid solution). Saturated aqueous sodium hydrogen carbonate solution was added to fractions containing the target compound, and the mixture was extracted three times with CHCl₃/MeOH (5/1). The combined organic layer was dried over anhydrous sodium sulfate, and the solution was concentrated under reduced pressure. The residue was purified by silica gel column chromatography (basic silica gel, CHCl₃/MeOH) to give a product. Isopropyl acetate (70 mL) was added to the obtained product, and the mixture was stirred at 80° C. for 10 minutes, and was stirred at room temperature overnight. Hexane (70 mL) was added, and the mixture was stirred at room temperature for 1 hour. Then, the resulting solid was filtered, washed with isopropyl acetate/hexane (1/1), and dried under reduced pressure at 40° C. overnight to give (4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide (3.01 g) as a solid.

### Example 7

(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide (1.04 g) was dissolved in CH₂Cl₂ (9 mL) and MeOH (9 mL), hydrogen chloride (4M DOX solution, 3 mL) was then added under ice-bath cooling, and the mixture was stirred under ice-bath cooling for 30 minutes. The reaction mixture was concentrated under reduced pressure, and diethyl ether was added to the resulting residue. The precipitated solid was filtered, and dried under reduced pressure to give (4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide n-hydrochloride (1.04 g) as a solid.

### Example 20

To a mixture of tert-butyl (1S,4S)-5-{6-cyclopropyl-7-[6-fluoro-5-methyl-1-(oxan-2-yl)-1H-indazol-4-yl]-8-{[4-(1-{(2S)-1-[(2S,4R)-4-hydroxy-2-({(1R)-2-hydroxy-1-[4-(2-oxo-1,3-oxazolidin-3-yl)phenyl]ethyl}carbamoyl) pyrrolidin-1-yl]-3-methyl-1-oxobutan-2-yl}-1H-1,2,3-triazol-4-yl)phenyl]methoxy}-2-[(oxan-4-yl)oxy]quinazolin-4-yl}-2,5-diazabicyclo[2.2.1]heptane-2-carboxylate (170 mg), CH₂Cl₂ (2 mL), and MeOH (2 mL) was added hydrogen chloride (4M DOX solution, 0.988 mL) under ice-bath cooling, and the mixture was stirred at room temperature for 3 hours. The mixture was concentrated under reduced pressure, CHCl₃ and saturated aqueous sodium hydrogen carbonate solution were added, and the mixture was stirred for a while. Then, the aqueous layer was extracted with CHCl₃/MeOH (5/1), and the combined organic layer was dried over anhydrous sodium sulfate. The insoluble materials were removed by filtration, and the filtrate was concentrated under reduced pressure. The residue was purified by silica gel column chromatography (basic silica gel, CHCl₃/MeOH), and then purified by ODS column chromatography (MeCN/0.1% aqueous formic acid solution). The fractions containing the target compound were combined, basicified with saturated aqueous sodium hydrogen carbonate solution, and then extracted twice with CHCl₃/MeOH (5/1). The combined organic layer was dried over anhydrous sodium sulfate. The insoluble materials were removed by filtration, and the filtrate was concentrated under reduced pressure. The resulting solid was washed with diethyl ether, and dried under reduced pressure to give (4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(2-oxo-1,3-Oxazolidin-3-yl)phenyl]ethyl}-L-prolinamide (74 mg) as a solid.

### Example 18

Under nitrogen atmosphere, to a mixture of (4R)-1-[(2S)-2-(4-{4-[({4-[(1S,4S)-5-(tert-butoxycarbonyl)-2,5-diazabicyclo[2.2.1]heptan-2-yl]-6-cyclopropyl-7-[6-fluoro-5-methyl-1-(oxan-2-yl)-1H-indazol-4-yl]-2-[(oxan-4-yl)oxy]quinazolin-8-yl}oxy)methyl]phenyl}-1H-1,2,3-triazol-1-yl)-3-methylbutanoyl]-4-hydroxy-L-proline (65 mg), (2R)-2-amino-2-{4-[4-(hydroxymethyl)-1,3-thiazol-5-yl]phenyl}ethan-1-ol n-hydrochloride (25 mg), and DMF (1 mL) were sequentially added DIPEA (50 μL) and HATU (35 mg) under ice-bath cooling, and the mixture was stirred at room temperature for 1 hour. Water was added and the mixture was extracted with ethyl acetate. The organic layer was washed with water and saturated aqueous sodium chloride solution, and then dried over anhydrous sodium sulfate. The insoluble materials were removed by filtration, and the filtrate was concentrated under reduced pressure. The resulting residue was purified by silica gel column chromatography (CHCl₃/MeOH) to give an amide product (59 mg). Subsequently, the obtained compound was dissolved in CH₂Cl₂ (0.5 mL) and MeOH (0.5 mL), and hydrogen chloride (4M DOX solution, 0.5 mL) was added under ice-bath cooling. The mixture was stirred at room temperature for 2 hours, and then concentrated under reduced pressure. Diethyl ether was added to the resulting residue, and the precipitated solid was filtered, washed with diethyl ether, and dried under reduced pressure to give (4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-[(1R)-2-hydroxy-1-{4-[4-(hydroxymethyl)-1,3-thiazol-5-yl]phenyl}ethyl]-L-prolinamide n-hydrochloride (43 mg) as a solid.

### Example 48

(4R)-1-[(2)-2-(4-{4-[{6-cyclopropyl-4-[(1S,4S)-2,5-diazabicyclo[2.2.1]heptan-2-yl]-7-(6-fluoro-5-methyl-1H-indazol-4-yl)-2-[(2S)-2-methoxypropoxy]quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide was produced in the same method described in WO2022/173032.

### Example 70

(4R)-1-[(2)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(2S)-2-methoxypropoxy]quinazolin-8-yl}oxy)methyl]phenyl}-1H-1,2,3-triazol-1-yl)-3-methylbutanoyl]-N-{(1R)-1-[4-(1-ethyl-1H-pyrazol-5-yl)phenyl]-2-hydroxyethyl}-4-hydroxy-L-prolinamide was produced in the same method described in WO2022/173032.

In the same manner as in the production methods of the Examples described above, compounds shown in the tables presented later were produced. In addition, the structure and the physiochemical data of the compound of each Example is shown in the tables presented later.

In the tables presented below, the following abbreviations are sometimes used.
PEx: Production Example No., Ex: Example No., PSyn: Production Example No. produced in the same method, Syn: Example No. produced in the same method (for example, Syn: 8 represents that it was produced in the same method as for Example 8, and Syn: 18 #represents that it was obtained by desalting of a hydrochloride salt produced by the same method as for Example 18), Str: chemical structural formula (a compound with “*” in the chemical structural formula represents that the compound is single structure with regard to the axial chirality or central chirality). n HCl: n-hydrochloride (the compound with Production Example No. shows that the compound is monohydrochloride to trihydrochloride, and the compound with Example No. shows that the compound is monohydrochloride to pentahydrochloride), DAT: physiochemical data, ESI+: m/z value in mass spectrometry (ionization method ESI, [M+H]⁺ unless otherwise specified), ESI−: m/z value in mass spectrometry (ionization method ESI, [M−H]⁻ unless otherwise specified), NMR: δ value (ppm) of peak in 1H-NMR (500 MHZ) in DMSO-d₆, NMR (100° C.): δ value (ppm) of peak in 2H-NMR (500 MHZ) in DMSO-d₆ at 100° C., s: singlet (spectrum), d: doublet (spectrum), dd: double doublet (spectrum), t: triplet (spectrum), q: quartet (spectrum), m: multiplet (spectrum), br: broad (spectrum) (example: br s).

TABLE 1-1

| PEx | PSyn | Str | DAT |
|---|---|---|---|
| 1 | 1 | 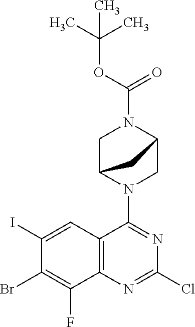 [SMILES: CC(C)(C)OC(=O)N1C[C@@H]2C[C@H]1CN2c1nc(Cl)nc2c(F)c(Br)c(I)cc12] | ESI+: 583.1, 585.1 |
| 2 | 2 | 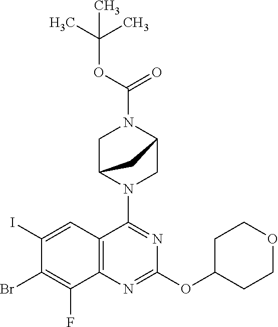 [SMILES: CC(C)(C)OC(=O)N1C[C@@H]2C[C@H]1CN2c1nc(OC2CCOCC2)nc2c(F)c(Br)c(I)cc12] | ESI+: 651.0 |
| 3 | 2 | 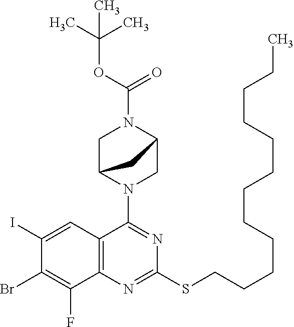 [SMILES: CCCCCCCCCCCCSc1nc(N2C[C@@H]3C[C@H]2CN3C(=O)OC(C)(C)C)c2cc(I)c(Br)c(F)c2n1] | ESI+: 751.3 |
| 4 | 2 | 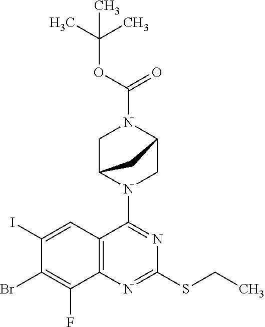 [SMILES: CCSc1nc(N2C[C@@H]3C[C@H]2CN3C(=O)OC(C)(C)C)c2cc(I)c(Br)c(F)c2n1] | ESI+: 610.9 |
| 5 | 5 | 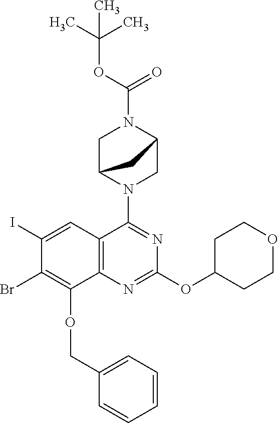 [SMILES: CC(C)(C)OC(=O)N1C[C@@H]2C[C@H]1CN2c1nc(OC2CCOCC2)nc2c(OCc3ccccc3)c(Br)c(I)cc12] | ESI+: 739.2 |
| 6 | 5 | 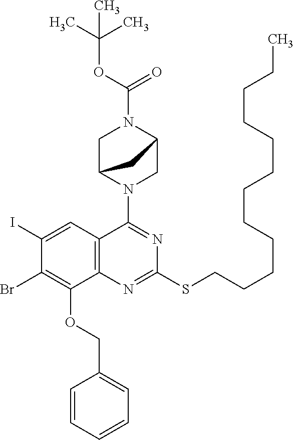 [SMILES: CCCCCCCCCCCCSc1nc(N2C[C@@H]3C[C@H]2CN3C(=O)OC(C)(C)C)c2cc(I)c(Br)c(OCc3ccccc3)c2n1] | ESI+: 837.5 |
| 7 | 5 | 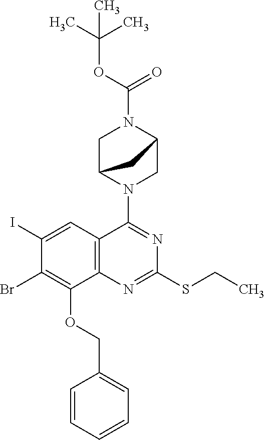 [SMILES: CCSc1nc(N2C[C@@H]3C[C@H]2CN3C(=O)OC(C)(C)C)c2cc(I)c(Br)c(OCc3ccccc3)c2n1] | ESI+: 698.9 |
| 8 | 8 | 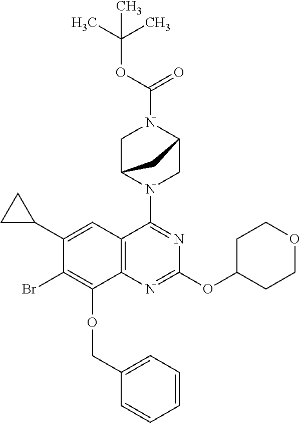 [SMILES: CC(C)(C)OC(=O)N1C[C@@H]2C[C@H]1CN2c1nc(OC2CCOCC2)nc2c(OCc3ccccc3)c(Br)c(C3CC3)cc12] | ESI+: 653.4 |
| 9 | 8 | 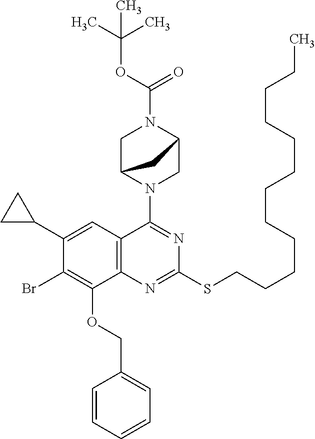 [SMILES: CCCCCCCCCCCCSc1nc(N2C[C@@H]3C[C@H]2CN3C(=O)OC(C)(C)C)c2cc(C3CC3)c(Br)c(OCc3ccccc3)c2n1] | ESI+: 753.5 |
| 10 | 8 | 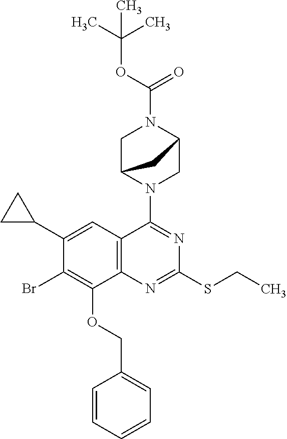 [SMILES: CCSc1nc(N2C[C@@H]3C[C@H]2CN3C(=O)OC(C)(C)C)c2cc(C3CC3)c(Br)c(OCc3ccccc3)c2n1] | ESI+: 613.3 |
| 11 | 11 | 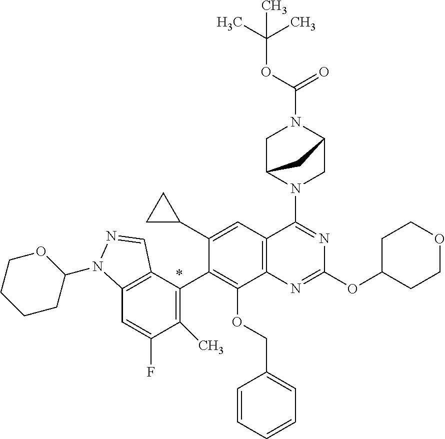 [SMILES: Cc1c(F)cc2c(cnn2C2CCCCO2)c1-c1c(C2CC2)cc2c(N3C[C@@H]4C[C@H]3CN4C(=O)OC(C)(C)C)nc(OC3CCOCC3)nc2c1OCc1ccccc1] | ESI+: 805.6 |
| 12 | 11 | 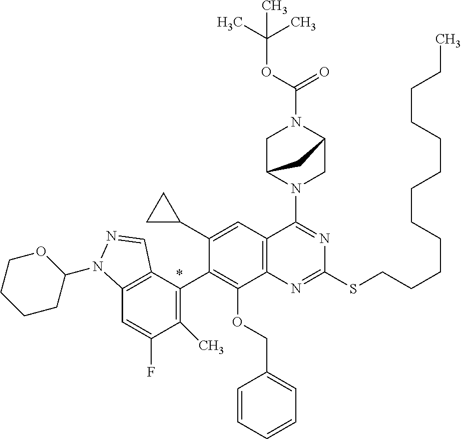 [SMILES: CCCCCCCCCCCCSc1nc(N2C[C@@H]3C[C@H]2CN3C(=O)OC(C)(C)C)c2cc(C3CC3)c(-c3c(C)c(F)cc4c3cnn4C3CCCCO3)c(OCc3ccccc3)c2n1] | ESI+: 905.5 |
| 13 | 11 | 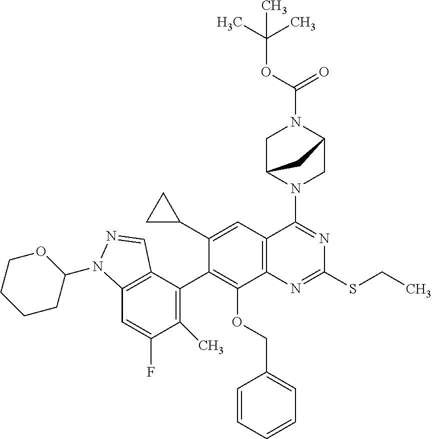 [SMILES: CCSc1nc(N2C[C@@H]3C[C@H]2CN3C(=O)OC(C)(C)C)c2cc(C3CC3)c(-c3c(C)c(F)cc4c3cnn4C3CCCCO3)c(OCc3ccccc3)c2n1] | ESI+: 765.4 |
| 14 | 14 | 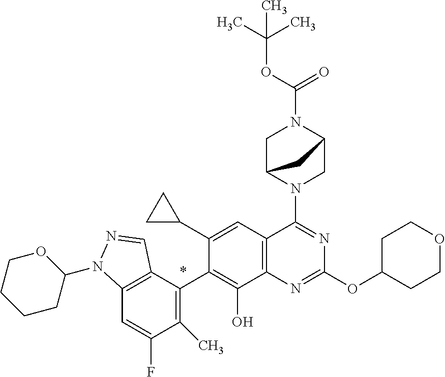 [SMILES: Cc1c(F)cc2c(cnn2C2CCCCO2)c1-c1c(C2CC2)cc2c(N3C[C@@H]4C[C@H]3CN4C(=O)OC(C)(C)C)nc(OC3CCOCC3)nc2c1O] | ESI+: 715.4 |
| 15 | 14 | 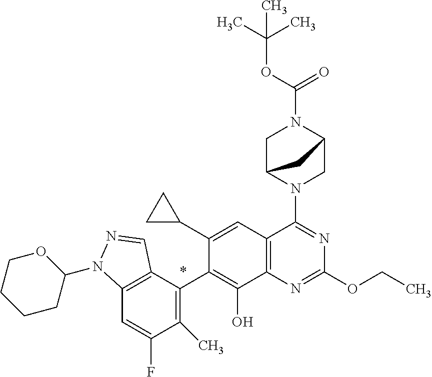 [SMILES: CCOc1nc(N2C[C@@H]3C[C@H]2CN3C(=O)OC(C)(C)C)c2cc(C3CC3)c(-c3c(C)c(F)cc4c3cnn4C3CCCCO3)c(O)c2n1] | ESI+: 659.4 |
| 16 | 14 | 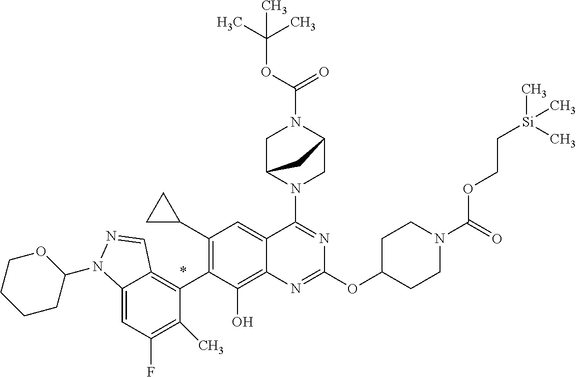 [SMILES: Cc1c(F)cc2c(cnn2C2CCCCO2)c1-c1c(C2CC2)cc2c(N3C[C@@H]4C[C@H]3CN4C(=O)OC(C)(C)C)nc(OC3CCN(C(=O)OCC[Si](C)(C)C)CC3)nc2c1O] | ESI+: 858.7 |
| 17 | 14 | 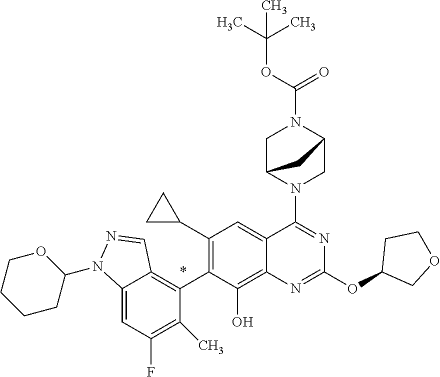 [SMILES: Cc1c(F)cc2c(cnn2C2CCCCO2)c1-c1c(C2CC2)cc2c(N3C[C@@H]4C[C@H]3CN4C(=O)OC(C)(C)C)nc(O[C@H]3CCOC3)nc2c1O] | ESI+: 701.4 |
| 18 | 14 | 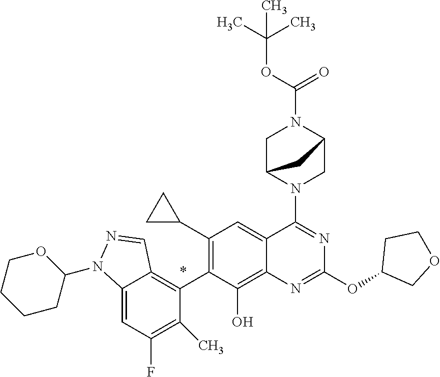 [SMILES: Cc1c(F)cc2c(cnn2C2CCCCO2)c1-c1c(C2CC2)cc2c(N3C[C@@H]4C[C@H]3CN4C(=O)OC(C)(C)C)nc(O[C@@H]3CCOC3)nc2c1O] | ESI+: 701.4 |
| 19 | 14 | 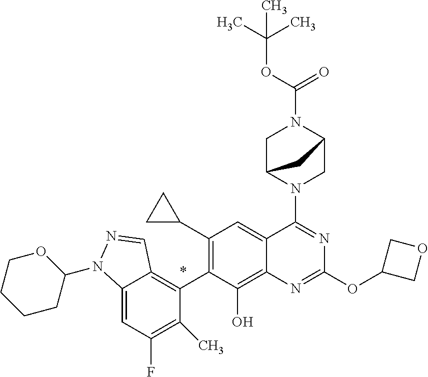 [SMILES: Cc1c(F)cc2c(cnn2C2CCCCO2)c1-c1c(C2CC2)cc2c(N3C[C@@H]4C[C@H]3CN4C(=O)OC(C)(C)C)nc(OC3COC3)nc2c1O] | ESI+; 687.4 |
| 20 | 14 | 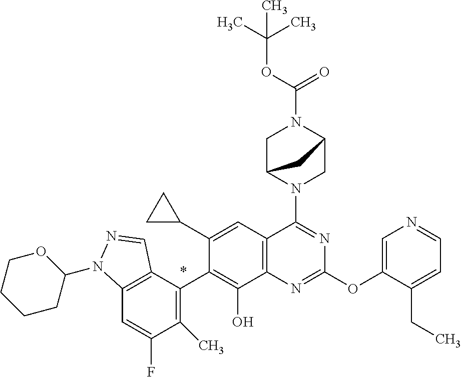 [SMILES: CCc1ccncc1Oc1nc(N2C[C@@H]3C[C@H]2CN3C(=O)OC(C)(C)C)c2cc(C3CC3)c(-c3c(C)c(F)cc4c3cnn4C3CCCCO3)c(O)c2n1] | ESI+: 736.5 |
| 21 | 14 | 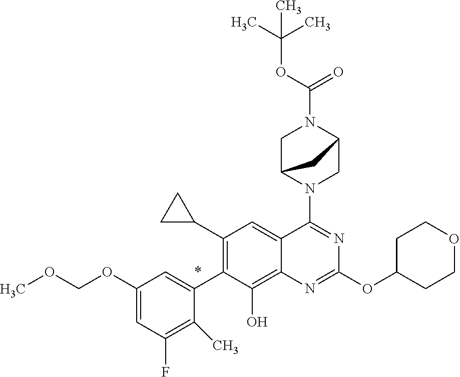 [SMILES: COCOc1cc(F)c(C)c(-c2c(C3CC3)cc3c(N4C[C@@H]5C[C@H]4CN5C(=O)OC(C)(C)C)nc(OC4CCOCC4)nc3c2O)c1] | ESI+: 651.6 |
| 22 | 22 | 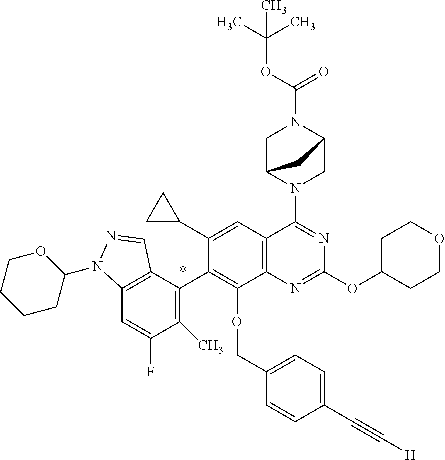 [SMILES: C#Cc1ccc(COc2c(-c3c(C)c(F)cc4c3cnn4C3CCCCO3)c(C3CC3)cc3c(N4C[C@@H]5C[C@H]4CN5C(=O)OC(C)(C)C)nc(OC4CCOCC4)nc23)cc1] | ESI+: 829.5 |
| 23 | 22 | 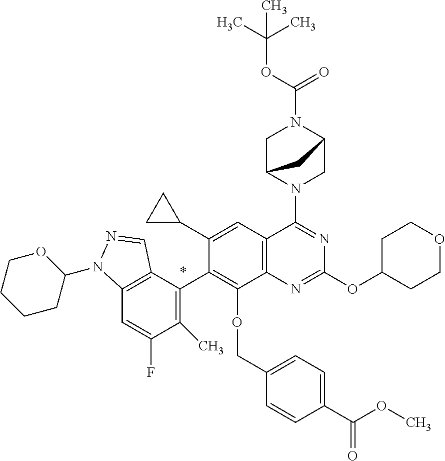 [SMILES: COC(=O)c1ccc(COc2c(-c3c(C)c(F)cc4c3cnn4C3CCCCO3)c(C3CC3)cc3c(N4C[C@@H]5C[C@H]4CN5C(=O)OC(C)(C)C)nc(OC4CCOCC4)nc23)cc1] | ESI+: 863.6 |
| 24 | 22 | 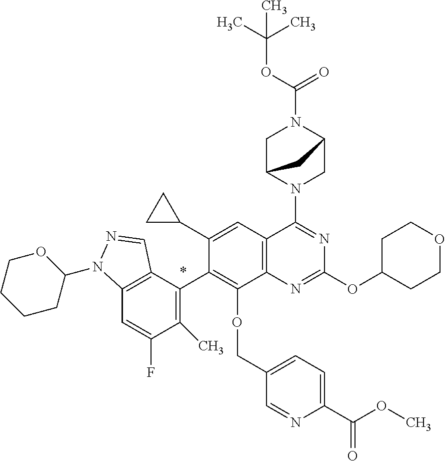 [SMILES: COC(=O)c1ccc(COc2c(-c3c(C)c(F)cc4c3cnn4C3CCCCO3)c(C3CC3)cc3c(N4C[C@@H]5C[C@H]4CN5C(=O)OC(C)(C)C)nc(OC4CCOCC4)nc23)cn1] | ESI+: 864.5 |
| 25 | 22 | 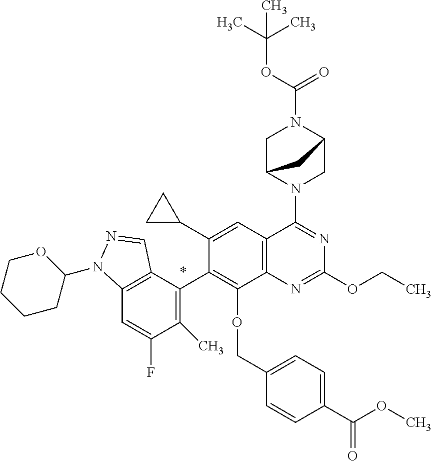 [SMILES: CCOc1nc(N2C[C@@H]3C[C@H]2CN3C(=O)OC(C)(C)C)c2cc(C3CC3)c(-c3c(C)c(F)cc4c3cnn4C3CCCCO3)c(OCc3ccc(C(=O)OC)cc3)c2n1] | ESI+: 807.6 |
| 26 | 22 | 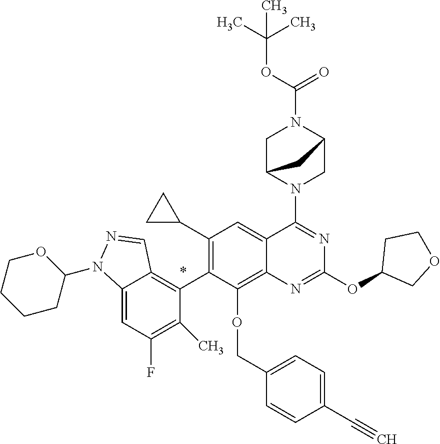 [SMILES: C#Cc1ccc(COc2c(-c3c(C)c(F)cc4c3cnn4C3CCCCO3)c(C3CC3)cc3c(N4C[C@@H]5C[C@H]4CN5C(=O)OC(C)(C)C)nc(O[C@H]4CCOC4)nc23)cc1] | ESI+: 815.7 |
| 27 | 22 | 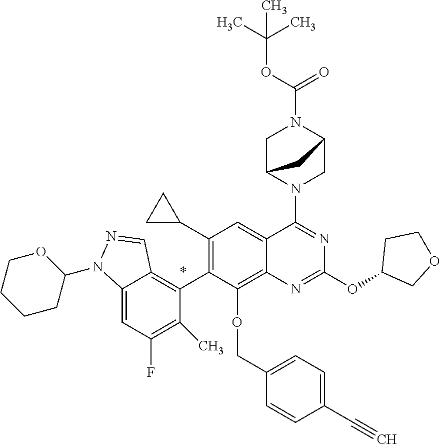 [SMILES: C#Cc1ccc(COc2c(-c3c(C)c(F)cc4c3cnn4C3CCCCO3)c(C3CC3)cc3c(N4C[C@@H]5C[C@H]4CN5C(=O)OC(C)(C)C)nc(O[C@@H]4CCOC4)nc23)cc1] | ESI+: 815.4 |
| 28 | 22 | 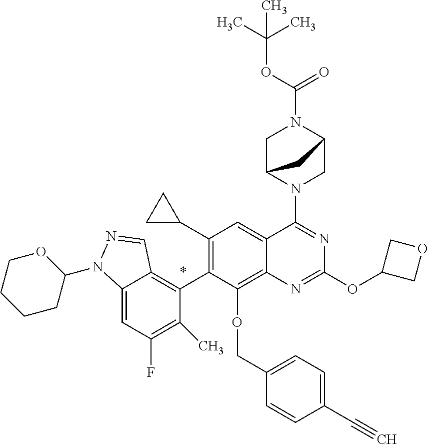 [SMILES: C#Cc1ccc(COc2c(-c3c(C)c(F)cc4c3cnn4C3CCCCO3)c(C3CC3)cc3c(N4C[C@@H]5C[C@H]4CN5C(=O)OC(C)(C)C)nc(OC4COC4)nc23)cc1] | ESI+: 801.4 |
| 29 | 22 | 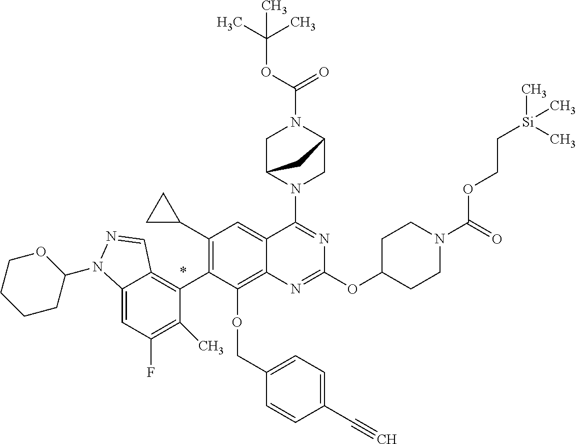 [SMILES: C#Cc1ccc(COc2c(-c3c(C)c(F)cc4c3cnn4C3CCCCO3)c(C3CC3)cc3c(N4C[C@@H]5C[C@H]4CN5C(=O)OC(C)(C)C)nc(OC4CCN(C(=O)OCC[Si](C)(C)C)CC4)nc23)cc1] | ESI+: 972.8 |
| 30 | 22 | 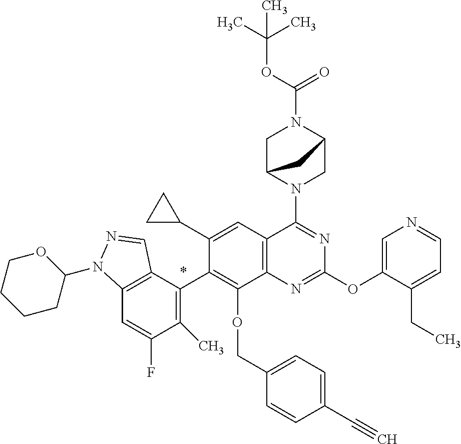 [SMILES: C#Cc1ccc(COc2c(-c3c(C)c(F)cc4c3cnn4C3CCCCO3)c(C3CC3)cc3c(N4C[C@@H]5C[C@H]4CN5C(=O)OC(C)(C)C)nc(Oc4cnccc4CC)nc23)cc1] | ESI+: 850.4 |
| 31 | 22 | 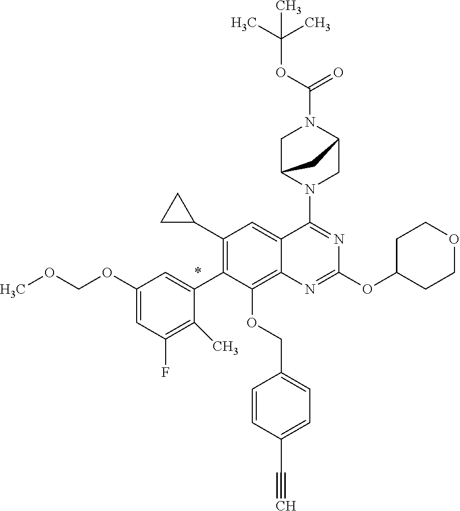 [SMILES: C#Cc1ccc(COc2c(-c3cc(OCOC)cc(F)c3C)c(C3CC3)cc3c(N4C[C@@H]5C[C@H]4CN5C(=O)OC(C)(C)C)nc(OC4CCOCC4)nc23)cc1] | ESI+; 765.5 |
| 32 | 32 | 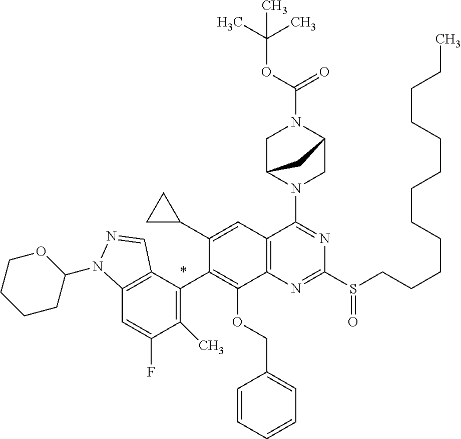 [SMILES: CCCCCCCCCCCCS(=O)c1nc(N2C[C@@H]3C[C@H]2CN3C(=O)OC(C)(C)C)c2cc(C3CC3)c(-c3c(C)c(F)cc4c3cnn4C3CCCCO3)c(OCc3ccccc3)c2n1] | ESI+: 921.4 |
| 33 | 32 | 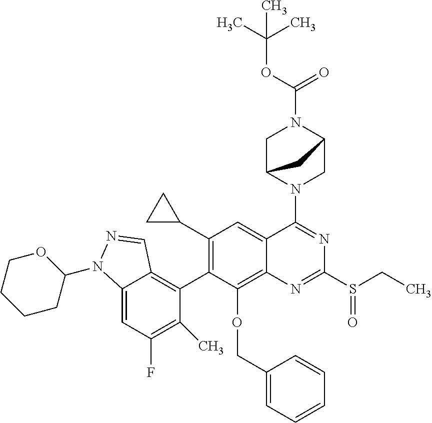 [SMILES: CCS(=O)c1nc(N2C[C@@H]3C[C@H]2CN3C(=O)OC(C)(C)C)c2cc(C3CC3)c(-c3c(C)c(F)cc4c3cnn4C3CCCCO3)c(OCc3ccccc3)c2n1] | ESI+: 781.4 |
| 34 | 34 | 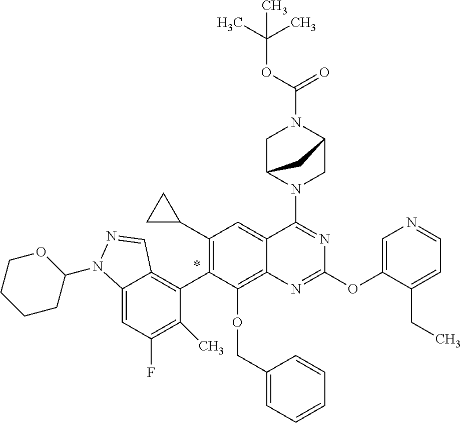 [SMILES: CCc1ccncc1Oc1nc(N2C[C@@H]3C[C@H]2CN3C(=O)OC(C)(C)C)c2cc(C3CC3)c(-c3c(C)c(F)cc4c3cnn4C3CCCCO3)c(OCc3ccccc3)c2n1] | ESI+: 826.5 |
| 35 | 34 | 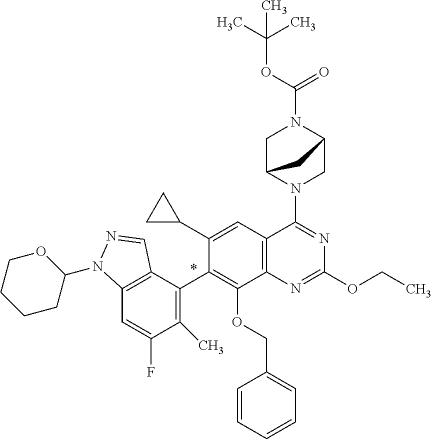 [SMILES: CCOc1nc(N2C[C@@H]3C[C@H]2CN3C(=O)OC(C)(C)C)c2cc(C3CC3)c(-c3c(C)c(F)cc4c3cnn4C3CCCCO3)c(OCc3ccccc3)c2n1] | ESI+: 749.5 |
| 36 | 34 | 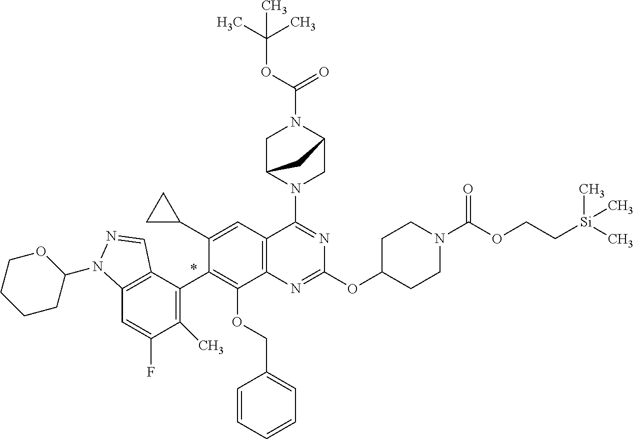 [SMILES: Cc1c(F)cc2c(cnn2C2CCCCO2)c1-c1c(C2CC2)cc2c(N3C[C@@H]4C[C@H]3CN4C(=O)OC(C)(C)C)nc(OC3CCN(C(=O)OCC[Si](C)(C)C)CC3)nc2c1OCc1ccccc1] | ESI+: 948.5 |
| 37 | 34 | 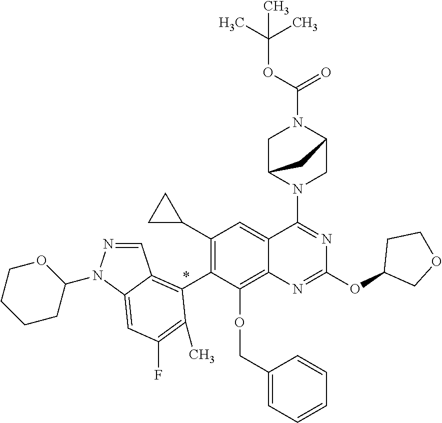 [SMILES: Cc1c(F)cc2c(cnn2C2CCCCO2)c1-c1c(C2CC2)cc2c(N3C[C@@H]4C[C@H]3CN4C(=O)OC(C)(C)C)nc(O[C@H]3CCOC3)nc2c1OCc1ccccc1] | ESI+: 791.4 |
| 38 | 34 | 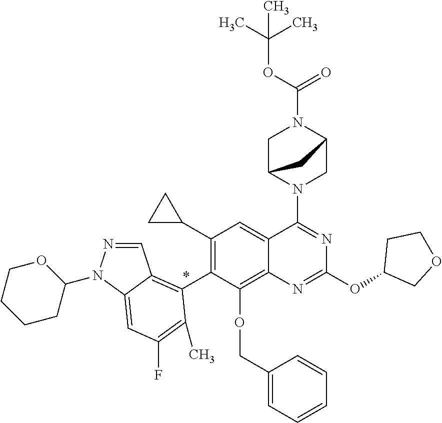 [SMILES: Cc1c(F)cc2c(cnn2C2CCCCO2)c1-c1c(C2CC2)cc2c(N3C[C@@H]4C[C@H]3CN4C(=O)OC(C)(C)C)nc(O[C@@H]3CCOC3)nc2c1OCc1ccccc1] | ESI+: 791.4 |
| 39 | 34 | 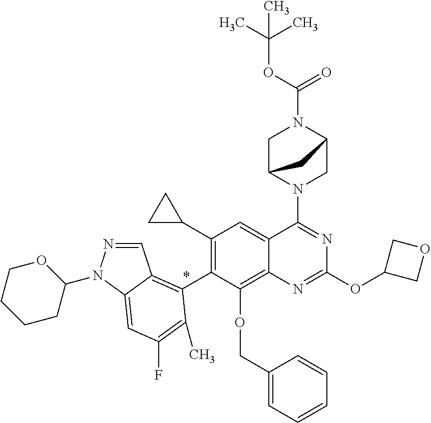 [SMILES: Cc1c(F)cc2c(cnn2C2CCCCO2)c1-c1c(C2CC2)cc2c(N3C[C@@H]4C[C@H]3CN4C(=O)OC(C)(C)C)nc(OC3COC3)nc2c1OCc1ccccc1] | ESI+: 777.4 |
| 40 | 40 | 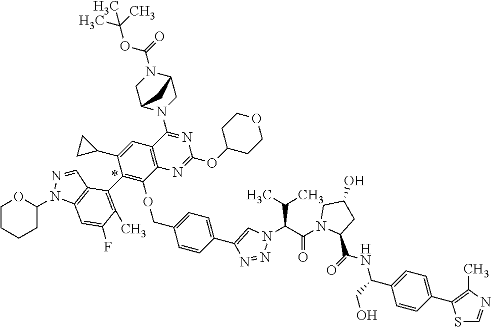 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6c5cnn6C5CCCCO5)c(C5CC5)cc5c(N6C[C@@H]7C[C@H]6CN7C(=O)OC(C)(C)C)nc(OC6CCOCC6)nc45)cc3)nn2)cc1] | ESI+: 1301.3 |
| 41 | 40 | 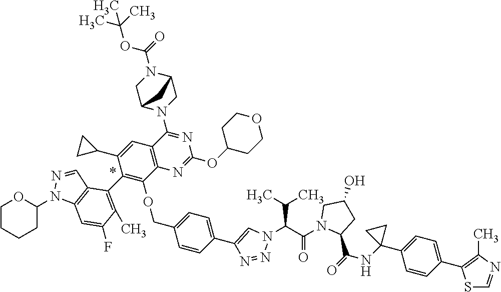 [SMILES: Cc1ncsc1-c1ccc(C2(NC(=O)[C@@H]3C[C@@H](O)CN3C(=O)[C@H](C(C)C)n3cc(-c4ccc(COc5c(-c6c(C)c(F)cc7c6cnn7C6CCCCO6)c(C6CC6)cc6c(N7C[C@@H]8C[C@H]7CN8C(=O)OC(C)(C)C)nc(OC7CCOCC7)nc56)cc4)nn3)CC2)cc1] | ESI+: 1297.5 |
| 42 | 40 | 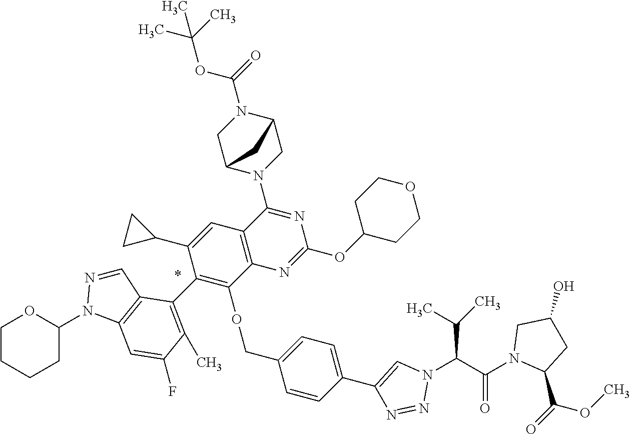 [SMILES: COC(=O)[C@@H]1C[C@@H](O)CN1C(=O)[C@H](C(C)C)n1cc(-c2ccc(COc3c(-c4c(C)c(F)cc5c4cnn5C4CCCCO4)c(C4CC4)cc4c(N5C[C@@H]6C[C@H]5CN6C(=O)OC(C)(C)C)nc(OC5CCOCC5)nc34)cc2)nn1] | ESI+: 1099.4 |
| 43 | 40 | 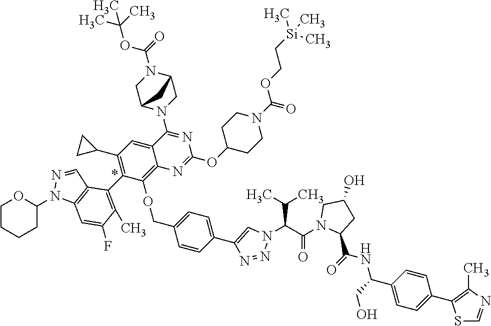 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6c5cnn6C5CCCCO5)c(C5CC5)cc5c(N6C[C@@H]7C[C@H]6CN7C(=O)OC(C)(C)C)nc(OC6CCN(C(=O)OCC[Si](C)(C)C)CC6)nc45)cc3)nn2)cc1] | ESI+: 1444.5 |
| 44 | 40 | 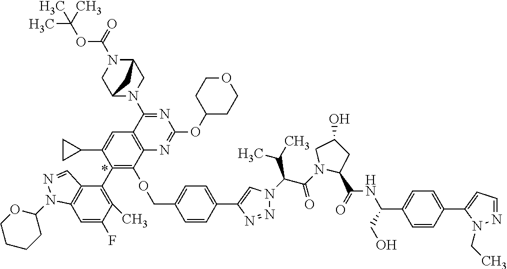 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6c5cnn6C5CCCCO5)c(C5CC5)cc5c(N6C[C@@H]7C[C@H]6CN7C(=O)OC(C)(C)C)nc(OC6CCOCC6)nc45)cc3)nn2)cc1] | ESI+: 1298.4 |
| 45 | 40 | 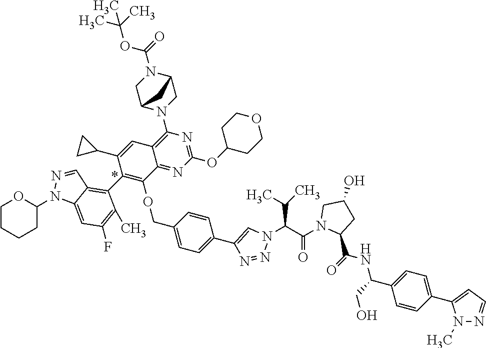 [SMILES: Cc1c(F)cc2c(cnn2C2CCCCO2)c1-c1c(C2CC2)cc2c(N3C[C@@H]4C[C@H]3CN4C(=O)OC(C)(C)C)nc(OC3CCOCC3)nc2c1OCc1ccc(-c2cn([C@H](C(=O)N3C[C@H](O)C[C@H]3C(=O)N[C@@H](CO)c3ccc(-c4ccnn4C)cc3)C(C)C)nn2)cc1] | ESI+: 1284.4 |
| 46 | 40 | 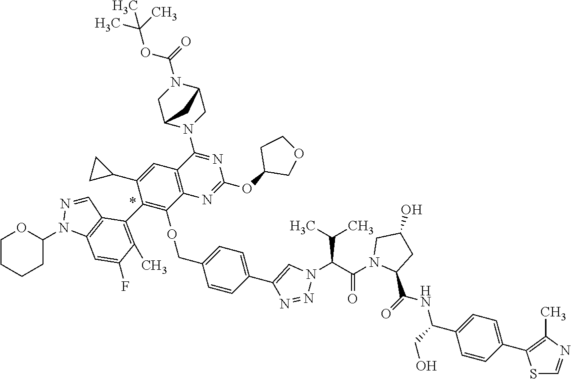 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6c5cnn6C5CCCCO5)c(C5CC5)cc5c(N6C[C@@H]7C[C@H]6CN7C(=O)OC(C)(C)C)nc(O[C@H]6CCOC6)nc45)cc3)nn2)cc1] | ESI+: 1287.3 |
| 47 | 40 | 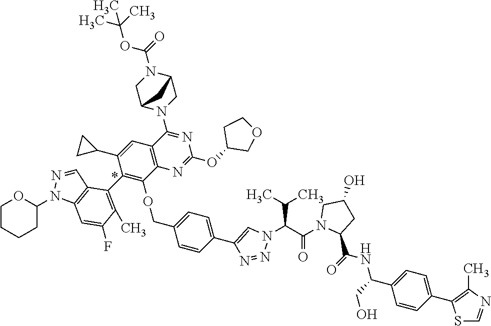 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6c5cnn6C5CCCCO5)c(C5CC5)cc5c(N6C[C@@H]7C[C@H]6CN7C(=O)OC(C)(C)C)nc(O[C@@H]6CCOC6)nc45)cc3)nn2)cc1] | ESI+: 1287.4 |
| 48 | 40 | 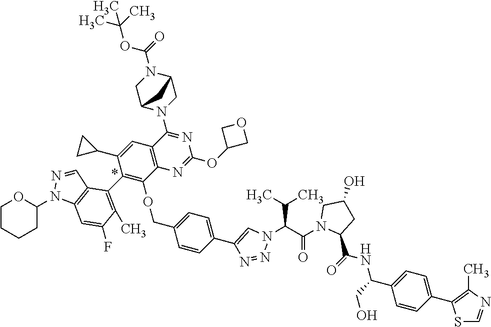 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6c5cnn6C5CCCCO5)c(C5CC5)cc5c(N6C[C@@H]7C[C@H]6CN7C(=O)OC(C)(C)C)nc(OC6COC6)nc45)cc3)nn2)cc1] | ESI+: 1274.0 |
| 49 | 40 | 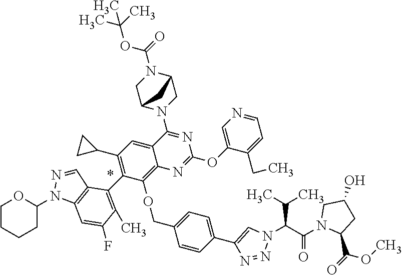 [SMILES: CCc1ccncc1Oc1nc(N2C[C@@H]3C[C@H]2CN3C(=O)OC(C)(C)C)c2cc(C3CC3)c(-c3c(C)c(F)cc4c3cnn4C3CCCCO3)c(OCc3ccc(-c4cn([C@H](C(=O)N5C[C@H](O)C[C@H]5C(=O)OC)C(C)C)nn4)cc3)c2n1] | ESI+: 1142.5 [M + Na]⁺ |
| 50 | 40 | 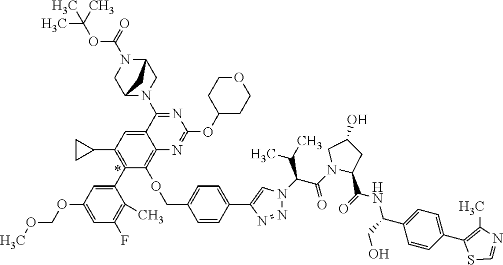 [SMILES: COCOc1cc(F)c(C)c(-c2c(C3CC3)cc3c(N4C[C@@H]5C[C@H]4CN5C(=O)OC(C)(C)C)nc(OC4CCOCC4)nc3c2OCc2ccc(-c3cn([C@H](C(=O)N4C[C@H](O)C[C@H]4C(=O)N[C@@H](CO)c4ccc(-c5scnc5C)cc4)C(C)C)nn3)cc2)c1] | ESI+: 1237.5 |
| 51 | 51 | 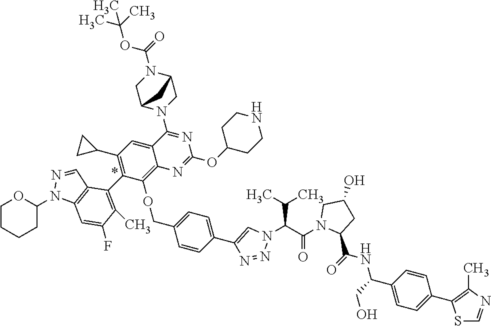 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6c5cnn6C5CCCCO5)c(C5CC5)cc5c(N6C[C@@H]7C[C@H]6CN7C(=O)OC(C)(C)C)nc(OC6CCNCC6)nc45)cc3)nn2)cc1] | ESI+: 1300.8 |
| 52 | 52 | 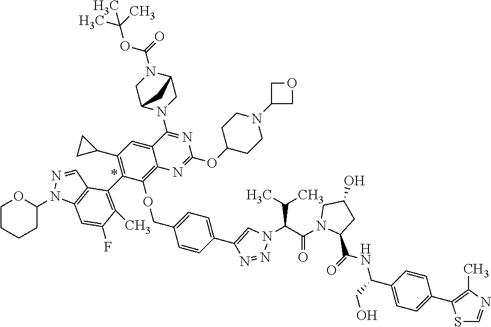 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6c5cnn6C5CCCCO5)c(C5CC5)cc5c(N6C[C@@H]7C[C@H]6CN7C(=O)OC(C)(C)C)nc(OC6CCN(C7COC7)CC6)nc45)cc3)nn2)cc1] | ESI+: 1356.5 |
| 53 | 53 | 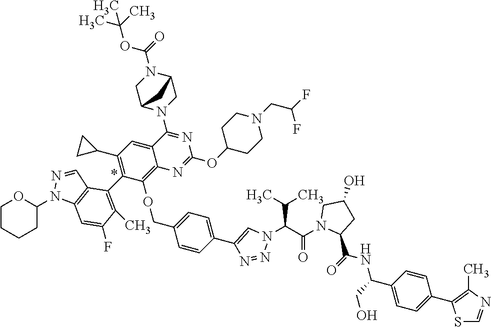 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6c5cnn6C5CCCCO5)c(C5CC5)cc5c(N6C[C@@H]7C[C@H]6CN7C(=O)OC(C)(C)C)nc(OC6CCN(CC(F)F)CC6)nc45)cc3)nn2)cc1] | ESI+: 1364.4 |
| 54 | 54 | 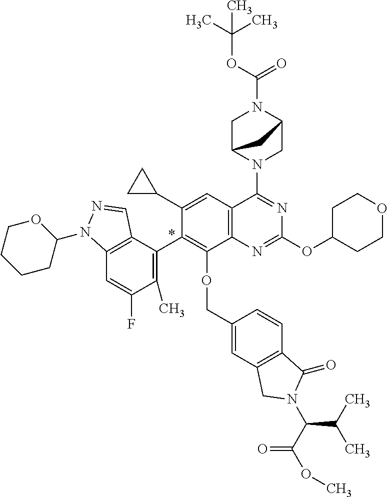 [SMILES: COC(=O)[C@H](C(C)C)N1Cc2cc(COc3c(-c4c(C)c(F)cc5c4cnn5C4CCCCO4)c(C4CC4)cc4c(N5C[C@@H]6C[C@H]5CN6C(=O)OC(C)(C)C)nc(OC5CCOCC5)nc34)ccc2C1=O] | ESI+: 974.4 |
| 55 | 54 | 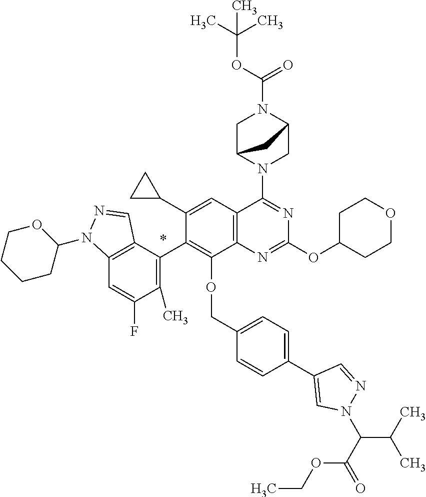 [SMILES: CCOC(=O)C(C(C)C)n1cc(-c2ccc(COc3c(-c4c(C)c(F)cc5c4cnn5C4CCCCO4)c(C4CC4)cc4c(N5C[C@@H]6C[C@H]5CN6C(=O)OC(C)(C)C)nc(OC5CCOCC5)nc34)cc2)cn1] | ESI+: 999.5 |
| 56 | 56 | 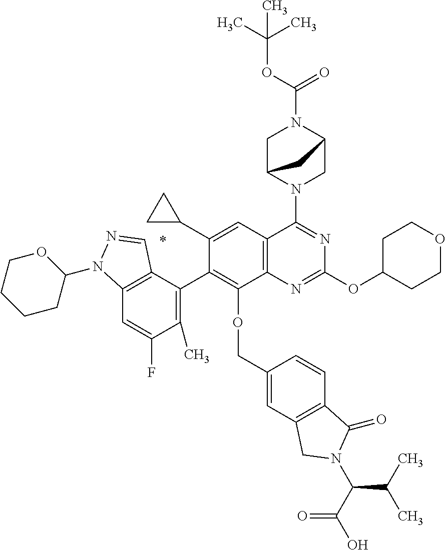 [SMILES: Cc1c(F)cc2c(cnn2C2CCCCO2)c1-c1c(C2CC2)cc2c(N3C[C@@H]4C[C@H]3CN4C(=O)OC(C)(C)C)nc(OC3CCOCC3)nc2c1OCc1ccc2c(c1)CN([C@H](C(=O)O)C(C)C)C2=O] | ESI+: 960.5 |
| 57 | 56 | 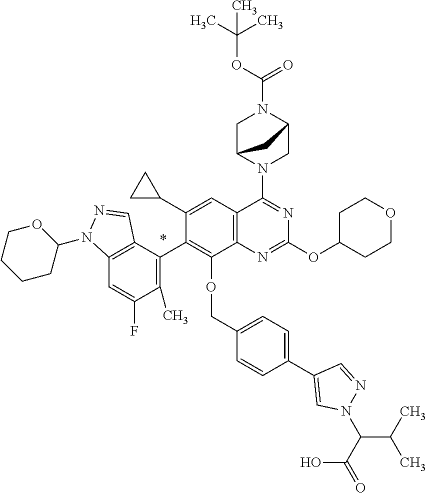 [SMILES: Cc1c(F)cc2c(cnn2C2CCCCO2)c1-c1c(C2CC2)cc2c(N3C[C@@H]4C[C@H]3CN4C(=O)OC(C)(C)C)nc(OC3CCOCC3)nc2c1OCc1ccc(-c2cnn(C(C(=O)O)C(C)C)c2)cc1] | ESI+: 969.7 |
| 58 | 56 | 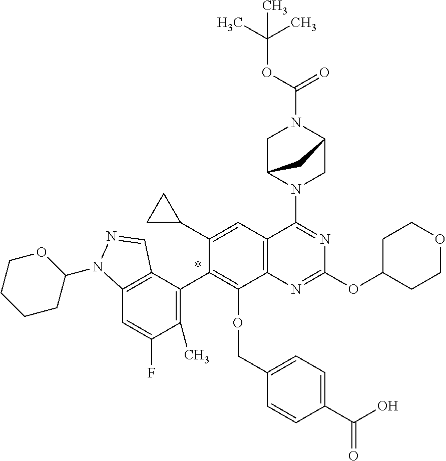 [SMILES: Cc1c(F)cc2c(cnn2C2CCCCO2)c1-c1c(C2CC2)cc2c(N3C[C@@H]4C[C@H]3CN4C(=O)OC(C)(C)C)nc(OC3CCOCC3)nc2c1OCc1ccc(C(=O)O)cc1] | ESI+: 849.5 |
| 59 | 56 | 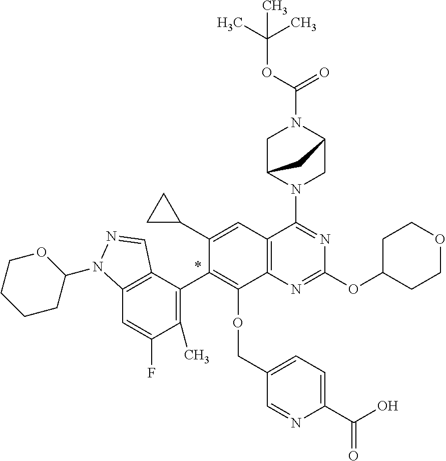 [SMILES: Cc1c(F)cc2c(cnn2C2CCCCO2)c1-c1c(C2CC2)cc2c(N3C[C@@H]4C[C@H]3CN4C(=O)OC(C)(C)C)nc(OC3CCOCC3)nc2c1OCc1ccc(C(=O)O)nc1] | ESI+: 850.4 |
| 60 | 56 | 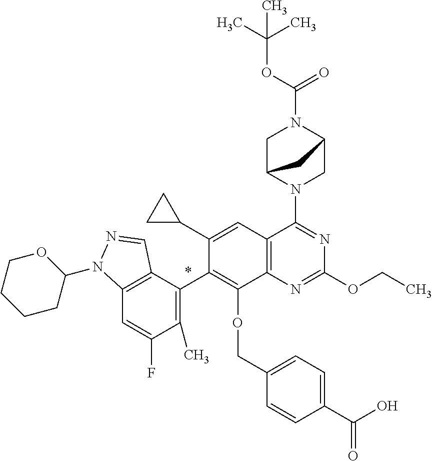 [SMILES: CCOc1nc(N2C[C@@H]3C[C@H]2CN3C(=O)OC(C)(C)C)c2cc(C3CC3)c(-c3c(C)c(F)cc4c3cnn4C3CCCCO3)c(OCc3ccc(C(=O)O)cc3)c2n1] | ESI+: 793.7 |
| 61 | 61 | 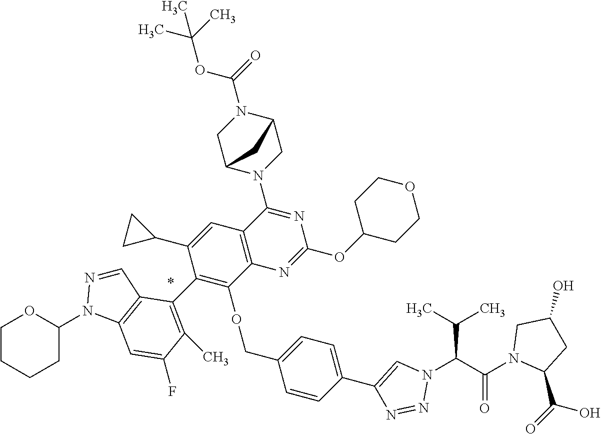 [SMILES: Cc1c(F)cc2c(cnn2C2CCCCO2)c1-c1c(C2CC2)cc2c(N3C[C@@H]4C[C@H]3CN4C(=O)OC(C)(C)C)nc(OC3CCOCC3)nc2c1OCc1ccc(-c2cn([C@H](C(=O)N3C[C@H](O)C[C@H]3C(=O)O)C(C)C)nn2)cc1] | ESI+: 1083.6 |
| 62 | 61 | 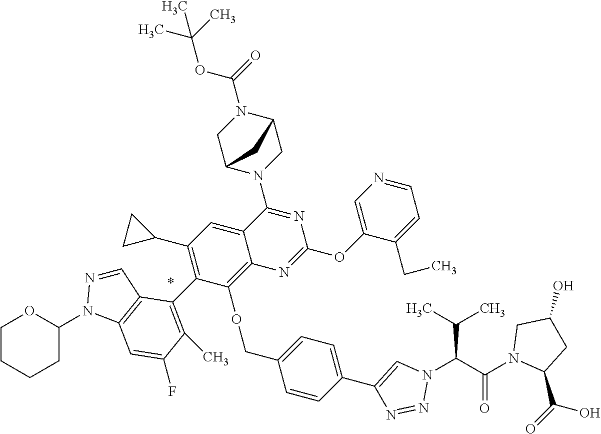 [SMILES: CCc1ccncc1Oc1nc(N2C[C@@H]3C[C@H]2CN3C(=O)OC(C)(C)C)c2cc(C3CC3)c(-c3c(C)c(F)cc4c3cnn4C3CCCCO3)c(OCc3ccc(-c4cn([C@H](C(=O)N5C[C@H](O)C[C@H]5C(=O)O)C(C)C)nn4)cc3)c2n1] | ESI+: 1104.5 |
| 63 | 63 | 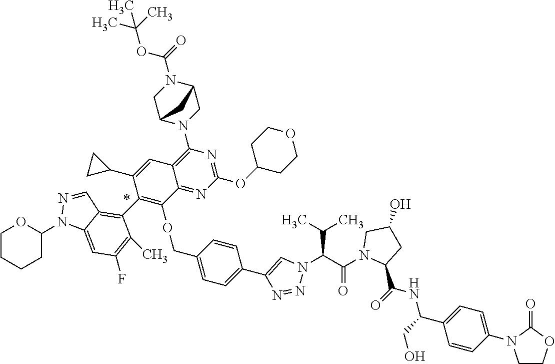 [SMILES: Cc1c(F)cc2c(cnn2C2CCCCO2)c1-c1c(C2CC2)cc2c(N3C[C@@H]4C[C@H]3CN4C(=O)OC(C)(C)C)nc(OC3CCOCC3)nc2c1OCc1ccc(-c2cn([C@H](C(=O)N3C[C@H](O)C[C@H]3C(=O)N[C@@H](CO)c3ccc(N4CCOC4=O)cc3)C(C)C)nn2)cc1] | ESI+: 1289.9 |
| 64 | 63 | 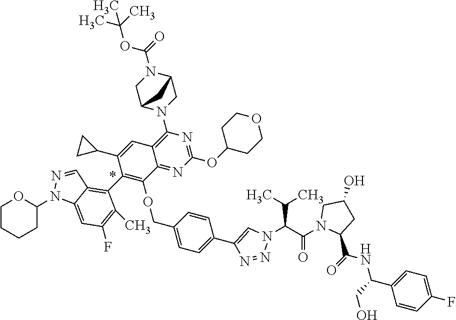 [SMILES: Cc1c(F)cc2c(cnn2C2CCCCO2)c1-c1c(C2CC2)cc2c(N3C[C@@H]4C[C@H]3CN4C(=O)OC(C)(C)C)nc(OC3CCOCC3)nc2c1OCc1ccc(-c2cn([C@H](C(=O)N3C[C@H](O)C[C@H]3C(=O)N[C@@H](CO)c3ccc(F)cc3)C(C)C)nn2)cc1] | ESI+: 1222.8 |
| 65 | 63 | 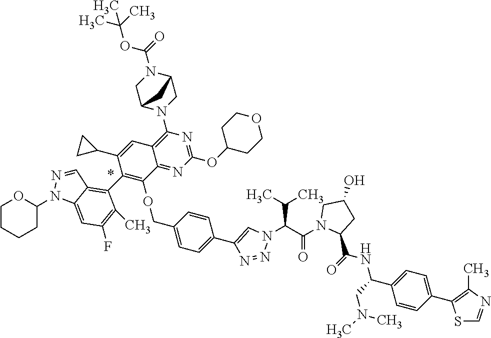 [SMILES: Cc1ncsc1-c1ccc([C@H](CN(C)C)NC(=O)[C@@H]2C[C@@H](O)CN2C(=O)[C@H](C(C)C)n2cc(-c3ccc(COc4c(-c5c(C)c(F)cc6c5cnn6C5CCCCO5)c(C5CC5)cc5c(N6C[C@@H]7C[C@H]6CN7C(=O)OC(C)(C)C)nc(OC6CCOCC6)nc45)cc3)nn2)cc1] | ESI+: 1329.2 |
| 66 | 66 | 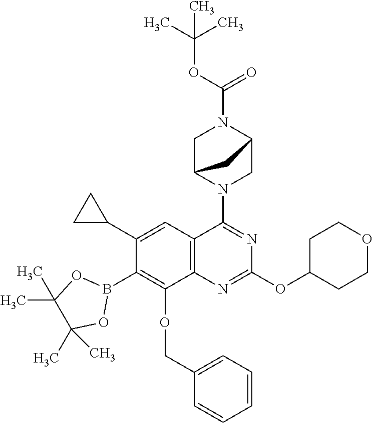 [SMILES: CC(C)(C)OC(=O)N1C[C@@H]2C[C@H]1CN2c1nc(OC2CCOCC2)nc2c(OCc3ccccc3)c(B3OC(C)(C)C(C)(C)O3)c(C3CC3)cc12] | ESI+: 699.5 |
| 67 | 67 | 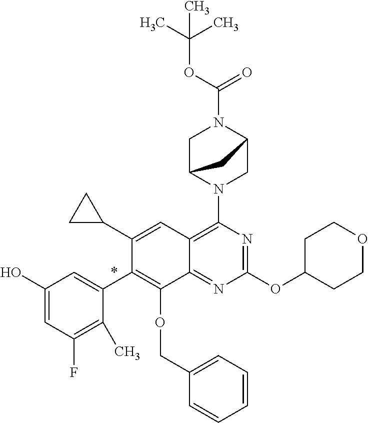 [SMILES: Cc1c(F)cc(O)cc1-c1c(C2CC2)cc2c(N3C[C@@H]4C[C@H]3CN4C(=O)OC(C)(C)C)nc(OC3CCOCC3)nc2c1OCc1ccccc1] | ESI+: 697.6 |
| 68 | 68 | 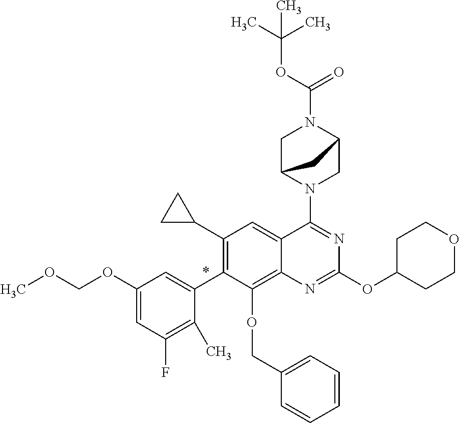 [SMILES: COCOc1cc(F)c(C)c(-c2c(C3CC3)cc3c(N4C[C@@H]5C[C@H]4CN5C(=O)OC(C)(C)C)nc(OC4CCOCC4)nc3c2OCc2ccccc2)c1] | ESI+: 741.7 |
| 69 | 69 | 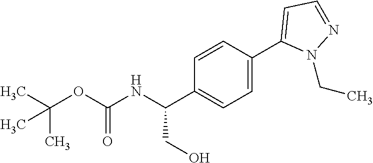 [SMILES: CCn1nccc1-c1ccc([C@H](CO)NC(=O)OC(C)(C)C)cc1] | ESI+: 332.2 |
| 70 | 69 | 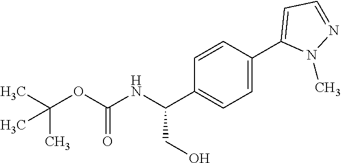 [SMILES: Cn1nccc1-c1ccc([C@H](CO)NC(=O)OC(C)(C)C)cc1] | ESI+: 318.2 |
| 71 | 71 | 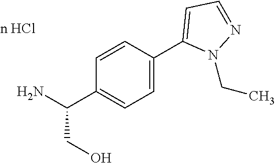 [SMILES: CCn1nccc1-c1ccc([C@@H](N)CO)cc1] | ESI+: 232.2 |
| 72 | 71 | 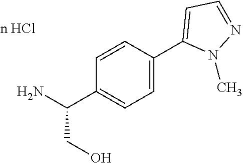 [SMILES: Cn1nccc1-c1ccc([C@@H](N)CO)cc1] | ESI+: 218.1 |
| 73 | 73 | 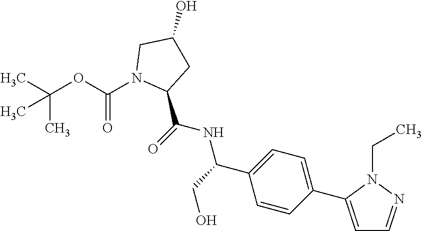 [SMILES: CCn1nccc1-c1ccc([C@H](CO)NC(=O)[C@@H]2C[C@@H](O)CN2C(=O)OC(C)(C)C)cc1] | ESI+: 445.3 |
| 74 | 73 | 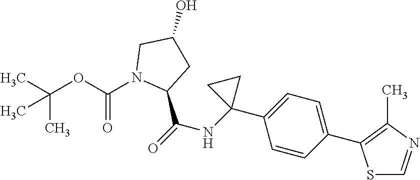 [SMILES: Cc1ncsc1-c1ccc(C2(NC(=O)[C@@H]3C[C@@H](O)CN3C(=O)OC(C)(C)C)CC2)cc1] | ESI+: 444.4 |
| 75 | 73 | 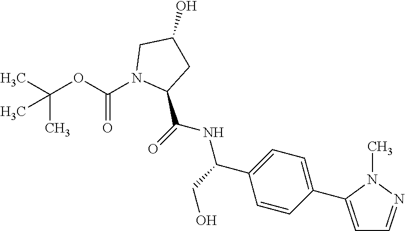 [SMILES: Cn1nccc1-c1ccc([C@H](CO)NC(=O)[C@@H]2C[C@@H](O)CN2C(=O)OC(C)(C)C)cc1] | ESI+: 431.3 |
| 76 | 76 | 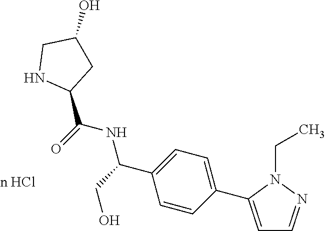 [SMILES: CCn1nccc1-c1ccc([C@H](CO)NC(=O)[C@@H]2C[C@@H](O)CN2)cc1] | ESI+: 345.2 |
| 77 | 76 | 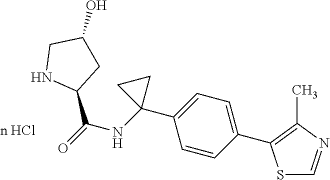 [SMILES: Cc1ncsc1-c1ccc(C2(NC(=O)[C@@H]3C[C@@H](O)CN3)CC2)cc1] | ESI+: 344.3 |
| 78 | 76 | 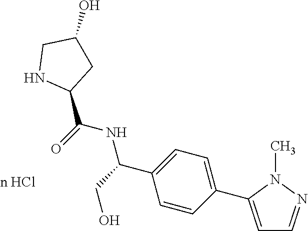 [SMILES: Cn1nccc1-c1ccc([C@H](CO)NC(=O)[C@@H]2C[C@@H](O)CN2)cc1] | ESI+: 331.2 |
| 79 | 79 | 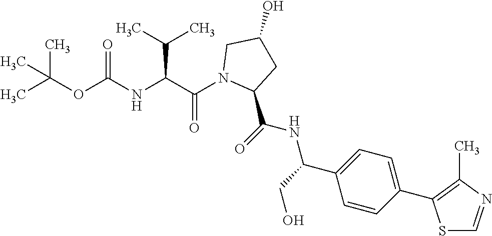 [SMILES: Cc1ncsc1-c1ccc([C@H](CO)NC(=O)[C@@H]2C[C@@H](O)CN2C(=O)[C@@H](NC(=O)OC(C)(C)C)C(C)C)cc1] | ESI+: 547.4 |
| 80 | 79 | 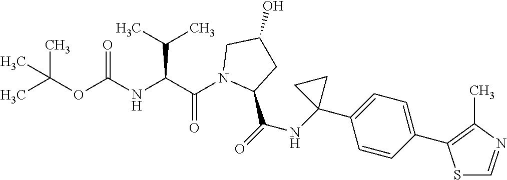 [SMILES: Cc1ncsc1-c1ccc(C2(NC(=O)[C@@H]3C[C@@H](O)CN3C(=O)[C@@H](NC(=O)OC(C)(C)C)C(C)C)CC2)cc1] | ESI+: 565.4 [M + Na]+ |
| 81 | 79 | 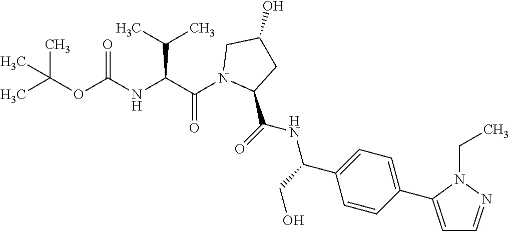 [SMILES: CCn1nccc1-c1ccc([C@H](CO)NC(=O)[C@@H]2C[C@@H](O)CN2C(=O)[C@@H](NC(=O)OC(C)(C)C)C(C)C)cc1] | ESI+: 544.3 |
| 82 | 79 | 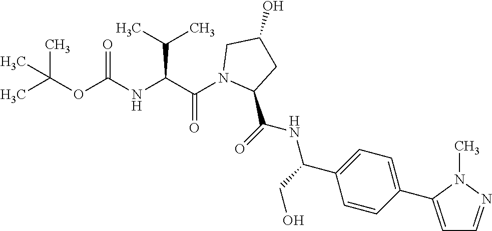 [SMILES: CC(C)[C@H](NC(=O)OC(C)(C)C)C(=O)N1C[C@H](O)C[C@H]1C(=O)N[C@@H](CO)c1ccc(-c2ccnn2C)cc1] | ESI+: 530.4 |
| 83 | 83 | 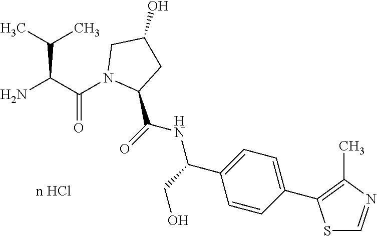 [SMILES: Cc1ncsc1-c1ccc([C@H](CO)NC(=O)[C@@H]2C[C@@H](O)CN2C(=O)[C@@H](N)C(C)C)cc1] | ESI+: 447.3 |
| 84 | 83 | 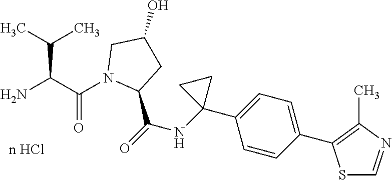 [SMILES: Cc1ncsc1-c1ccc(C2(NC(=O)[C@@H]3C[C@@H](O)CN3C(=O)[C@@H](N)C(C)C)CC2)cc1] | ESI+: 443.4 |
| 85 | 83 | 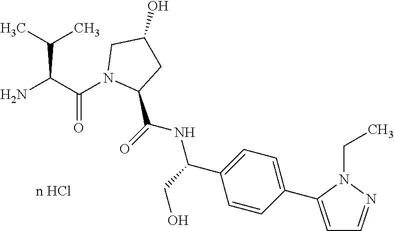 [SMILES: CCn1nccc1-c1ccc([C@H](CO)NC(=O)[C@@H]2C[C@@H](O)CN2C(=O)[C@@H](N)C(C)C)cc1] | ESI+: 444.3 |
| 86 | 83 | 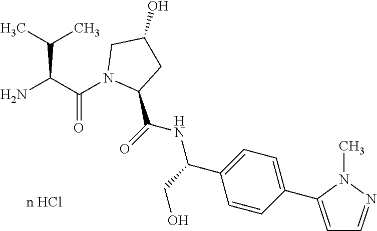 [SMILES: CC(C)[C@H](N)C(=O)N1C[C@H](O)C[C@H]1C(=O)N[C@@H](CO)c1ccc(-c2ccnn2C)cc1] | ESI+: 430.3 |
| 87 | 83 | 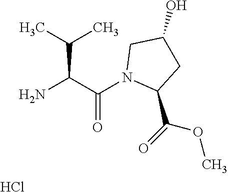 [SMILES: COC(=O)[C@@H]1C[C@@H](O)CN1C(=O)[C@@H](N)C(C)C ; Cl] | ESI+: 245.2 |
| 88 | 88 | 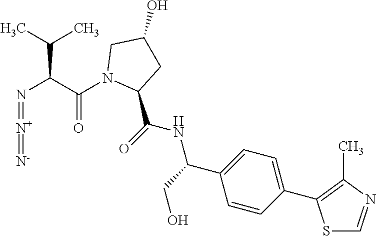 [SMILES: Cc1ncsc1-c1ccc([C@H](CO)NC(=O)[C@@H]2C[C@@H](O)CN2C(=O)[C@@H](N=[N+]=[N-])C(C)C)cc1] | ESI+: 473.3 |
| 89 | 88 | 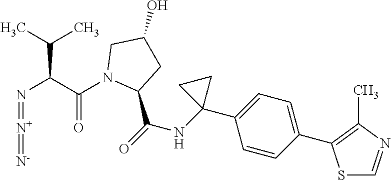 [SMILES: Cc1ncsc1-c1ccc(C2(NC(=O)[C@@H]3C[C@@H](O)CN3C(=O)[C@@H](N=[N+]=[N-])C(C)C)CC2)cc1] | ESI+: 469.3 |
| 90 | 88 | 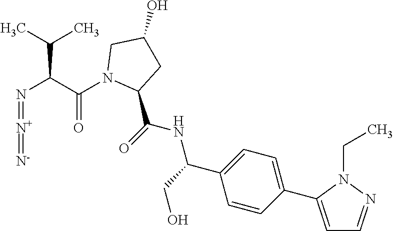 [SMILES: CCn1nccc1-c1ccc([C@H](CO)NC(=O)[C@@H]2C[C@@H](O)CN2C(=O)[C@@H](N=[N+]=[N-])C(C)C)cc1] | ESI+: 470.3 |
| 91 | 88 | 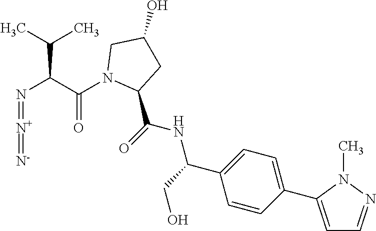 [SMILES: CC(C)[C@H](N=[N+]=[N-])C(=O)N1C[C@H](O)C[C@H]1C(=O)N[C@@H](CO)c1ccc(-c2ccnn2C)cc1] | ESI+: 456.3 |
| 92 | 88 | 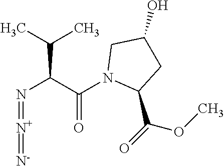 [SMILES: COC(=O)[C@@H]1C[C@@H](O)CN1C(=O)[C@@H](N=[N+]=[N-])C(C)C] | ESI+: 271.2 |
| 93 | 93 | 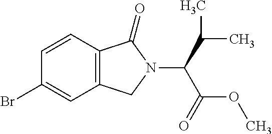 [SMILES: COC(=O)[C@H](C(C)C)N1Cc2cc(Br)ccc2C1=O] | ESI+: 328.0 |
| 94 | 94 | 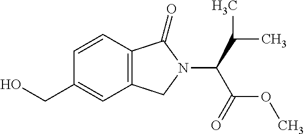 [SMILES: COC(=O)[C@H](C(C)C)N1Cc2cc(CO)ccc2C1=O] | ESI+: 278.1 |
| 95 | 95 | 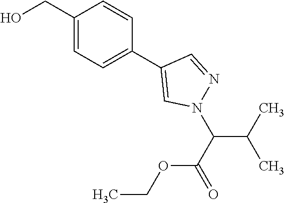 [SMILES: CCOC(=O)C(C(C)C)n1cc(-c2ccc(CO)cc2)cn1] | ESI+: 303.2 |
| 96 | 96 | 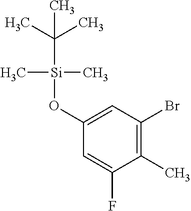 [SMILES: Cc1c(F)cc(O[Si](C)(C)C(C)(C)C)cc1Br] | ESI+: 319.1 |
| 97 | 97 | 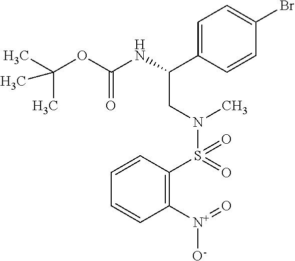 [SMILES: CN(C[C@H](NC(=O)OC(C)(C)C)c1ccc(Br)cc1)S(=O)(=O)c1ccccc1[N+](=O)[O-]] | ESI+: 536.2, 538.2 [M + Na]+ |
| 98 | 98 | 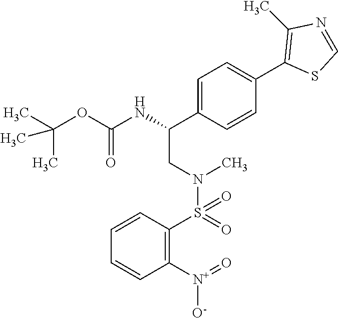 [SMILES: Cc1ncsc1-c1ccc([C@H](CN(C)S(=O)(=O)c2ccccc2[N+](=O)[O-])NC(=O)OC(C)(C)C)cc1] | ESI+: 533.2 |
| 99 | 99 | 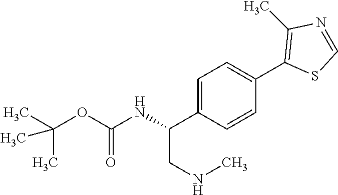 [SMILES: CNC[C@H](NC(=O)OC(C)(C)C)c1ccc(-c2scnc2C)cc1] | ESI+: 348.2 |
| 100 | 100 | 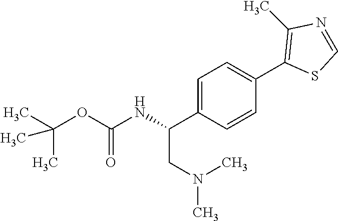 [SMILES: Cc1ncsc1-c1ccc([C@H](CN(C)C)NC(=O)OC(C)(C)C)cc1] | ESI+: 362.2 |
| 101 | 71 | 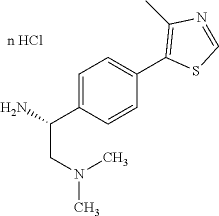 [SMILES: Cc1ncsc1-c1ccc([C@@H](N)CN(C)C)cc1] | ESI+: 262.2 |
| 102 | 102 | 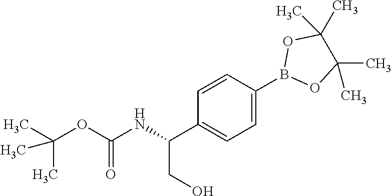 [SMILES: CC(C)(C)OC(=O)N[C@@H](CO)c1ccc(B2OC(C)(C)C(C)(C)O2)cc1] | ESI+: 386.3 [M + Na]+ |
| 103 | 103 | 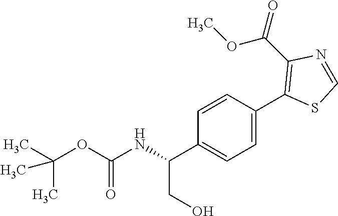 [SMILES: COC(=O)c1ncsc1-c1ccc([C@H](CO)NC(=O)OC(C)(C)C)cc1] | ESI+: 379.3 |
| 104 | 104 | 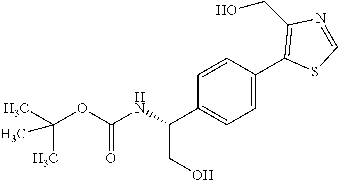 [SMILES: CC(C)(C)OC(=O)N[C@@H](CO)c1ccc(-c2scnc2CO)cc1] | ESI+: 351.2 |
| 105 | 71 | 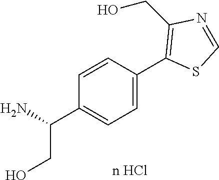 [SMILES: N[C@@H](CO)c1ccc(-c2scnc2CO)cc1] | ESI+: 251.1 |
| 106 | 106 | 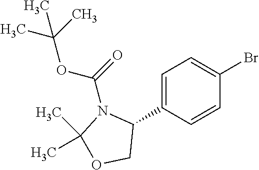 [SMILES: CC(C)(C)OC(=O)N1[C@H](c2ccc(Br)cc2)COC1(C)C] | ESI+: 378.2 [M+Na]+ |
| 107 | 107 | 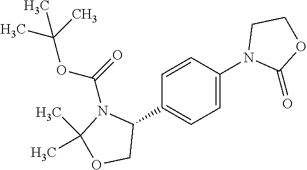 [SMILES: CC(C)(C)OC(=O)N1[C@H](c2ccc(N3CCOC3=O)cc2)COC1(C)C] | ESI+: 385.2 [M+Na]+ |
| 108 | 71 | 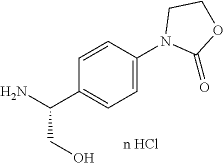 [SMILES: N[C@@H](CO)c1ccc(N2CCOC2=O)cc1] | ESI+: 245.1 [M+Na]+ |
| 109 | 109 | 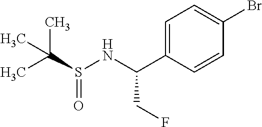 [SMILES: CC(C)(C)[S@](=O)N[C@@H](CF)c1ccc(Br)cc1] | ESI+: 323.9 |
| 110 | 98 | 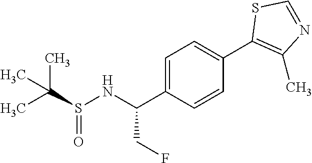 [SMILES: Cc1ncsc1-c1ccc([C@H](CF)N[S@@](=O)C(C)(C)C)cc1] | ESI+: 341.2 |
| 111 | 71 | 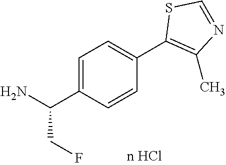 [SMILES: Cc1ncsc1-c1ccc([C@@H](N)CF)cc1] | ESI+: 237.3 |

TABLE 2

| Ex | Str |
|---|---|
| 1 | 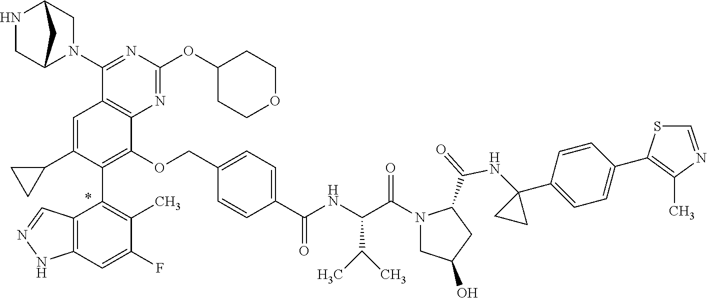 [SMILES: Cc1ncsc1-c1ccc(C2(NC(=O)[C@@H]3C[C@@H](O)CN3C(=O)[C@@H](NC(=O)c3ccc(COc4c(-c5c(C)c(F)cc6[nH]ncc56)c(C5CC5)cc5c(N6C[C@@H]7C[C@H]6CN7)nc(OC6CCOCC6)nc45)cc3)C(C)C)CC2)cc1] |
| 2 | 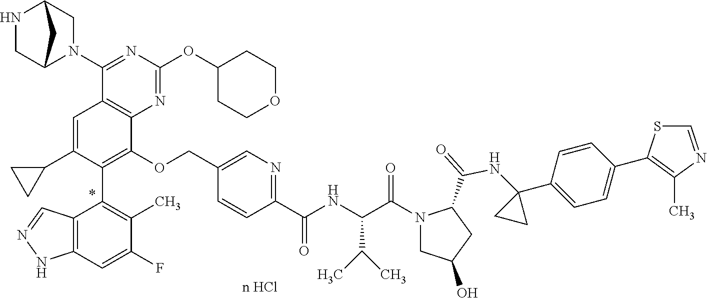 [SMILES: Cc1ncsc1-c1ccc(C2(NC(=O)[C@@H]3C[C@@H](O)CN3C(=O)[C@@H](NC(=O)c3ccc(COc4c(-c5c(C)c(F)cc6[nH]ncc56)c(C5CC5)cc5c(N6C[C@@H]7C[C@H]6CN7)nc(OC6CCOCC6)nc45)cn3)C(C)C)CC2)cc1] |
| 3 | 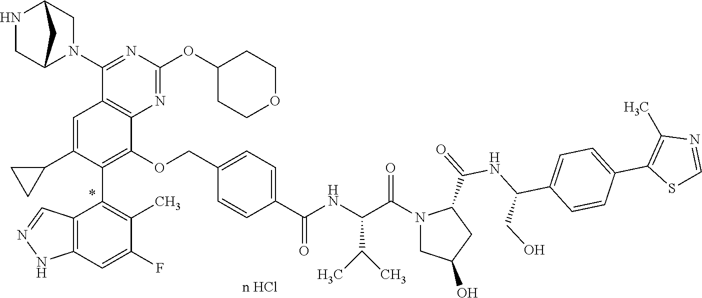 [SMILES: Cc1ncsc1-c1ccc([C@H](CO)NC(=O)[C@@H]2C[C@@H](O)CN2C(=O)[C@@H](NC(=O)c2ccc(COc3c(-c4c(C)c(F)cc5[nH]ncc45)c(C4CC4)cc4c(N5C[C@@H]6C[C@H]5CN6)nc(OC5CCOCC5)nc34)cc2)C(C)C)cc1] |
| 4 | 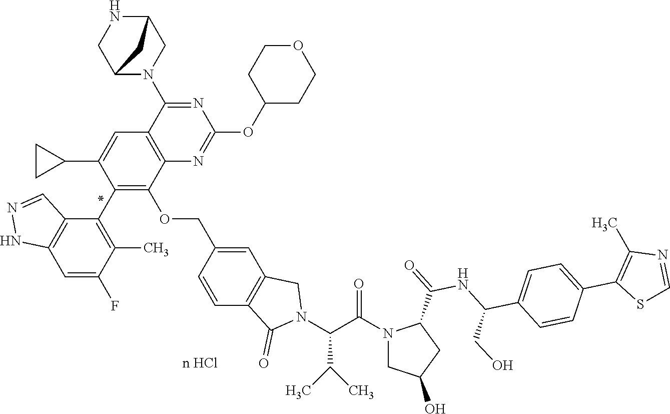 [SMILES: Cc1ncsc1-c1ccc([C@H](CO)NC(=O)[C@@H]2C[C@@H](O)CN2C(=O)[C@H](C(C)C)N2Cc3cc(COc4c(-c5c(C)c(F)cc6[nH]ncc56)c(C5CC5)cc5c(N6C[C@@H]7C[C@H]6CN7)nc(OC6CCOCC6)nc45)ccc3C2=O)cc1] |
| 5 | 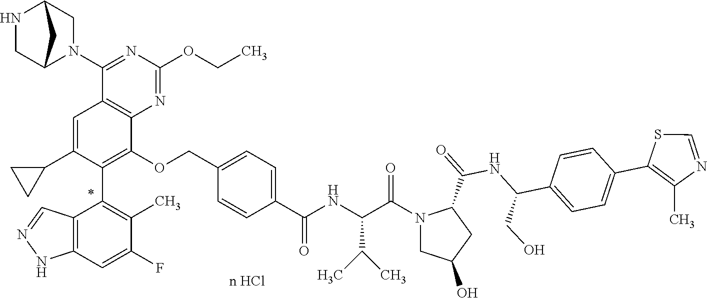 [SMILES: CCOc1nc(N2C[C@@H]3C[C@H]2CN3)c2cc(C3CC3)c(-c3c(C)c(F)cc4[nH]ncc34)c(OCc3ccc(C(=O)N[C@H](C(=O)N4C[C@H](O)C[C@H]4C(=O)N[C@@H](CO)c4ccc(-c5scnc5C)cc4)C(C)C)cc3)c2n1] |
| 6 | 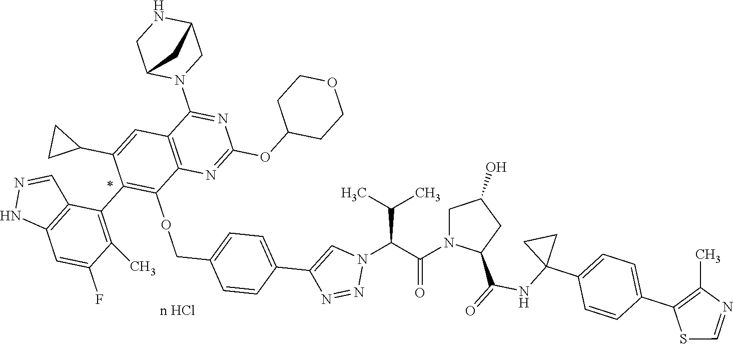 [SMILES: Cc1ncsc1-c1ccc(C2(NC(=O)[C@@H]3C[C@@H](O)CN3C(=O)[C@H](C(C)C)n3cc(-c4ccc(COc5c(-c6c(C)c(F)cc7[nH]ncc67)c(C6CC6)cc6c(N7C[C@@H]8C[C@H]7CN8)nc(OC7CCOCC7)nc56)cc4)nn3)CC2)cc1] |
| 7 | 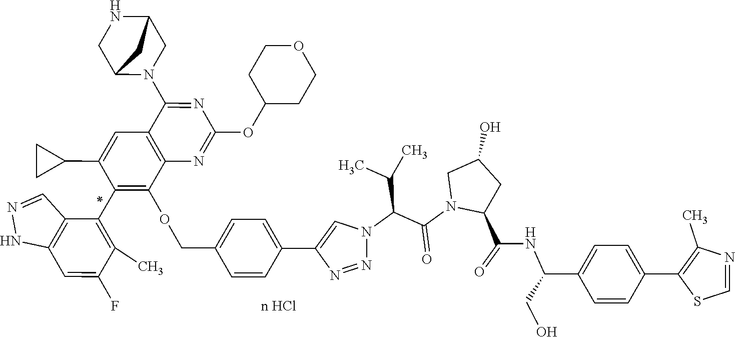 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCOCC6)nc45)cc3)nn2)cc1] |
| 8 | 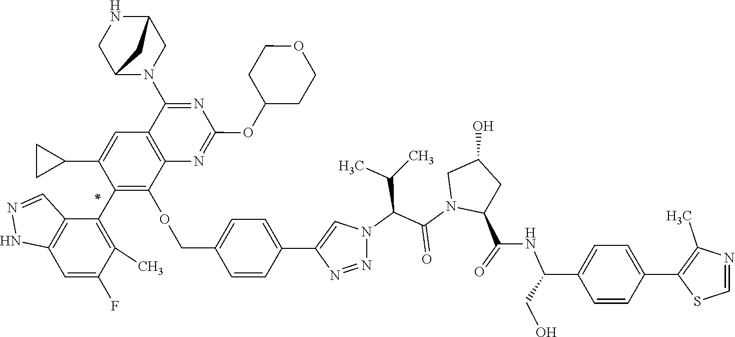 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCOCC6)nc45)cc3)nn2)cc1] |
| 9 | 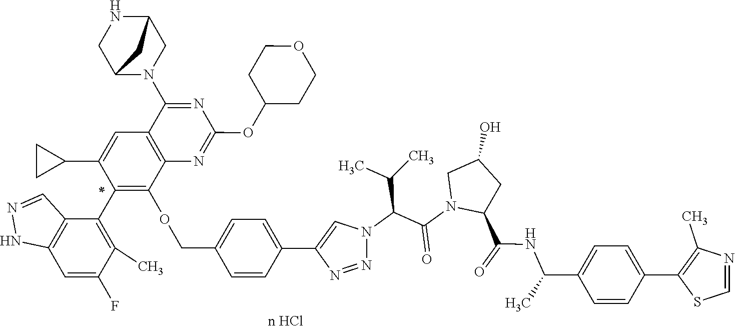 [SMILES: Cc1ncsc1-c1ccc([C@H](C)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCOCC6)nc45)cc3)nn2)cc1] |
| 10 | 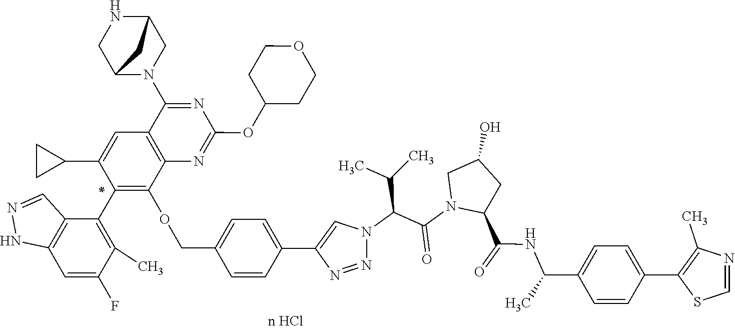 [SMILES: Cc1ncsc1-c1ccc([C@H](C)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCOCC6)nc45)cc3)nn2)cc1] |
| 11 | 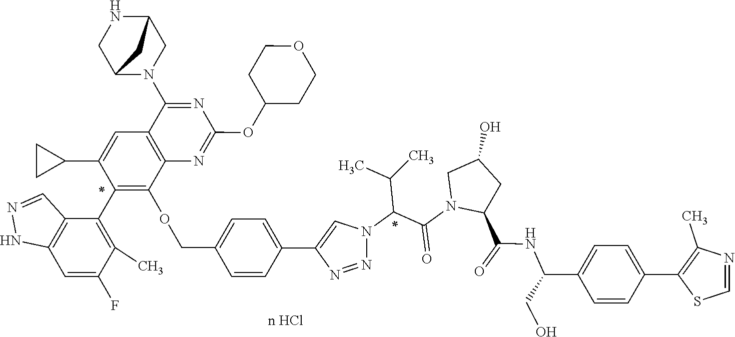 [SMILES: Cc1ncsc1-c1ccc([C@H](CO)NC(=O)[C@@H]2C[C@@H](O)CN2C(=O)C(C(C)C)n2cc(-c3ccc(COc4c(-c5c(C)c(F)cc6[nH]ncc56)c(C5CC5)cc5c(N6C[C@@H]7C[C@H]6CN7)nc(OC6CCOCC6)nc45)cc3)nn2)cc1] |
| 12 | 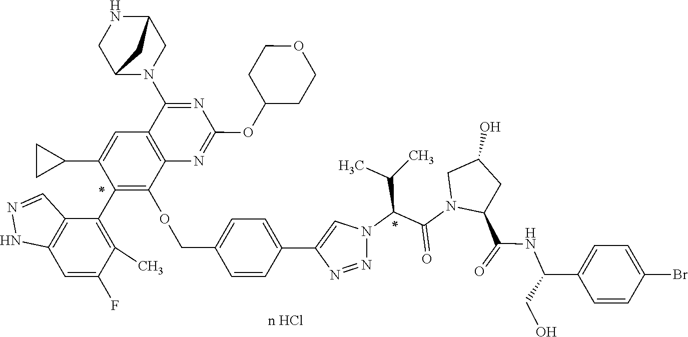 [SMILES: Cc1c(F)cc2[nH]ncc2c1-c1c(C2CC2)cc2c(N3C[C@@H]4C[C@H]3CN4)nc(OC3CCOCC3)nc2c1OCc1ccc(-c2cn([C@H](C(=O)N3C[C@H](O)C[C@H]3C(=O)N[C@@H](CO)c3ccc(Br)cc3)C(C)C)nn2)cc1] |
| 13 | 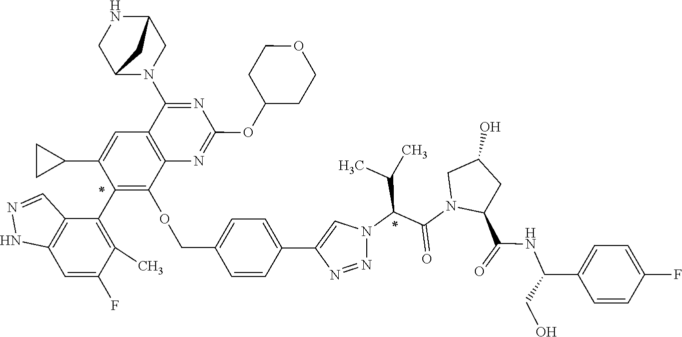 [SMILES: Cc1c(F)cc2[nH]ncc2c1-c1c(C2CC2)cc2c(N3C[C@@H]4C[C@H]3CN4)nc(OC3CCOCC3)nc2c1OCc1ccc(-c2cn([C@H](C(=O)N3C[C@H](O)C[C@H]3C(=O)N[C@@H](CO)c3ccc(F)cc3)C(C)C)nn2)cc1] |
| 14 | 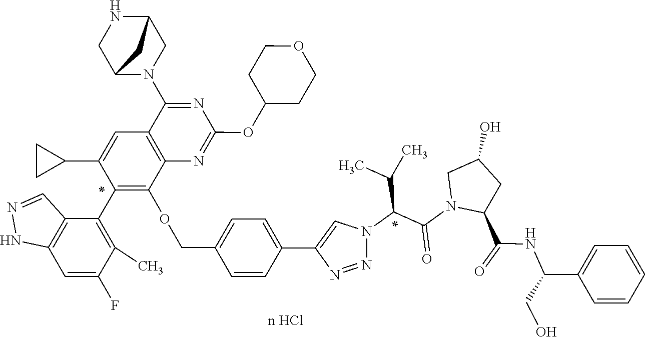 [SMILES: Cc1c(F)cc2[nH]ncc2c1-c1c(C2CC2)cc2c(N3C[C@@H]4C[C@H]3CN4)nc(OC3CCOCC3)nc2c1OCc1ccc(-c2cn([C@H](C(=O)N3C[C@H](O)C[C@H]3C(=O)N[C@@H](CO)c3ccccc3)C(C)C)nn2)cc1] |
| 15 | 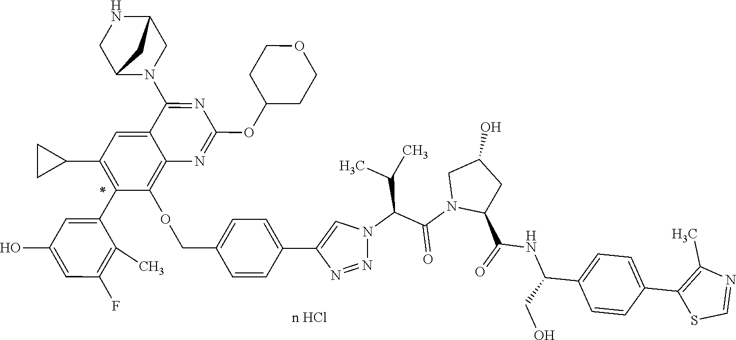 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5cc(O)cc(F)c5C)c(C5CC5)cc5c(N6C[C@@H]7C[C@H]6CN7)nc(OC6CCOCC6)nc45)cc3)nn2)cc1] |
| 16 | 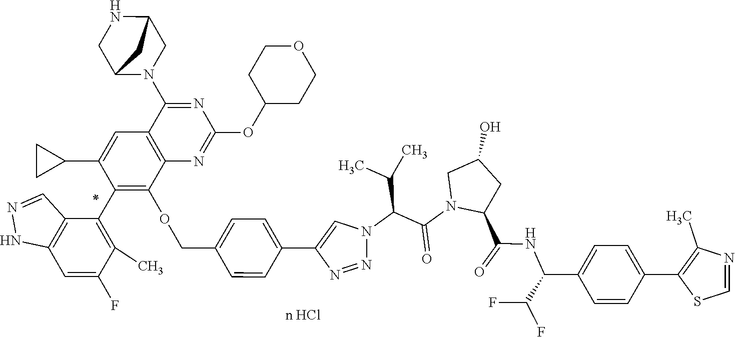 [SMILES: Cc1ncsc1-c1ccc([C@@H](NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCOCC6)nc45)cc3)nn2)C(F)F)cc1] |
| 17 | 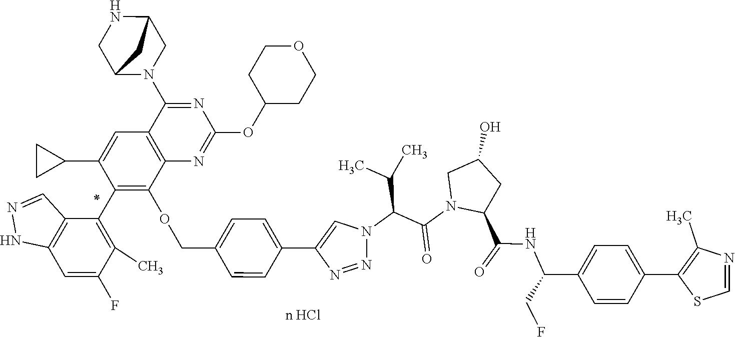 [SMILES: Cc1ncsc1-c1ccc([C@H](CF)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCOCC6)nc45)cc3)nn2)cc1] |
| 18 | 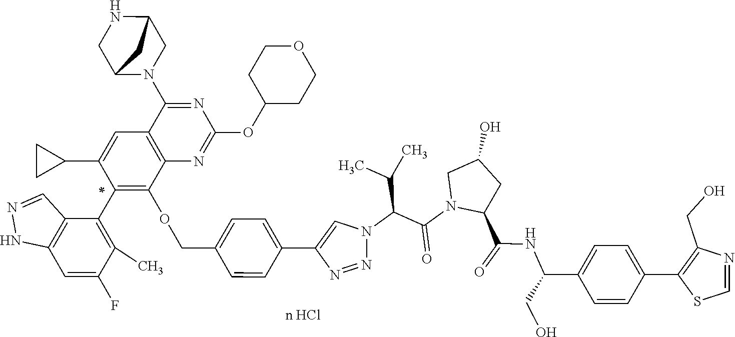 [SMILES: Cc1c(F)cc2[nH]ncc2c1-c1c(C2CC2)cc2c(N3C[C@@H]4C[C@H]3CN4)nc(OC3CCOCC3)nc2c1OCc1ccc(-c2cn([C@H](C(=O)N3C[C@H](O)C[C@H]3C(=O)N[C@@H](CO)c3ccc(-c4scnc4CO)cc3)C(C)C)nn2)cc1] |
| 19 | 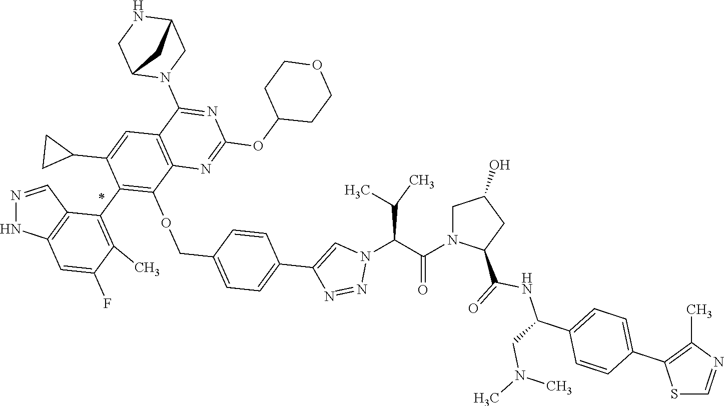 [SMILES: Cc1ncsc1-c1ccc([C@H](CN(C)C)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCOCC6)nc45)cc3)nn2)cc1] |
| 20 | 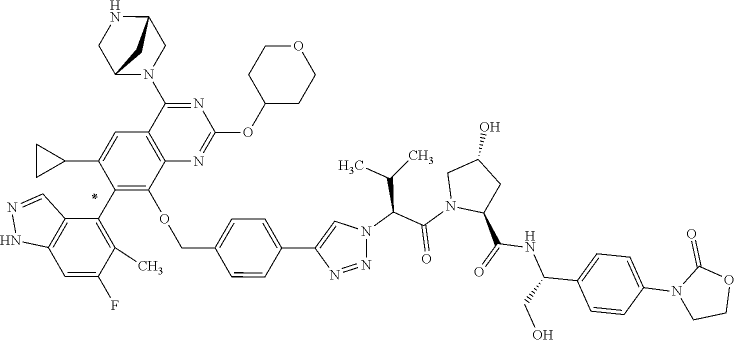 [SMILES: Cc1c(F)cc2[nH]ncc2c1-c1c(C2CC2)cc2c(N3C[C@@H]4C[C@H]3CN4)nc(OC3CCOCC3)nc2c1OCc1ccc(-c2cn([C@H](C(=O)N3C[C@H](O)C[C@H]3C(=O)N[C@@H](CO)c3ccc(N4CCOC4=O)cc3)C(C)C)nn2)cc1] |
| 21 | 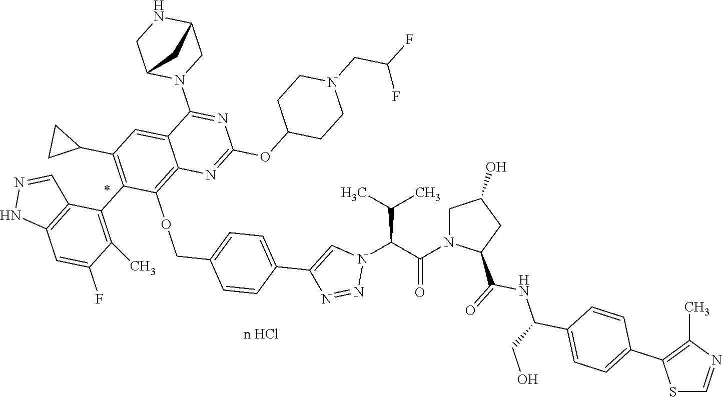 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCN(CC(F)F)CC6)nc45)cc3)nn2)cc1] |
| 22 | 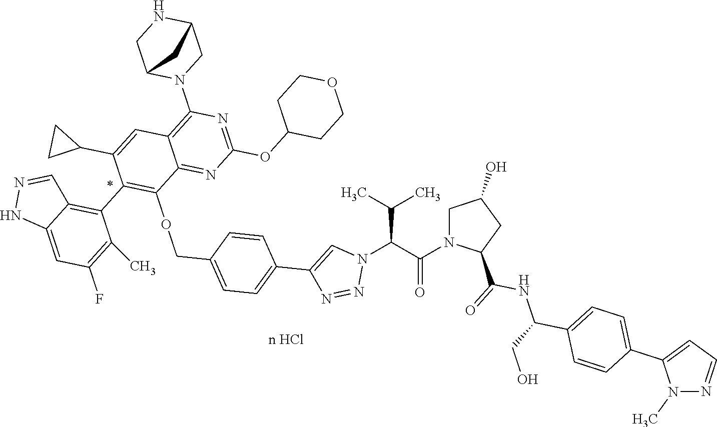 [SMILES: Cc1c(F)cc2[nH]ncc2c1-c1c(C2CC2)cc2c(N3C[C@@H]4C[C@H]3CN4)nc(OC3CCOCC3)nc2c1OCc1ccc(-c2cn([C@H](C(=O)N3C[C@H](O)C[C@H]3C(=O)N[C@@H](CO)c3ccc(-c4ccnn4C)cc3)C(C)C)nn2)cc1] |
| 23 | 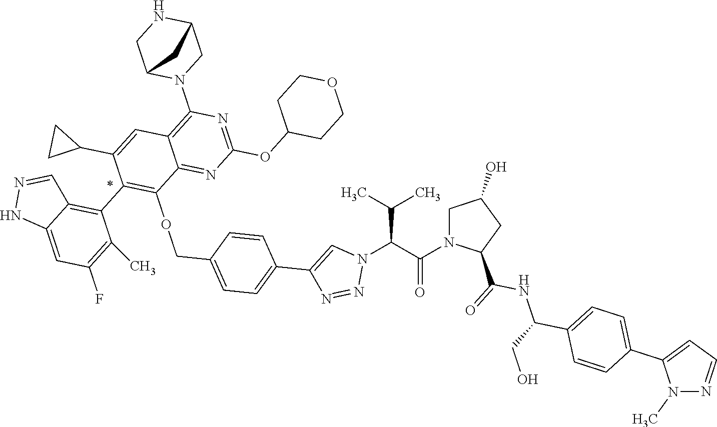 [SMILES: Cc1c(F)cc2[nH]ncc2c1-c1c(C2CC2)cc2c(N3C[C@@H]4C[C@H]3CN4)nc(OC3CCOCC3)nc2c1OCc1ccc(-c2cn([C@H](C(=O)N3C[C@H](O)C[C@H]3C(=O)N[C@@H](CO)c3ccc(-c4ccnn4C)cc3)C(C)C)nn2)cc1] |
| 24 | 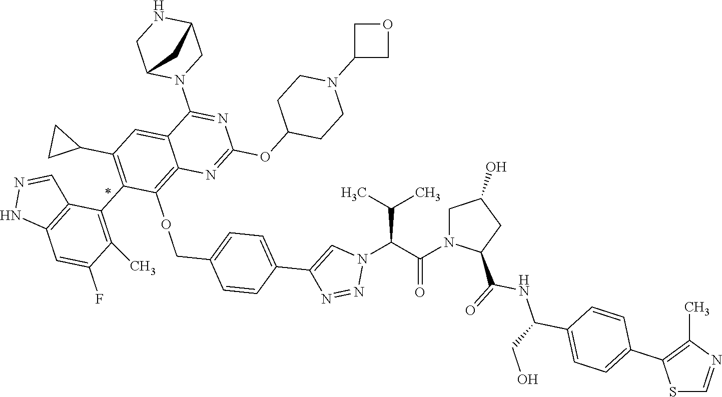 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCN(C7COC7)CC6)nc45)cc3)nn2)cc1] |
| 25 | 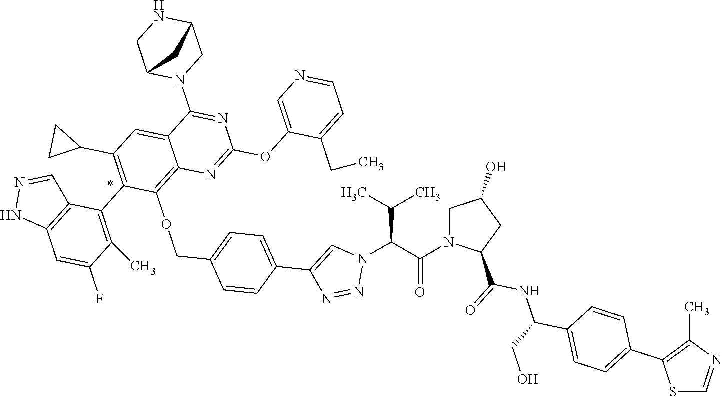 [SMILES: CCc1ccncc1Oc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@@H](CO)c5ccc(-c6scnc6C)cc5)C(C)C)nn4)cc3)c2n1] |
| 26 | 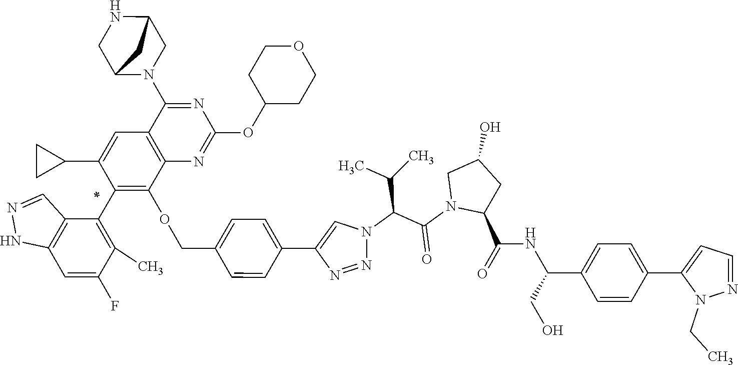 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCOCC6)nc45)cc3)nn2)cc1] |
| 27 | 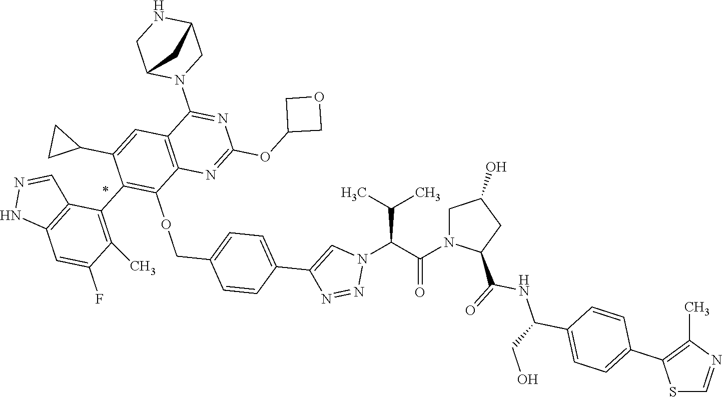 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6COC6)nc45)cc3)nn2)cc1] |
| 28 | 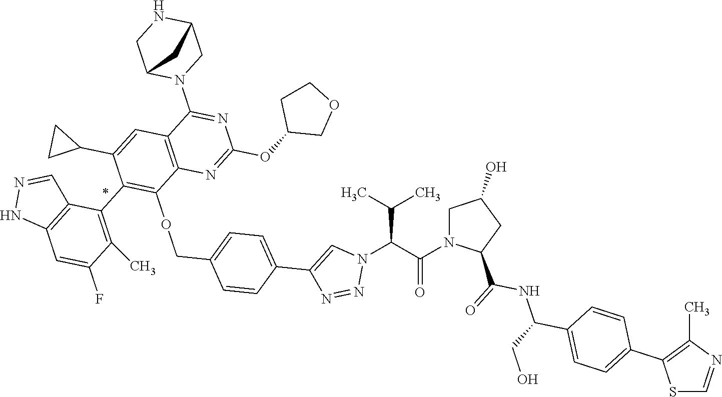 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(O[C@@H]6CCOC6)nc45)cc3)nn2)cc1] |
| 29 | 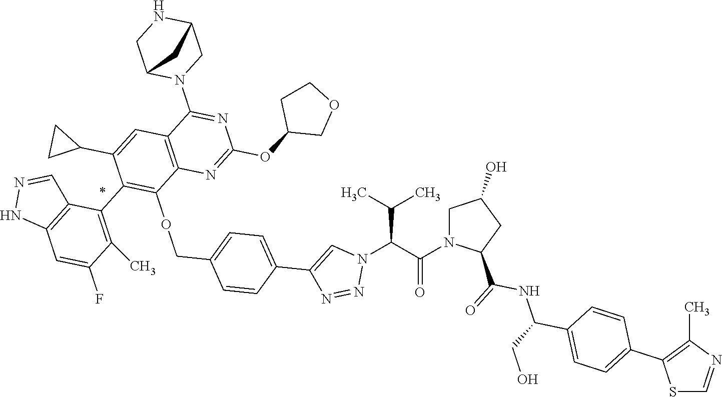 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(O[C@H]6CCOC6)nc45)cc3)nn2)cc1] |
| 48 | 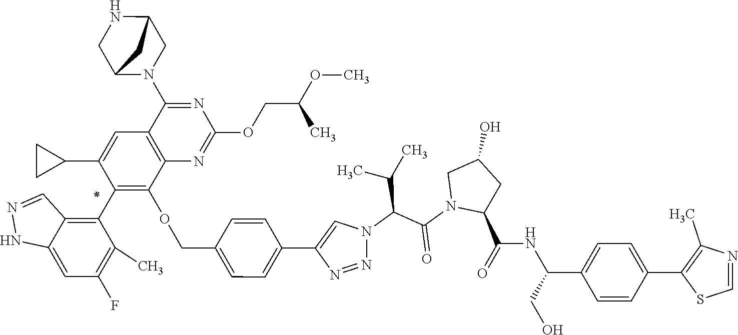 [SMILES: CO[C@@H](C)COc1nc(N2C[C@@H]3C[C@H]2CN3)c2cc(C3CC3)c(-c3c(C)c(F)cc4[nH]ncc34)c(OCc3ccc(-c4cn([C@H](C(=O)N5C[C@H](O)C[C@H]5C(=O)N[C@@H](CO)c5ccc(-c6scnc6C)cc5)C(C)C)nn4)cc3)c2n1] |
| 70 | 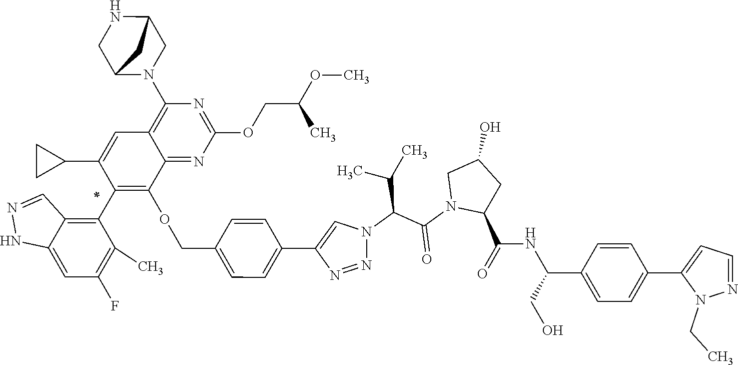 [SMILES: CCn1nccc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC[C@H](C)OC)nc45)cc3)nn2)cc1] |

TABLE 3

| Ex | Syn | DAT |
|---|---|---|
| 1 | 18# | ESI+: 1089.4 |
| 2 | 18 | ESI−: 1088.6 |
| 3 | 18 | ESI+: 1093.5 |
| 4 | 18 | ESI+: 1105.9 |
| 5 | 18 | ESI+: 1037.7 |
| 6 | 20, 7 | ESI+: 1113.3 |
| 7 | 8, 7 | ESI+: 1117.8 |
| 8 | 8 | ESI+: 1117.3 |
| | | NMR (100° C.): 0.48-0.68 (4H, m), 0.77 (3H, |
| | | br d), 1.07 (3H, br d), 1.35-1.43 (1H, |
| | | m), 1.66-1.77 (3H, m), 1.87 (1H, br d), |
| | | 1.89-1.97 (1H, m), 1.98-2.10 (3H, m), |
| | | 2.01 (3H, d), 2.10-2.21 (1H, m), 2.45 |
| | | (3H, s), 2.50-2.59 (1H, m), 3.06 (1H, |
| | | dd), 3.13 (1H, d), 3.35-3.45 (2H, m), |
| | | 3.57-3.64 (1H, m), 3.65-3.75 (3H, m), |
| | | 3.75-3.79 (1H, m), 3.80-3.90 (3H, m), |
| | | 4.16 (1H, dd), 4.35 (1H, br s), 4.41-4.48 |
| | | (1H, m), 4.48-4.56 (1H, m), 4.78-4.84 |
| | | (2H, m), 4.84-4.95 (1H, m), 5.13 (1H, br |
| | | s), 5.17-5.24 (1H, m), 5.24-5.31 (2H, m), |
| | | 6.82 (2H, d), 7.30 (1H, d), 7.38-7.44 |
| | | (4H, m), 7.44-7.48 (2H, m), 7.61 (2H, br |
| | | d), 8.00 (1H, br d), 8.43 (1H, br s), |
| | | 8.88 (1H, s), 12.75 (1H, br s) |
| 9 | 18 | ESI+: 1101.7 |
| 10 | 18 | ESI+: 1087.7 |
| 11 | 18 | ESI−: 1114.5 |
| 12 | 18 | ESI+: 1098.6, 1100.8 |
| 13 | 20 | ESI+: 1038.7 |
| 14 | 18 | ESI+: 1020.5 |
| 15 | 20, 7 | ESI+: 1093.5 |
| 16 | 18 | ESI+: 1137.8 |
| 17 | 18 | ESI+: 1119.7 |
| 18 | 18 | ESI+: 1133.3 |
| 19 | 20, 7 | ESI+: 1144.4 |
| 20 | 20 | ESI+: 1105.7 |
| 21 | 8, 7 | ESI+: 1180.3 |
| 22 | 8, 7 | ESI+: 1100.4 |
| 23 | 8 | ESI+: 1100.4 |
| 24 | 8 | ESI+: 1173.2 |
| 25 | 18# | ESI+: 1160.6 [M + Na]+ |
| 26 | 8 | ESI+: 1114.5 |
| | | NMR (100° C.): 0.48-0.68 (4H, m), 0.77 (3H, |
| | | br d), 1.03-1.10 (3H, m), 1.30 (3H, t), |
| | | 1.35-1.43 (1H, m), 1.66-1.77 (3H, m), |
| | | 1.87 (1H, br d), 1.89-1.97 (1H, m), 1.98- |
| | | 2.10 (3H, m), 2.01 (3H, d), 2.11-2.20 |
| | | (1H, m), 2.50-2.60 (1H, m), 3.06 (1H, |
| | | dd), 3.13 (1H, d), 3.35-3.45 (2H, m), |
| | | 3.57-3.64 (1H, m), 3.65-3.75 (3H, m), |
| | | 3.75-3.79 (1H, m), 3.80-3.93 (3H, m), |
| | | 4.11 (2H, q), 4.16 (1H, dd), 4.35 (1H, br |
| | | s), 4.41-4.48 (1H, m), 4.49-4.57 (1H, m), |
| | | 4.78-4.84 (2H, m), 4.87-4.96 (1H, m), |
| | | 5.12 (1H, br s), 5.17-5.24 (1H, m), 5.24- |
| | | 5.32 (2H, m), 6.27 (1H, d), 6.82 (2H, d), |
| | | 7.30 (1H, d), 7.35-7.40 (2H, m), 7.40- |
| | | 7.48 (5H, m), 7.61 (2H, br d), 8.01 (1H, |
| | | br d), 8.43 (1H, s), 12.75 (1H, br s) |
| 27 | 8 | ESI+: 1089.5 |
| 28 | 8 | ESI+: 1103.3 |
| 29 | 8 | ESI+: 1103.4 |
| 48 | 48 | ESI+: 1105.7 |
| | | NMR (100° C.): 0.48-0.68 (4H, m), 0.77 (3H, |
| | | br d), 1.04-1.10 (3H, m), 1.14 (3H, d), |
| | | 1.35-1.43 (1H, m), 1.74 (1H, br d), 1.87 |
| | | (1H, br d), 1.89-1.97 (1H, m), 2.00 (3H, |
| | | d), 2.01-2.10 (1H, m), 2.10-2.32 (1H, m), |
| | | 2.45 (3H, s), 2.45-2.60 (1H, m), 3.06 |
| | | (1H, dd), 3.14 (1H, d), 3.30 (3H, s), |
| | | 3.60 (1H, br d), 3.65-3.78 (5H, m), 3.84 |
| | | (1H, dd), 4.16 (1H, dd), 4.28 (1H, dd), |
| | | 4.32-4.38 (2H, m), 4.45 (1H, br t), 4.52 |
| | | (1H, br t), 4.78-4.85 (2H, m), 4.86-4.94 |
| | | (1H, m), 5.14 (1H, br s), 5.28 (2H, d), |
| | | 6.83 (2H, d), 7.30 (1H, d), 7.37-7.43 |
| | | (4H, m), 7.45 (1H, d), 7.47 (1H, s), 7.59 |
| | | (2H, br d), 8.00 (1H, br d), 8.42 (1H, |
| | | s), 8.88 (1H, s), 12.75 (1H, br s) |
| 70 | 70 | ESI+: 1102.8 |
| | | NMR (100° C.): 0.48-0.68 (4H, m), 0.77 (3H, |
| | | br d), 1.07 (3H, d), 1.14 (3H, d), 1.30 |
| | | (3H, t), 1.35-1.43 (1H, m), 1.75 (1H, br |
| | | d), 1.85-1.98 (2H, m), 2.00 (3H, d), |
| | | 2.01-2.10 (1H, m), 2.42-2.51 (1H, m), |
| | | 2.51-2.60 (1H, m), 3.07 (1H, dd), 3.16 |
| | | (1H, d), 3.30 (3H, s), 3.60 (1H, br d), |
| | | 3.65-3.78 (4H, m), 3.80 (1H, br s), 3.84 |
| | | (1H, br dd), 4.07-4.15 (2H, m), 4.17 (1H, |
| | | dd), 4.28 (1H, dd), 4.32-4.39 (2H, m), |
| | | 4.42-4.48 (1H, m), 4.52 (1H, br t), 4.73- |
| | | 4.85 (2H, m), 4.87-4.96 (1H, m), 5.14 |
| | | (1H, br s), 5.28 (2H, br d), 6.27 (1H, |
| | | d), 6.83 (2H, d), 7.30 (1H, d), 7.35-7.40 |
| | | (2H, m), 7.40-7.49 (5H, m), 7.59 (2H, br |
| | | d), 8.01 (1H, br d), 8.42 (1H, s), 12.75 |
| | | (1H, br s) |

### Test Example A: Evaluation of Anti-Tumor Activity in Human KRAS G12D Mutation Positive GP2d Colorectal Cancer Cell Line-Derived Xenograft Mice

GP2d cells (ECACC, 95090714) were cultured using DMEM medium (Dulbecco's modified eagle medium) having 10% fetal bovine serum and 2 mM L-glutamine added therein in the presence of 5% CO₂ at 37° C. The GP2d cells were collected and suspended in PBS, an equal amount of Matrigel (from Corning Incorporated) was added thereto to prepare a cell suspension of 3.0×10⁶ Cells/200 μL. 6-8-Week old female nude mice (BALB/c nude mice, from Beijing Vital River Laboratory Animal Technology Co., Ltd) were subcutaneously inoculated with the cell suspension in a volume of 200 μL per mouse. After one week of the inoculation, the mice were divided into groups so that all the groups had approximately the same tumor volume, administration of a test compound was started. The study was conducted for 5 mice each of a vehicle group and a test compound administration group. To the vehicle group, a solvent was administered in the tail vein, and to the test compound administration group, the test compound dissolved in the solvent was administered in the tail vein. The solvent was prepared by mixing 4% by volume of ethanol, 1% by volume of 50% (2-hydroxypropyl)-β-cyclodextrin, 9% by volume of polyoxyethylene hydrogenated castor oil (HCO-40), and 86% by volume of 5% glucose solution. The administration was performed once a week and three times in total. The tumor size and the body weight were measured twice to three times a week. The tumor volume was calculated using the following formula.

$$[ Tumor volume ( {mm}^{{}_{3}} ) ] = [ long diameter of the tumor ( mm ) ] \times {[ short diameter of the tumor ( mm ) ]}^{2} \times 0.5$$

The tumor growth inhibition rate (%) by the test compound was calculated as 100% inhibition of the tumor volume of the test compound-treated group on the start day of treatment and 0% inhibition of the tumor volume of the vehicle group at the end of 3 weeks after the first treatment.

TABLE 4

| | Amount of | Anti-tumor activity |
|---|---|---|
| | administration | after 3 weeks of |
| Ex | (mg/kg) | initial administration |
| 8 | 30 | 85% inhibition |
| 26 | 30 | 90% inhibition |
| 48 | 30 | 85% inhibition |
| 70 | 30 | 74% inhibition |

### Test Example B: Evaluation of Anti-Tumor Activity in KRAS G12D Mutation Positive Human Lung Cancer Patient-Derived Xenograft Mice

4-6 Week-old female nude mice (Crl: NMRI-Foxninu, from Charles River) were warehoused and were subcutaneously inoculated with human lung cancer patient-derived tumor (Model name: LXFA 2204 (from Charles River)). The mice were divided into groups after about one month of the inoculation, and administration of a test compound was started. The study was conducted for 8 mice each of a vehicle group and a test compound administration group. To the vehicle group, a solvent was administered in the tail vein, and to the test compound administration group, the test compound dissolved in the solvent was administered in the tail vein. The solvent was prepared by mixing 4% by volume of ethanol, 1% by volume of 50% (2-hydroxypropyl)-β-cyclodextrin, 9% by volume of polyoxyethylene hydrogenated castor oil (HCO-40), and 86% by volume of 5% glucose solution. The administration to the test compound group was performed twice a week and 6 times in total. The tumor size and the body weight were measured twice a week. The tumor volume was calculated using the following formula.

$$[ Tumor volume ( {mm}^{{}_{3}} ) ] = [ long diameter of the tumor ( mm ) ] \times {[ short diameter of the tumor ( mm ) ]}^{2} \times 0.5$$

The tumor growth inhibition rate (%) by the test compound was calculated as 100% inhibition of the tumor volume of the test compound-treated group on the day before the start of treatment and 0% inhibition of the tumor volume of the vehicle group at the end of 3 weeks after the first treatment. If the tumor volume of the test compound-treated group was less than the tumor volume of the day before the start of treatment, the tumor regression rate (%) of the test compound was calculated, assuming that the tumor volume of the day before the start of treatment was 0% regression and the tumor volume of 0 was 100% regression.

TABLE 5

| | Amount of | Anti-tumor activity |
|---|---|---|
| | administration | after 3 weeks of |
| Ex | (mg/kg) | initial administration |
| 8 | 30 | 18% regression |
| 26 | 30 | 79% inhibition |
| 48 | 30 | 79% inhibition |
| 70 | 30 | 77% inhibition |

### INDUSTRIAL APPLICABILITY

The compound or a salt thereof of the present invention is excellent in the degradation-inducing action on a G12D mutant KRAS protein, is useful as a G12D mutant KRAS inhibitor, and can be used as an active ingredient of a pharmaceutical composition, for example, a pharmaceutical composition for treating a cancer of colorectal cancer and/or Lung cancer.

1. A pharmaceutical composition comprising a compound of the following formula (I) or a salt thereof and one or more pharmaceutically acceptable excipients: 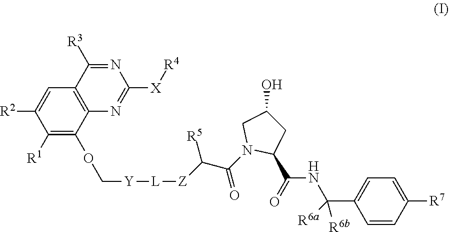
wherein
R¹ is the following formula (II) or (III): 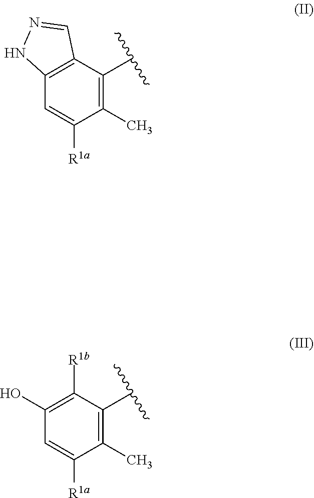
R^{1a} and R^{1b}, which are the same as or different from each other, are H or F,
R² is halogen, C₁₋₃ alkyl, cyclopropyl, or vinyl,
R³ is the following formula (IV): 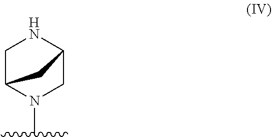 [SMILES: *N1C[C@@H]2C[C@H]1CN2]
R⁴ is C₁₋₃ alkyl, oxetanyl, tetrahydrofuranyl, tetrahydropyranyl, optionally substituted pyrazolyl, optionally substituted pyridyl, optionally substituted pyrimidinyl, optionally substituted pyrrolidinyl, or optionally substituted piperidinyl,
R⁵ is ethyl, isopropyl, tert-butyl, or C₃₋₆ cycloalkyl,
R^{6a} and R^{6b}, which are the same as or different from each other, are H or C₁₋₃ alkyl optionally substituted with a group selected from the group consisting of F, OH, and N(CH₃)₂, or R^{6a} and R^{6b} form cyclopropyl together with the carbon to which they are attached,
R⁷ is H, halogen, or a group selected from the group consisting of the following formulae (VI), (VII), (VIII), and (IX): 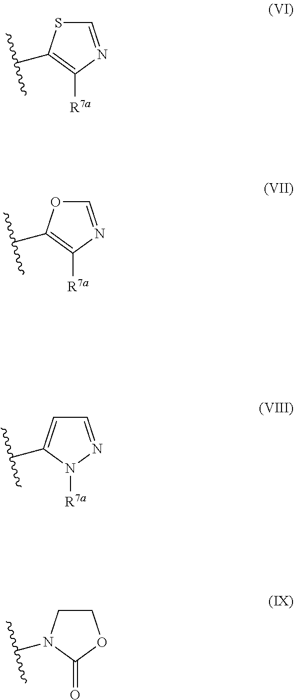
R^{7a} is H or C₁₋₃ alkyl optionally substituted with OH,
X is O,
Y is phenylene or pyridinediyl,
L is a bond, C₁₋₃ alkylene, or C═O,
Z is NH or a group selected from the group consisting of the following formulae (X), (XI), and (XII): 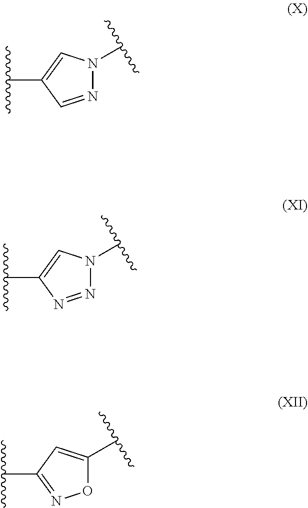 [SMILES: *c1cnn(*)c1 ; *c1cn(*)nn1 ; *c1cc(*)on1]
or, Y-L-Z is the following formula (XIII):

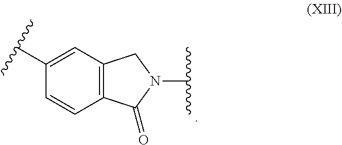 [SMILES: *c1ccc2c(c1)CN(*)C2=O]

2. The pharmaceutical composition according to claim 1, wherein the compound of the formula (I) is selected from the group consisting of
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide,
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-[(1R)-2-hydroxy-1-{4-[4-(hydroxymethyl)-1,3-thiazol-5-yl]phenyl}ethyl]-L-prolinamide,
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(2-oxo-1,3-oxazolidin-3-yl)phenyl]ethyl}-L-prolinamide,
(4R)-1-[(2S)-2-(4-{4-[({6-cyclopropyl-4-[(1S,4S)-2,5-diazabicyclo[2.2.1]heptan-2-yl]-2-{[1-(2,2-difluoroethyl) piperidin-4-yl]oxy}-7-(6-fluoro-5-methyl-1H-indazol-4-yl)quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide, (4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(1-methyl-1H-pyrazol-5-yl)phenyl]ethyl}-L-prolinamide, and
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-N-{(1R)-1-[4-(1-ethyl-1H-pyrazol-5-yl)phenyl]-2-hydroxyethyl}-4-hydroxy-L-prolinamide.

3-5. (canceled)

6. A method of treating at least one of colorectal cancer and lung cancer, the method comprising administering an effective amount of a compound of the following formula (I) or a salt thereof to a subject: 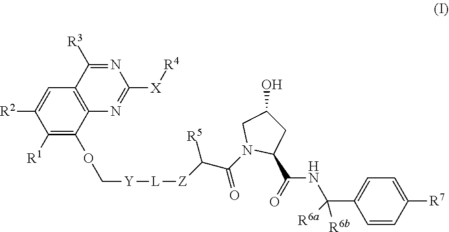
wherein
R¹ is the following formula (II) or (III): 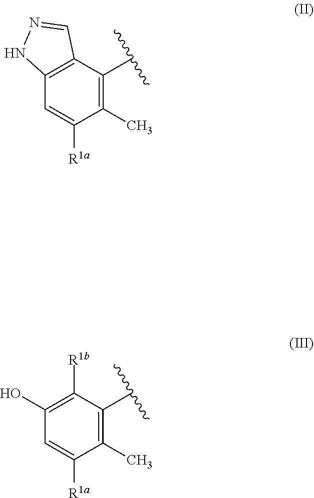
R^{1a} and R^{1b}, which are the same as or different from each other, are H or F,
R² is halogen, C₁₋₃ alkyl, cyclopropyl, or vinyl,
R³ is the following formula (IV): 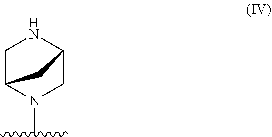 [SMILES: *N1C[C@@H]2C[C@H]1CN2]
R⁴ is C₁₋₃ alkyl, oxetanyl, tetrahydrofuranyl, tetrahydropyranyl, optionally substituted pyrazolyl, optionally substituted pyridyl, optionally substituted pyrimidinyl, optionally substituted pyrrolidinyl, or optionally substituted piperidinyl,
R⁵ is ethyl, isopropyl, tert-butyl, or C₃₋₆ cycloalkyl,
R^{6a} and R^{6b}, which are the same as or different from each other, are H or C₁₋₃ alkyl optionally substituted with a group selected from the group consisting of F, OH, and N(CH₃)₂, or R^{6a} and R^{6b} form cyclopropyl together with the carbon to which they are attached,
R⁷ is H, halogen, or a group selected from the group consisting of the following formulae (VI), (VII), (VIII), and (IX): 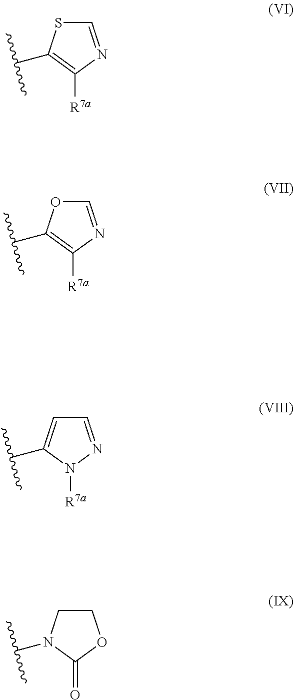
R^{7a} is H or C₁₋₃ alkyl optionally substituted with OH,
X is O,
Y is phenylene or pyridinediyl,
L is a bond, C₁₋₃ alkylene, or C═O,
Z is NH or a group selected from the group consisting of the following formulae (X), (XI), and (XII): 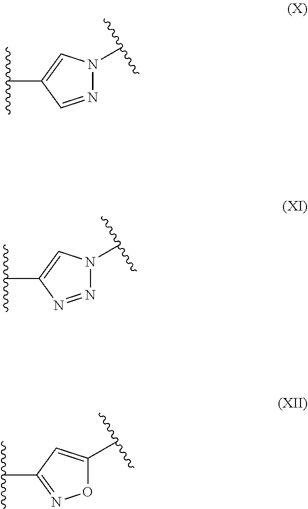 [SMILES: *c1cnn(*)c1 ; *c1cn(*)nn1 ; *c1cc(*)on1]
or, Y-L-Z is the following formula (XIII):

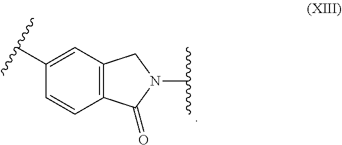 [SMILES: *c1ccc2c(c1)CN(*)C2=O]

7. A pharmaceutical composition comprising a compound or a salt thereof and one or more pharmaceutically acceptable excipients,
wherein the compound is selected from the group consisting of
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide,
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-[(1R)-2-hydroxy-1-{4-[4-(hydroxymethyl)-1,3-thiazol-5-yl]phenyl}ethyl]-L-prolinamide,
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(2-oxo-1,3-oxazolidin-3-yl)phenyl]ethyl}-L-prolinamide,
(4R)-1-[(2S)-2-(4-{4-[({6-cyclopropyl-4-[(1S,4S)-2,5-diazabicyclo[2.2.1]heptan-2-yl]-2-{[1-(2,2-difluoroethyl) piperidin-4-yl]oxy}-7-(6-fluoro-5-methyl-1H-indazol-4-yl)quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide,
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(1-methyl-1H-pyrazol-5-yl)phenyl]ethyl}-L-prolinamide,
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-N-{(1R)-1-[4-(1-ethyl-1H-pyrazol-5-yl)phenyl]-2-hydroxyethyl}-4-hydroxy-L-prolinamide,
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(2S)-2-methoxypropoxy]quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide, and
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(2S)-2-methoxypropoxy]quinazolin-8-yl}oxy)methyl]phenyl}-1H-1,2,3-triazol-1-yl)-3-methylbutanoyl]-N-{(1R)-1-[4-(1-ethyl-1H-pyrazol-5-yl)phenyl]-2-hydroxyethyl}-4-hydroxy-L-prolinamide.

8. The pharmaceutical composition according to claim 7, wherein the compound is selected from the group consisting of
(4R)-1-[(2S)-2-(4-{4-[({(7M)-6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide,
(4R)-1-[(2S)-2-(4-{4-[({(7M)-6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-[(1R)-2-hydroxy-1-{4-[4-(hydroxymethyl)-1,3-thiazol-5-yl]phenyl}ethyl]-L-prolinamide,
(4R)-1-[(2S)-2-(4-{4-[({(7M)-6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(2-oxo-1,3-oxazolidin-3-yl)phenyl]ethyl}-L-prolinamide,
(4R)-1-[(2S)-2-(4-{4-[({(7M)-6-cyclopropyl-4-[(1S,4S)-2,5-diazabicyclo[2.2.1]heptan-2-yl]-2-{[1-(2,2-difluoroethyl) piperidin-4-yl]oxy}-7-(6-fluoro-5-methyl-1H-indazol-4-yl)quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide,
(4R)-1-[(2S)-2-(4-{4-[({(7M)-6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(1-methyl-1H-pyrazol-5-yl)phenyl]ethyl}-L-prolinamide,
(4R)-1-[(2S)-2-(4-{4-[({(7M)-6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-N-{(1R)-1-[4-(1-ethyl-1H-pyrazol-5-yl)phenyl]-2-hydroxyethyl}-4-hydroxy-L-prolinamide,
(4R)-1-[(2S)-2-(4-{4-[({(7M)-6-cyclopropyl-4-[(1S,4S)-2,5-diazabicyclo[2.2.1]heptan-2-yl]-7-(6-fluoro-5-methyl-1H-indazol-4-yl)-2-[(2S)-2-methoxypropoxy]quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide, and
(4R)-1-[(2S)-2-(4-{4-[({(7M)-6-cyclopropyl-4-[(1S,4S)-2,5-diazabicyclo[2.2.1]heptan-2-yl]-7-(6-fluoro-5-methyl-1H-indazol-4-yl)-2-[(2S)-2-methoxypropoxy]quinazolin-8-yl}oxy)methyl]phenyl}-1H-1,2,3-triazol-1-yl)-3-methylbutanoyl]-N-{(1R)-1-[4-(1-ethyl-1H-pyrazol-5-yl)phenyl]-2-hydroxyethyl}-4-hydroxy-L-prolinamide.

9. The pharmaceutical composition according to claim 7, wherein the compound is selected from the group consisting of
(4R)-1-[(2S)-2-(4-{4-[({(7P)-6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide,
(4R)-1-[(2S)-2-(4-{4-[({(7P)-6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-[(1R)-2-hydroxy-1-{4-[4-(hydroxymethyl)-1,3-thiazol-5-yl]phenyl}ethyl]-L-prolinamide,
(4R)-1-[(2S)-2-(4-{4-[({(7P)-6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(2-oxo-1,3-oxazolidin-3-yl)phenyl]ethyl}-L-prolinamide,
(4R)-1-[(2S)-2-(4-{4-[({(7P)-6-cyclopropyl-4-[(1S,4S)-2,5-diazabicyclo[2.2.1]heptan-2-yl]-2-{[1-(2,2-difluoroethyl) piperidin-4-yl]oxy}-7-(6-fluoro-5-methyl-1H-indazol-4-yl)quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide,
(4R)-1-[(2S)-2-(4-{4-[({(7P)-6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(1-methyl-1H-pyrazol-5-yl)phenyl]ethyl}-L-prolinamide,
(4R)-1-[(2S)-2-(4-{4-[({(7P)-6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-N-{(1R)-1-[4-(1-ethyl-1H-pyrazol-5-yl)phenyl]-2-hydroxyethyl}-4-hydroxy-L-prolinamide,
(4R)-1-[(2S)-2-(4-{4-[({(7P)-6-cyclopropyl-4-[(1S,4S)-2,5-diazabicyclo[2.2.1]heptan-2-yl]-7-(6-fluoro-5-methyl-1H-indazol-4-yl)-2-[(2S)-2-methoxypropoxy]quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide, and
(4R)-1-[(2S)-2-(4-{4-[({(7P)-6-cyclopropyl-4-[(1S,4S)-2,5-diazabicyclo[2.2.1]heptan-2-yl]-7-(6-fluoro-5-methyl-1H-indazol-4-yl)-2-[(2S)-2-methoxypropoxy]quinazolin-8-yl}oxy)methyl]phenyl}-1H-1,2,3-triazol-1-yl)-3-methylbutanoyl]-N-{(1R)-1-[4-(1-ethyl-1H-pyrazol-5-yl)phenyl]-2-hydroxyethyl}-4-hydroxy-L-prolinamide.

10-12. (canceled)

13. A method of treating at least one of colorectal cancer and lung cancer, the method comprising administering an effective amount of a compound or a salt thereof to a subject,
wherein the compound is selected from the group consisting of
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide,
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-[(1R)-2-hydroxy-1-{4-[4-(hydroxymethyl)-1,3-thiazol-5-yl]phenyl}ethyl]-L-prolinamide,
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(2-oxo-1,3-oxazolidin-3-yl)phenyl]ethyl}-L-prolinamide,
(4R)-1-[(2S)-2-(4-{4-[({6-cyclopropyl-4-[(1S,4S)-2,5-diazabicyclo[2.2.1]heptan-2-yl]-2-{[1-(2,2-difluoroethyl) piperidin-4-yl]oxy}-7-(6-fluoro-5-methyl-1H-indazol-4-yl)quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide,
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-4-hydroxy-N-{(1R)-2-hydroxy-1-[4-(1-methyl-1H-pyrazol-5-yl)phenyl]ethyl}-L-prolinamide,
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(oxan-4-yl)oxy]quinazolin-8-yl}oxy)methyl]phenyl}-1H-1,2,3-triazol-1-yl)-3-methylbutanoyl]-N-{(1R)-1-[4-(1-ethyl-1H-pyrazol-5-yl)phenyl]-2-hydroxyethyl}-4-hydroxy-L-prolinamide,
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(2S)-2-methoxypropoxy]quinazolin-8-yl}oxy)methyl]phenyl}-1H-1,2,3-triazol-1-yl)-3-methylbutanoyl]-4-hydroxy-N-{(1R)-2-hydroxy-1-[4-(4-methyl-1,3-thiazol-5-yl)phenyl]ethyl}-L-prolinamide, and
(4R)-1-[(2S)-2-(4-{4-[({6-cyclopropyl-4-[(1S,4S)-2,5-diazabicyclo[2.2.1]heptan-2-yl]-7-(6-fluoro-5-methyl-1H-indazol-4-yl)-2-[(2S)-2-methoxypropoxy]quinazolin-8-yl}oxy)methyl]phenyl}-1H-1,2,3-triazol-1-yl)-3-methylbutanoyl]-N-{(1R)-1-[4-(1-ethyl-1H-pyrazol-5-yl)phenyl]-2-hydroxyethyl}-4-hydroxy-L-prolinamide.
